SEQUENCE LISTING (56 sequences)

SEQ ID NO: 1 - protein, 288 aa, synthetic construct
  REGION 1..288: note = Neisseria gonorrhoeae MetQ protein
MKTFFKTLSAAALALILAACGGQKDSAPAASAAAPSADNGAAKKEIVFGTTVGDFGDMVKEQIQAELEKKGYTVKLVEFTDYVRPNLALAEGELDINVFQHKPYLDDFKKEHNLDITEAFQVPTAPLGLYPGKLKSLEEVKDGSTVSAPNDPSNFARALVMLNELGWIKLKDGINPLTASKADIAENLKNIKIVELEAAQLPRSRADVDFAVVNGNYAISSGMKLTEALFQEPSFAYVNWSAVKTADKDSQWLKDVTEAYNSDAFKAYAHKRFEGYKYPAAWNEGAAK

SEQ ID NO: 2 - DNA, 880 bp, synthetic construct
  misc_feature 1..880: note = Neisseria gonorrhoeae MetQ DNA Sequence
  source 1..880: mol_type = other DNA
atgaaaaccttcttcaaaaccctttccgccgccgcactcgcgctcatcctcgcagcctgcggcggtcaaaaagacagcgcgcccgcagcctctgccgccgccccttctgccgataacggcgcggcgaaaaaagaaatcgtcttcggcacgaccgtcggcgacttcggcgatatggtcaaagaacaaatccaagccgagctggagaaaaaaggctacaccgtcaaattggtcgaatttaccgactatgtgcgcccgaatctggcattggcggagggcgagttggacatcaacgtcttccaacacaaaccctatcttgacgatttcaaaaaagaacacaacctggacatcaccgaagccttccaagtgccgaccgcgcctttgggactgtatccgggcaaactgaaatcgctggaagaagtcaaagacggcagcaccgtatccgcgcccaacgacccgtccaacttcgcacgcgccttggtgatgctgaacgaactgggttggatcaaactcaaagacggcatcaatccgctgaccgcatccaaagccgacatcgcggaaaacctgaaaaacatcaaaatcgtcgagcttgaagccgcacaactgccgcgcagccgcgccgacgtggattttgccgtcgtcaacggcaactacgccataagcagcggcatgaagctgaccgaagccctgttccaagagccgagctttgcctatgtcgcctggtctgccgtcaaaaccgccgacaaagacagccaatggcttaaagacgtaaccgaggcctataactccgacgcgttcaaagcctacgcgcacaaacgcttcgagggctacaaataccctgccgcatggaatgaaggcgcagccaaataaggcagtcgtataa

SEQ ID NO: 3 - protein, 288 aa, synthetic construct
  REGION 1..288: note = Neisseria gonorrhoeae MetQSM protein
MKTFFKTLSAAALALILAACGGQKDSAPAASAAAPSADNGAAKKEIVFGTTVGDFGDMVKEQIQAELEKKGYTVKLVEFTDYVRPNLALAEGELDINVFQHKPYLDDFKKEHNLDITEAFQVPTAPLGLYPGKLKSLEEVKDGSTVSAPNDPSNFARALVMLNELGWIKLKDGINPLTASKADIAENLKNIKIVELEAAQLPRSRADVDFAVVNGNYAISSGMKLTEALFQEPSFAYVAWSAVKTADKDSQWLKDVTEAYNSDAFKAYAHKRFEGYKYPAAWNEGAAK

SEQ ID NO: 4 - DNA, 1091 bp, synthetic construct
  misc_feature 1..1091: note = Neisseria gonorrhoeae MetQSM DNA Sequence
  source 1..1091: mol_type = other DNA
atgcatccgggcagcagcgcatcggctcgcacgaggtctgcgcttgaattgtgttgtagaaacacaacgtttttgaaaaaataagctattgttttatatcaaaatataatcatttttaaaataaaggttgcggcatttatcagatatttgttctgaaatgaagacgtatcgggtgtttgcccgatgtttttaggtttttatcaaatttacaaaaggaagcccatatgaaaaccttcttcaaaaccctttccgccgccgcactcgcgctcatcctcgcagcctgcggcggtcaaaaagacagcgcgcccgcagcctctgccgccgccccttctgccgataacggcgcggcgaaaaaagaaatcgtcttcggcacgaccgtcggcgacttcggcgatatggtcaaagaacaaatccaagccgagctggagaaaaaaggctacaccgtcaaattggtcgaatttaccgactatgtgcgcccgaatctggcattggcggagggcgagttggacatcaacgtcttccaacacaaaccctatcttgacgatttcaaaaaagaacacaacctggacatcaccgaagccttccaagtgccgaccgcgcctttgggactgtatccgggcaaactgaaatcgctggaagaagtcaaagacggcagcaccgtatccgcgcccaacgacccgtccaacttcgcacgcgccttggtgatgctgaacgaactgggttggatcaaactcaaagacggcatcaatccgctgaccgcatccaaagccgacatcgcggaaaacctgaaaaacatcaaaatcgtcgagcttgaagccgcacaactgccgcgcagccgcgccgacgtggattttgccgtcgtcaacggcaactacgccataagcagcggcatgaagctgaccgaagccctgttccaagagccgagctttgcctatgtcgcctggtctgccgtcaaaaccgccgacaaagacagccaatggcttaaagacgtaaccgaggcctataactccgacgcgttcaaagcctacgcgcacaaacgcttcgagggctacaaataccctgccgcatggaatgaaggcgcagccaaataa

SEQ ID NO: 5 - protein, 315 aa, synthetic construct
  REGION 1..315: note = Neisseria gonorrhoeae GNA1220 protein.
MEFFIILLAAVAVFGFKSFVVIPQQEVHVVERLGRFHRALTAGLNILIPFIDRVAYRHSLKEIPLDVPSQVCITRDNTQLTVDGIIYFQVTDPKLASYGSSNYIMAITQLAQTTLRSVIGRMELDKTFEERDEINSTVVSALDEAAGAWGVKVLRYEIKDLVPPQEILRAMQAQITAEREKRARIAESEGRKIEQINLASGQREAEIQQSEGEAQAAVNASNAEKIARINRAKGEAESLRLVAEANAEAIRQIAAALQTQGGADAVNLKIAEQYVAAFNNLAKESNTLIMPANVADIGSLISAGMKIIDSSKTAK

SEQ ID NO: 6 - DNA, 948 bp, synthetic construct
  misc_feature 1..948: note = Neisseria gonorrhoeae GNA1220 DNA sequence.
  source 1..948: mol_type = other DNA
atggaatttttcattatcttgttggcagccgtcgccgttttcggcttcaaatcctttgtcgtcatcccccagcaggaagtccacgttgtcgaaaggctcgggcgtttccatcgcgccctgacggccggtttgaatattttgattccctttatcgaccgcgtcgcctaccgccattcgctgaaagaaatccctttagacgtacccagccaggtctgcatcacgcgcgataatacgcaattgactgttgacggcatcatctatttccaagtaaccgatcccaaactcgcctcatacggttcgagcaactacattatggcaattacccagcttgcccaaacgacgctgcgttccgttatcgggcgtatggagttggacaaaacgtttgaagaacgcgacgaaatcaacagtaccgtcgtctccgccctcgatgaagccgccggggcttggggtgtgaaagtcctccgttacgaaatcaaggatttggttccgccgcaagaaatccttcgcgcaatgcaggcacaaattaccgccgaacgcgaaaaacgcgcccgtattgccgaatccgaaggccgtaaaatcgaacaaatcaaccttgccagtggtcagcgtgaagccgaaatccaacaatccgaaggcgaggctcaggctgcggtcaatgcgtccaatgccgagaaaatcgcccgcatcaaccgcgccaaaggcgaagcggaatccctgcgccttgttgccgaagccaatgccgaagccatccgtcaaattgccgccgcccttcaaacccaaggcggggcggatgcggtcaatctgaagattgcggaacaatacgtagccgcgttcaacaatcttgccaaagaaagcaatacgctgattatgcccgccaatgttgccgacatcggcagcctgatttctgccggcatgaaaattatcgacagcagcaaaaccgccaaataa

SEQ ID NO: 7 - protein, 148 aa, synthetic construct
  REGION 1..148: note = Neisseria gonorrhoeae GNA1220aba protein sequence.
MVNRTAFCLSLTAALILTACSSGGGGEQINLASGQREAEIQQSEGEAQAAVNASNAEKIARINRAKGEAESLRLVAEANAEAIRQIAAALQTQGGADAVNLKIAEQYVAAFNNLAKESNTLIMPANVADIGSLISAGMKIIDSSKTAK

SEQ ID NO: 8 - DNA, 447 bp, synthetic construct
  misc_feature 1..447: note = Neisseria gonorrhoeae GNA1220_helix-aba DNA sequence.
  source 1..447: mol_type = other DNA
atggtgaaccgaactgccttctgcctttctctgaccgccgccctgattctgaccgcctgcagcagcggagggggtggtgaacaaatcaaccttgccagtggtcagcgtgaagccgaaatccaacaatccgaaggcgaggctcaggctgcggtcaatgcgtccaatgccgagaaaatcgcccgcatcaaccgcgccaaaggcgaagcggaatccctgcgccttgttgccgaagccaatgccgaagccatccgtcaaattgccgccgcccttcaaacccaaggcggggcggatgcggtcaatctgaagattgcggaacaatacgtagccgcgttcaacaatcttgccaaagaaagcaatacgctgattatgcccgccaatgttgccgacatcggcagcctgatttctgccggcatgaaaattatcgacagcagcaaaaccgccaaataa

SEQ ID NO: 9 - DNA, 25 bp, synthetic construct
  misc_feature 1..25: note = MetQ_neisseria forward primer
  source 1..25: mol_type = other DNA
atacaattgcctcagcgcatgcatc

SEQ ID NO: 10 - DNA, 31 bp, synthetic construct
  misc_feature 1..31: note = MetQ_SbfI reverse primer
  source 1..31: mol_type = other DNA
tatcctgcaggttatacgactgccttatttg

SEQ ID NO: 11 - DNA, 31 bp, synthetic construct
  misc_feature 1..31: note = MetQ_neisseria reverse primer
  source 1..31: mol_type = other DNA
tattctagattatacgactgccttatttggc

SEQ ID NO: 12 - DNA, 33 bp, synthetic construct
  misc_feature 1..33: note = MetQ_SpeI reverse primer
  source 1..33: mol_type = other DNA
tatactagtttatacgactgccttatttggctg

SEQ ID NO: 13 - DNA, 30 bp, synthetic construct
  misc_feature 1..30: note = GNA1220_StuI reverse primer
  source 1..30: mol_type = other DNA
tataggccttatacgactgccttatttggc

SEQ ID NO: 14 - DNA, 2260 bp, synthetic construct
  misc_feature 1..2260: note = Blue script Plasmid (FHbp KO+MetQ)
  source 1..2260: mol_type = other DNA
atggcactcgtatccatgcgccaactgcttgatcatgctgccgaaaacagctacggcctgccggcgttcaacgtcaacaacctcgaacagatgcgcgccatcatggaggctgcagaccaagtcgacgcccccgtcatcgtacaggcgagtgccggtgcgcgcaaatatgcgggtgcgccgtttttacgccacctgattttggcggctgtcgaagaatttccacacatcccgtcgtcatgcaccaagaccacggcgcatcacccgacgtgtgccaacgctccatccaactgggcttctcctctgtaatgatggacggctcgctgatggaagacggcaaaaccccttcttcttacgaatacaacgtcaacgccacacgtaccgtggttaacttctcccacgcttgcggcgtatccgttgaaggcgaaatcggcgtattgggcaacctcgaaaccggcgaagcaggcgaagaagacggtgtaggcgcagtgggcaaactttcccacgaccaaatgctgaccagcgtcgaagatgccgtatgtttcgttaaagataccggcgttgacgcattggctattgccgtcggcaccagccacggcgcatacaaattcacccgtccgcccacaggcgatgtattacgtatcgaccgcatcaaagaaatccaccaagccctgcccaatacacacatcgtgatgcacggctccagctccgttccgcaagaatggctgaaagtcatcaacgaatacggcggcaatatcggcgaaacctacggcgtgccggttgaagaaatcgtcgaaggcatcaaacacggcgtgcgcaaagtcaacatcgataccgacttgcgccttgcttctaccggcgcggtacgccgctaccttgccgaaaatccgtccgactttgacccgcgcaaatacctgagcaaaaccattgaggccatgaagcaaatctgcctcgaccgttatcttgcgtttggctgcgaaggtcaggcaggcaaaatcaaacctgtttcgttggaaaaaatggcaagccgttatgccaagggcgaattgaaccaaatcgtcaaataacaggttgcctgtaaacaaaatgccgtcccatgggcaaacttaagagtgtgttgatagtgcagtatcttaaaattttgtgtataataggaattgaagttaaattagatgctaaaaatttgtaattaagaaggagggattcgtcatgttggtattccaaatgcgtaatgtagataaaacatctactgttttgaaacagactaaaaacagtgattacgcagataaataaatacgttagattaattcctaccagtgactaatcttatgactttttaaacagataactaaaattacaaacaaatcgtttaacttctgtatttatttacagatgtaatcacttcaggagtaattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaacattcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccattacttatgagcaagtattgtctatttttaatagttatctattatttaacgggaggaaataattctatgagtcgcttttttaaatttggaaagttacacgttactaaagggaatggagataaattattagatatactactgacagcttccaagaagctaaagaggtccct

SEQ ID NO: 15 - DNA, 20 bp, synthetic construct
  misc_feature 1..20: note = metQ pBS downstream forward primer
  source 1..20: mol_type = other DNA
ccctgttccaagagccgagc

SEQ ID NO: 16 - DNA, 20 bp, synthetic construct
  misc_feature 1..20: note = RBD pBS downstream reverse primer
  source 1..20: mol_type = other DNA
agcttcttccagcgcgaacg

SEQ ID NO: 17 - DNA, 20 bp, synthetic construct
  misc_feature 1..20: note = FHbp upstream forward primer
  source 1..20: mol_type = other DNA
ggcgaaatcggcgtattggg

SEQ ID NO: 18 - DNA, 23 bp, synthetic construct
  misc_feature 1..23: note = FHbp upstream reverse primer
  source 1..23: mol_type = other DNA
ctacattacgcatttggaatacc

SEQ ID NO: 19 - DNA, 5330 bp, synthetic construct
  misc_feature 1..5330: note = pGEM Plasmid (Capsule KO+MetQ)
  source 1..5330: mol_type = other DNA
ctattttccagtacctaaacgttccaaacgatagctgccgttcaatacattttgccaccaggttttgttgtccagataccattgcacggttttgcggaggccggactcgaaggtttccaaaggcagccagcccaaatcccgcctgattttggctgcgtcgacggcgtagcgtacgtcatggccggggcggtcttgtacgaaagtaatcaaatcttcataacgcgccacaccggccggtttttcgggagcgagttcttccagcagggcgcagatggttttgacgacttcaatattggctttttcattgtggccgccgatattgtaggtttcgccgacaacaccttcggtaacaacctgatacagtgcgcgcgcgtggtcttcgacaaacagccagtcgcggatttgcataccgtcgccgtacacaggcagcggtttgccgtcaagcgcgttcagaatcatcaaaggaatgagtttttccggaaaatggtaaggaccgtagttgttggagcagttggttacaatggtcggcaagccgtaagtacgcaaccacgcgcggacgaggtggtcgctggacactttagaggcagagtaggggctggacggcgcgtagggcgcggtttcggtaaacaaatcgtccgtgccgcctaaatcgccatagacttcatcggtggaaatatggtggaaacggaaggcttcgtgctgttcagacggcatttgttgccagtaggcgcgggctgcttcaagcagattgaatgtgccgacgatattggtttggataaactcgcctgccgaaccgatagagcggtcgacatggctttccgccgccaagtgcatcacggcatcgggccggtgttgtgcgaatacgcggtcgagttcggcgcggtcgcaaatgtccacttgctcgaaagcatagcggggattatcggcaatgtcggtcagcgattcgagattgcccgcgtaggtcagtttgtcgaggttgacgacggaatcttgggtgtttcggataatatgacggacaacggcagaaccgataaagcccgcaccgccggtaacgaggatggtttttttgtttgcggtttgcatttaatccccatatccgttggggttgcggctgacccaacgccacgaatcttccatcatttgctgcaagccgcgtttggtttcccagccggtttgttgtttggtatgggacgggtcggcaaaggaacacgccaaatcaccggcgcggcgcggtttgatttcaaatggaatggttaaacccgaagccgcttcaaatgcgcggatgatttccaataccgaagaagcgcggccggagcctaagttcagcaaatgcgtgcctgctacattactttttgcctgcatagccgcgacatggccttctgccaaatccatcacatgaatatagtcacgcatccccgtgccgtcgggggtagggtagtcatcgccaaataccgccaattgcctcagcgcatgcatccgggcagcagcgcatcggctcgcacgaggtctgcgcttgaattgtgttgtagaaacacaacgtttttgaaaaaataagctattgttttatatcaaaatataatcatttttaaaataaaggttgcggcatttatcagatatttgttctgaaatgaagacgtatcgggtgtttgcccgatgtttttaggtttttatcaaatttacaaaaggaagcccatatgaaaaccttcttcaaaaccctttccgccgccgcactcgcgctcatcctcgcagcctgcggcggtcaaaaagacagcgcgcccgcagcctctgccgccgccccttctgccgataacggcgcggcgaaaaaagaaatcgtcttcggcacgaccgtcggcgacttcggcgatatggtcaaagaacaaatccaagccgagctggagaaaaaaggctacaccgtcaaattggtcgaatttaccgactatgtgcgcccgaatctggcattggcggagggcgagttggacatcaacgtcttccaacacaaaccctatcttgacgatttcaaaaaagaacacaacctggacatcaccgaagccttccaagtgccgaccgcgcctttgggactgtatccgggcaaactgaaatcgctggaagaagtcaaagacggcagcaccgtatccgcgcccaacgacccgtccaacttcgcacgcgccttggtgatgctgaacgaactgggttggatcaaactcaaagacggcatcaatccgctgaccgcatccaaagccgacatcgcggaaaacctgaaaaacatcaaaatcgtcgagcttgaagccgcacaactgccgcgcagccgcgccgacgtggattttgccgtcgtcaacggcaactacgccataagcagcggcatgaagctgaccgaagccctgttccaagagccgagctttgcctatgtcaactggtctgccgtcaaaaccgccgacaaagacagccaatggcttaaagacgtaaccgaggcctataactccgacgcgttcaaagcctacgcgcacaaacgcttcgagggctacaaataccctgccgcatggaatgaaggcgcagccaaataaggcagtcgtataatctagagacgatgccgtctgaagaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctatgccgtctgaatactcgagcggaacccgggtagtggcctgtaaaagacagcatatagagatgagcaggctgtataatattaaggatttttttgtaacttctataaatataaagtaattttttaggagttatattattagggcttctaggaagctcaaatagataaatagattcaaatagattcttgttagctgattgatgaactaacttaggcatttttaagtttttagaagtatataaaattactagtaaattattggttaatttttgtattttaattaggctttggacttggttaagctgacctaaattagatatgacaaataaattgttacgtgggggggtaagataaaatggagatgttgtcaaccacattgaatcttgaaaaaactttttaggctgaaaaagagctttttttattttctttagcattattgtatctcttaaaaattaatgagaattagctatatgtaatagccaatcctctgttaataaagtaactaagttaataagcattattcaatatcagtttttttgatttgagcacctttgcgaatattgcaagcagcgaccttaccaaataatgtttcatattcgttgacgctgaagtctccattgcctgggcgtttaacccataggttatctccggacaacagttctccttttttaatgtctttatctgctacgacagatgcaaaggcgaaatctttagttggcttttctcccgcgataatcgtgtcttttttgccgccgcgtgccaattttaaagcatgagcgccttgcttgagctctttaaaagtatccggattcatagagcatacaatatccggacctgggcgatccatgcggtcagtaaagtgacgctctaaaatcgaaccgcctaaagctactgctcctaagcaagcatagttatctaaggtatggtcagacaggccaatgattgcgtctggaaaggcttcagataaatcgttcataccacccaatcgaacatcttcgtaaggggttgggtagatgttggtacagtgaagcaaagcataaggtacccctgcttctcgaataatttctaccgactttttgatgctttcaatagaattcatgccggtagagagaataataggcttaccaaaagaggccaccagtttaattaatgggtagttattacattcgccagagccgattttatatgctggaatatccatacgttgtaatcgtaaagcagctgcacgagagaaaggagtactgataaaaatcatacccttactctctacgtattcttttaatttaatctcatcttcttcattcagggcgcaacgttccataatttcataaatagagacatctgcattgcctggaatgacttgtttggcctcatcagacatttcgtcttcaacgatgtgtgtttgatgtttaacaacttcagcgcctgcattataggcagcatcaaccatttcaaaagctgtttttaaagagccttcatgattgatgccgatttcacagataatcaatggttcgtggttgtaacctactgaacgattaccaattttaaattcgttgttgttttgcat

SEQ ID NO: 20 - DNA, 20 bp, synthetic construct
  misc_feature 1..20: note = Capsule KO GalE forward primer
  source 1..20: mol_type = other DNA
atacgcggtcgagttcggcg

SEQ ID NO: 21 - DNA, 20 bp, synthetic construct
  misc_feature 1..20: note = Capsule KO upstream metQ Reverse primer
  source 1..20: mol_type = other DNA
gcgcgctgtctttttgaccg

SEQ ID NO: 22 - DNA, 24 bp, synthetic construct
  misc_feature 1..24: note = Capsule KO Spc downstream forward primer
  source 1..24: mol_type = other DNA
gatgagcgaaatgtagtgcttacg

SEQ ID NO: 23 - DNA, 24 bp, synthetic construct
  misc_feature 1..24: note = Capsule KO SiaD reverse primer
  source 1..24: mol_type = other DNA
gcatctgtcgtagcagataaagac

SEQ ID NO: 24 - DNA, 3392 bp, synthetic construct
  misc_feature 1..3392: note = pUC18 Plasmid (lpxL1 KO+MetQ)
  source 1..3392: mol_type = other DNA
atgtcggcaaccgtccgtatcttgggcattgacccgggcagccgcgtaacgggtttcggcatcatcgatgtcagggggcgcgatcatttttacgttgcctcaggctgcatcaaaacgcctgccgatgcgcctctggcagacaggattgccgtcatcgtccggcacatcggcgaagtcgttaccgtttacaagcctcaacaggcggcggtggaacaggtgttcgtcaacgtcaatcctgcatcgacgctgatgctcggtcaggcgcggggcgcggcattggcggcattggtcagccataagctgcccgtttcggaatacacggccttgcaggtcaaacaggcggtggtcggcaagggtaaggcagcgaaagaacaggtgcagcatatggtggtgcagatgctggggctttcgggaacaccgcaggcggatgcggcggacggtcttgccgtcgcgctgacccacgccttacgcaaccacgggcttgccgccaaactcaatccttcagggatgcaggtcaagcgcggaaggtttcaatagtttcagacggcatttgtattttgccgtctgaaaagaaaatgtgtatcgagatgaaatttatatttttgtactgtatgttttgcagtttctgccgtttgcgctgctgcacaagattgccgacctgacgggtttgcttgcctaccttctggtcaaaccgcgccgccgtatcggcgaaatcaatttggcaaaatgtttttccgaatggagtgaggaaaagcgtaaaaccgtgttgaaacagcatttcaaacacatggcgaaactgatgttggaatacggtttatattggtacgcgcctgccggacgtttgaaatcgctggtgcgctaccgcaataagcattatttggacgacgcgctggcggcgggggaaaaagtcatcatcctgtatccgcacttcaccgctgcagttgcagtgactaactaggaggaataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacccctcagcgcatgcatccgggctctgcgcttgaattgtgttgtagaaacacaacgtttttgaaaaaataagctattgttttatatcaaaatataatcatttttaaaataaaggttgcggcatttatcagatatttgttctgaaatgaagacgtatcgggtgtttgcccgatgtttttaggtttttatcaaatttacaaaaggaagcccatatgaaaaccttcttcaaaaccctttccgccgccgcactcgcgctcatcctcgcagcctgcggcggtcaaaaagacagcgcgcccgcagcctctgccgccgccccttctgccgataacggcgcggcgaaaaaagaaatcgtcttcggcacgaccgtcggcgacttcggcgatatggtcaaagaacaaatccaagccgagctggagaaaaaaggctacaccgtcaaattggtcgaatttaccgactatgtgcgcccgaatctggcattggcggagggcgagttggacatcaacgtcttccaacacaaaccctatcttgacgatttcaaaaaagaacacaacctggacatcaccgaagccttccaagtgccgaccgcgcctttgggactgtatccgggcaaactgaaatcgctggaagaagtcaaagacggcagcaccgtatccgcgcccaacgacccgtccaacttcgcacgcgccttggtgatgctgaacgaactgggttggatcaaactcaaagacggcatcaatccgctgaccgcatccaaagccgacatcgcggaaaacctgaaaaacatcaaaatcgtcgagcttgaagccgcacaactgccgcgcagccgcgccgacgtggattttgccgtcgtcaacggcaactacgccataagcagcggcatgaagctgaccgaagccctgttccaagagccgagctttgcctatgtcaactggtctgccgtcaaaaccgccgacaaagacagccaatggcttaaagacgtaaccgaggcctataactccgacgcgttcaaagcctacgcgcacaaacgcttcgagggctacaaataccctgccgcatggaatgaaggcgcagccaaataaggcagtcgtataacctgcagggaataatgagtcgacaggatttcggacgcaacgattcggtttttgtggattttttcggtattcagacggcaacgattaccggattgagccgcattgccgcgcttgcaaatgcaaaagtgatacccgccattcccgtccgcgaggcagacaatacggttacattgcatttctaccctgcttggaaatcctttccgggtgaagacgcgaaagccgacgcgcagcgcatgaaccgttttatcgaagacagggtgcgcgaacatccggaacaatatttttggctgcacaagcgttttaaaacccgtccggaaggcagccccgatttttactgactacgtaaaattacaaaacatatcaggcgtttcgagtcgaaactcctgattgtttttttaaatgcaaaatggcagattatatgaacagatttcatttgataatcgctgctatttaagtatctcaaaaaacaatatttttaagacttggtcgggaaattcgaagcaggttttggaagaaataccgcaaacctatgaatata

SEQ ID NO: 25 - DNA, 20 bp, synthetic construct
  misc_feature 1..20: note = Lpxl1 upstream Forward primer
  source 1..20: mol_type = other DNA
gtcggcaagggtaaggcagc

SEQ ID NO: 26 - DNA, 20 bp, synthetic construct
  misc_feature 1..20: note = Lpxl1 upstream reverse primer
  source 1..20: mol_type = other DNA
ccctttataccggctgtccg

SEQ ID NO: 27 - DNA, 24 bp, synthetic construct
  misc_feature 1..24: note = Lpxl1 downstream reverse primer
  source 1..24: mol_type = other DNA
atattcataggtttgcggtatttc

SEQ ID NO: 28 - DNA, 2261 bp, synthetic construct
  misc_feature 1..2261: note = Blue script Plasmid (FHbp KO+GNA1220)
  source 1..2261: mol_type = other DNA
atggcactcgtatccatgcgccaactgcttgatcatgctgccgaaaacagctacggcctgccggcgttcaacgtcaacaacctcgaacagatgcgcgccatcatggaggctgcagaccaagtcgacgcccccgtcatcgtacaggcgagtgccggtgcgcgcaaatatgcgggtgcgccgtttttacgccacctgattttggcggctgtcgaagaatttccacacatccccgtcgtcatgcaccaagaccacggcgcatcacccgacgtgtgccaacgctccatccaactgggcttctcctctgtaatgatggacggctcgctgatggaagacggcaaaaccccttcttcttacgaatacaacgtcaacgccacacgtaccgtggttaacttctcccacgcttgcggcgtatccgttgaaggcgaaatcggcgtattgggcaacctcgaaaccggcgaagcaggcgaagaagacggtgtaggcgcagtgggcaaactttcccacgaccaaatgctgaccagcgtcgaagatgccgtatgtttcgttaaagataccggcgttgacgcattggctattgccgtcggcaccagccacggcgcatacaaattcacccgtccgcccacaggcgatgtattacgtatcgaccgcatcaaagaaatccaccaagccctgcccaatacacacatcgtgatgcacggctccagctccgttccgcaagaatggctgaaagtcatcaacgaatacggcggcaatatcggcgaaacctacggcgtgccggttgaagaaatcgtcgaaggcatcaaacacggcgtgcgcaaagtcaacatcgataccgacttgcgccttgcttctaccggcgcggtacgccgctaccttgccgaaaatccgtccgactttgacccgcgcaaatacctgagcaaaaccattgaggccatgaagcaaatctgcctcgaccgttatcttgcgtttggctgcgaaggtcaggcaggcaaaatcaaacctgtttcgttggaaaaaatggcaagccgttatgccaagggcgaattgaaccaaatcgtcaaataacaggttgcctgtaaacaaaatgccgtcccatgggcaaacttaagagtgtgttgatagtgcagtatcttaaaattttgtgtataataggaattgaagttaaattagatgctaaaaatttgtaattaagaaggagggattcgtcatgttggtattccaaatgcgtaatgtagataaaacatctactgttttgaaacagactaaaaacagtgattacgcagataaataaatacgttagattaattcctaccagtgactaatcttatgactttttaaacagataactaaaattacaaacaaatcgtttaacttctgtatttatttacagatgtaatcacttcaggagtaattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaacattcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccattacttatgagcaagtattgtctatttttaatagttatctattatttaacgggaggaaataattctatgagtcgcttttttaaatttggaaagttacacgttactaaagggaatggagataaattattagatatactactgacagcttccaagaagctaaagaggtccct

SEQ ID NO: 29 - DNA, 24 bp, synthetic construct
  misc_feature 1..24: note = GNA1220 pBS downstream forward primer
  source 1..24: mol_type = other DNA
cttgccaaagaaagcaatacgctg

SEQ ID NO: 30 - DNA, 5411 bp, synthetic construct
  misc_feature 1..5411: note = pGEM Plasmid (Capsule KO+GNA1220)
  source 1..5411: mol_type = other DNA
ctattttccagtacctaaacgttccaaacgatagctgccgttcaatacattttgccaccaggttttgttgtccagataccattgcacggttttgcggaggccggactcgaaggtttccaaaggcagccagcccaaatcccgcctgattttggctgcgtcgacggcgtagcgtacgtcatggccggggcggtcttgtacgaaagtaatcaaatcttcataacgcgccacaccggccggtttttcgggagcgagttcttccagcagggcgcagatggttttgacgacttcaatattggctttttcattgtggccgccgatattgtaggtttcgccgacaacaccttcggtaacaacctgatacagtgcgcgcgcgtggtcttcgacaaacagccagtcgcggatttgcataccgtcgccgtacacaggcagcggtttgccgtcaagcgcgttcagaatcatcaaaggaatgagtttttccggaaaatggtaaggaccgtagttgttggagcagttggttacaatggtcggcaagccgtaagtacgcaaccacgcgcggacgaggtggtcgctggacactttagaggcagagtaggggctggacggcgcgtagggcgcggtttcggtaaacaaatcgtccgtgccgcctaaatcgccatagacttcatcggtggaaatatggtggaaacggaaggcttcgtgctgttcagacggcatttgttgccagtaggcgcgggctgcttcaagcagattgaatgtgccgacgatattggtttggataaactcgcctgccgaaccgatagagcggtcgacatggctttccgccgccaagtgcatcacggcatcgggccggtgttgtgcgaatacgcggtcgagttcggcgcggtcgcaaatgtccacttgctcgaaagcatagcggggattatcggcaatgtcggtcagcgattcgagattgcccgcgtaggtcagtttgtcgaggttgacgacggaatcttgggtgtttcggataatatgacggacaacggcagaaccgataaagcccgcaccgccggtaacgaggatggtttttttgtttgcggtttgcatttaatccccatatccgttggggttgcggctgacccaacgccacgaatcttccatcatttgctgcaagccgcgtttggtttcccagccggtttgttgtttggtatgggacgggtcggcaaaggaacacgccaaatcaccggcgcggcgcggtttgatttcaaatggaatggttaaacccgaagccgcttcaaatgcgcggatgatttccaataccgaagaagcgcggccggagcctaagttcagcaaatgcgtgcctgctacattactttttgcctgcatagccgcgacatggccttctgccaaatccatcacatgaatatagtcacgcatccccgtgccgtcgggggtagggtagtcatcgccaaataccgccaattgcctcagcgcatgcatccgggcagcagcgcatcggctcgcacgaggtctgcgcttgaattgtgttgtagaaacacaacgtttttgaaaaaataagctattgttttatatcaaaatataatcatttttaaaataaaggttgcggcatttatcagatatttgttctgaaatgaagacgtatcgggtgtttgcccgatgtttttaggtttttatcaaatttacaaaaggaagcccatatggaatttttcattatcttgttggcagccgtcgccgttttcggcttcaaatcctttgtcgtcatcccccagcaggaagtccacgttgtcgaaaggctcgggcgtttccatcgcgccctgacggccggtttgaatattttgattccctttatcgaccgcgtcgcctaccgccattcgctgaaagaaatccctttagacgtacccagccaggtctgcatcacgcgcgataatacgcaattgactgttgacggcatcatctatttccaagtaaccgatcccaaactcgcctcatacggttcgagcaactacattatggcaattacccagcttgcccaaacgacgctgcgttccgttatcgggcgtatggagttggacaaaacgtttgaagaacgcgacgaaatcaacagtaccgtcgtctccgccctcgatgaagccgccggggcttggggtgtgaaagtcctccgttacgaaatcaaggatttggttccgccgcaagaaatccttcgcgcaatgcaggcacaaattaccgccgaacgcgaaaaacgcgcccgtattgccgaatccgaaggccgtaaaatcgaacaaatcaaccttgccagtggtcagcgtgaagccgaaatccaacaatccgaaggcgaggctcaggctgcggtcaatgcgtccaatgccgagaaaatcgcccgcatcaaccgcgccaaaggcgaagcggaatccctgcgccttgttgccgaagccaatgccgaagccatccgtcaaattgccgccgcccttcaaacccaaggcggggcggatgcggtcaatctgaagattgcggaacaatacgtagccgcgttcaacaatcttgccaaagaaagcaatacgctgattatgcccgccaatgttgccgacatcggcagcctgatttctgccggcatgaaaattatcgacagcagcaaaaccgccaaataaggcagtcgtataatctagagacgatgccgtctgaagaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctatgccgtctgaatactcgagcggaacccgggtagtggcctgtaaaagacagcatatagagatgagcaggctgtataatattaaggatttttttgtaacttctataaatataaagtaattttttaggagttatattattagggcttctaggaagctcaaatagataaatagattcaaatagattcttgttagctgattgatgaactaacttaggcatttttaagtttttagaagtatataaaattactagtaaattattggttaatttttgtattttaattaggctttggacttggttaagctgacctaaattagatatgacaaataaattgttacgtgggggggtaagataaaatggagatgttgtcaaccacattgaatcttgaaaaaactttttaggctgaaaaagagctttttttattttctttagcattattgtatctcttaaaaattaatgagaattagctatatgtaatagccaatcctctgttaataaagtaactaagttaataagcattattcaatatcagtttttttgatttgagcacctttgcgaatattgcaagcagcgaccttaccaaataatgtttcatattcgttgacgctgaagtctccattgcctgggcgtttaacccataggttatctccggacaacagttctccttttttaatgtctttatctgctacgacagatgcaaaggcgaaatctttagttggcttttctcccgcgataatcgtgtcttttttgccgccgcgtgccaattttaaagcatgagcgccttgcttgagctctttaaaagtatccggattcatagagcatacaatatccggacctgggcgatccatgcggtcagtaaagtgacgctctaaaatcgaaccgcctaaagctactgctcctaagcaagcatagttatctaaggtatggtcagacaggccaatgattgcgtctggaaaggcttcagataaatcgttcataccacccaatcgaacatcttcgtaaggggttgggtagatgttggtacagtgaagcaaagcataaggtacccctgcttctcgaataatttctaccgactttttgatgctttcaatagaattcatgccggtagagagaataataggcttaccaaaagaggccaccagtttaattaatgggtagttattacattcgccagagccgattttatatgctggaatatccatacgttgtaatcgtaaagcagctgcacgagagaaaggagtactgataaaaatcatacccttactctctacgtattcttttaatttaatctcatcttcttcattcagggcgcaacgttccataatttcataaatagagacatctgcattgcctggaatgacttgtttggcctcatcagacatttcgtcttcaacgatgtgtgtttgatgtttaacaacttcagcgcctgcattataggcagcatcaaccatttcaaaagctgtttttaaagagccttcatgattgatgccgatttcacagataatcaatggttcgtggttgtaacctactgaacgattaccaattttaaattcgttgttgttttgcat

SEQ ID NO: 31 - DNA, 20 bp, synthetic construct
  misc_feature 1..20: note = Capsule KO upstream GNA1220 R
  source 1..20: mol_type = other DNA
tgaagccgaaaacggcgacg

SEQ ID NO: 32 - DNA, 3498 bp, synthetic construct
  misc_feature 1..3498: note = pUC18 Plasmid (lpxL1 KO+GNA1220)
  source 1..3498: mol_type = other DNA
atgtcggcaaccgtccgtatcttgggcattgacccgggcagccgcgtaacgggtttcggcatcatcgatgtcagggggcgcgatcatttttacgttgcctcaggctgcatcaaaacgcctgccgatgcgcctctggcagacaggattgccgtcatcgtccggcacatcggcgaagtcgttaccgtttacaagcctcaacaggcggcggtggaacaggtgttcgtcaacgtcaatcctgcatcgacgctgatgctcggtcaggcgcggggcgcggcattggcggcattggtcagccataagctgcccgtttcggaatacacggccttgcaggtcaaacaggcggtggtcggcaagggtaaggcagcgaaagaacaggtgcagcatatggtggtgcagatgctggggctttcgggaacaccgcaggcggatgcggcggacggtcttgccgtcgcgctgacccacgccttacgcaaccacgggcttgccgccaaactcaatccttcagggatgcaggtcaagcgcggaaggtttcaatagtttcagacggcatttgtattttgccgtctgaaaagaaaatgtgtatcgagatgaaatttatattttttgtactgtatgttttgcagtttctgccgtttgcgctgctgcacaagattgccgacctgacgggtttgcttgcctaccttctggtcaaaccgcgccgccgtatcggcgaaatcaatttggcaaaatgtttttccgaatggagtgaggaaaagcgtaaaaccgtgttgaaacagcatttcaaacacatggcgaaactgatgttggaatacggtttatattggtacgcgcctgccggacgtttgaaatcgctggtgcgctaccgcaataagcattatttggacgacgcgctggcggcgggggaaaaagtcatcatcctgtatccgcacttcaccgctgcagttgcagtgactaactaggaggaataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacccctcagcgcatgcatccgggcagcagcgcatcggctcgcacgaggtctgcgcttgaattgtgttgtagaaacacaacgtttttgaaaaaataagctattgttttatatcaaaatataatcatttttaaaataaaggttgcggcatttatcagatatttgttctgaaatgaagacgtatcgggtgtttgcccgatgtttttaggtttttatcaaatttacaaaaggaagcccatatggaatttttcattatcttgttggcagccgtcgccgttttcggcttcaaatcctttgtcgtcatcccccagcaggaagtccacgttgtcgaaaggctcgggcgtttccatcgcgccctgacggccggtttgaatattttgattccctttatcgaccgcgtcgcctaccgccattcgctgaaagaaatccctttagacgtacccagccaggtctgcatcacgcgcgataatacgcaattgactgttgacggcatcatctatttccaagtaaccgatcccaaactcgcctcatacggttcgagcaactacattatggcaattacccagcttgcccaaacgacgctgcgttccgttatcgggcgtatggagttggacaaaacgtttgaagaacgcgacgaaatcaacagtaccgtcgtctccgccctcgatgaagccgccggggcttggggtgtgaaagtcctccgttacgaaatcaaggatttggttccgccgcaagaaatccttcgcgcaatgcaggcacaaattaccgccgaacgcgaaaaacgcgcccgtattgccgaatccgaaggccgtaaaatcgaacaaatcaaccttgccagtggtcagcgtgaagccgaaatccaacaatccgaaggcgaggctcaggctgcggtcaatgcgtccaatgccgagaaaatcgcccgcatcaaccgcgccaaaggcgaagcggaatccctgcgccttgttgccgaagccaatgccgaagccatccgtcaaattgccgccgcccttcaaacccaaggcggggcggatgcggtcaatctgaagattgcggaacaatacgtagccgcgttcaacaatcttgccaaagaaagcaatacgctgattatgcccgccaatgttgccgacatcggcagcctgatttctgccggcatgaaaattatcgacagcagcaaaaccgccaaataaggcagtcgtataacctgcagggaataatgagtcgacaggatttcggacgcaacgattcggtttttgtggattttttcggtattcagacggcaacgattaccggattgagccgcattgccgcgcttgcaaatgcaaaagtgatacccgccattcccgtccgcgaggcagacaatacggttacattgcatttctaccctgcttggaaatcctttccgggtgaagacgcgaaagccgacgcgcagcgcatgaaccgttttatcgaagacagggtgcgcgaacatccggaacaatatttttggctgcacaagcgttttaaaacccgtccggaaggcagccccgatttttactgactacgtaaaattacaaaacatatcaggcgtttcgagtcgaaactcctgattgtttttttaaatgcaaaatggcagattatatgaacagatttcatttgataatcgctgctatttaagtatctcaaaaaacaatatttttaagacttggtcgggaaattcgaagcaggttttggaagaaataccgcaaacctatgaatata

SEQ ID NO: 33 - DNA, 6740 bp, synthetic construct
  misc_feature 1..6740: note = pFP12-MetQ plasmid
  source 1..6740: mol_type = other DNA
ctgcaggccgtctgaaccgctctaaccgctttttctcggcttaatttttctgtctctgttataaaattgctattcatcttgttcttcttcaaaaaaaagttaagtaaaatacctacctaaatttttactagttcgcaatctacgagcttataacctcgttttttcaattcatttaaaaaatcagattttgagcctaattttgatctattgctatcgttacccgctagaaatacccagtaattacgcaaatcttcattggtaactttcgtaatatcggtgtaatgatcttcgagtatttttaagcaatctctagcccataaaccgtactcgtgattgctcatcttagggttttgcttatcgagtttgacgaacttcccatacttgtttttatgtggaaatactggccgttttgcaacttcttcaattttttgagctgttcgttttttactaccaatcacaaaatttaaagagtgaatagtacgcccacgcttgatttgttcaacctcaacgactaaatcagatttctcgttaatctcagttattgcaggttccaaaacacgttgatttaatgaattaaatctagggtatttattttcaacctgaagccattcttttagtttttctactgtaatttcacgactaccaacagagcgatattgtgtaattagctcataaattcgaattgaatgtacactgttgaaataagcgatatgtttgagttgatattgcgtgaattgccctttaagttgcgttaggtatggcataacttcatcagtcattgcaattctaaaacgcccctctttcttgaaatatgttctagaggaaacccaacgaaattcagttacacggtctttatcttcagttttaacacttcggtcataaatccgttttatagccgcctgaatttgcttataggcgttatcttggcttatttctggaaactcacggacaaaatcagccaccgtaaaatcaaaaatcttttgattagatttcggatccatagtcccaatagttaaagctaaaattctgatttcatcaatactcaatcggtaattggcttcaataaggctattagcctttacaacaactaaatcatttggcataagacaacaaatttcctgtttaaaacaacaagcaaaatatacctgttgtttatatataaaacaacaagtattttcttaaaagttgtctataacaggaaatttgttgtcttataacaggaaatttgttgtcgtataacaggaaatttgttgtcgtataacaggaaatttgttgtcgtataagtttgtaacttattgattttactggttttaaaaacgccgaaaacaagtaaaaaacaaaaatataaaaatatagggactttcgtcccttttttgggctttcagccctaattttttctttttttcaggatttaaaattacaaaacccttacagagcaagtaaacttgtttgcttgttctgcaagggttcagcaaccgtagccgtcaggcgtagggcggtagcctataaaagccatttaattttatctttaaacttccttttaaatgctttgagtgggtgtcttttatcgtactcatcaatccttttttgcattctttcgtttgctttgtgatcggcaaattttgaataagatttttccatctcatctaacattctatcaatccgttttttatgttgccatttcaggtaaacataaacacttatagcaattaaagacaatatcaatacattgtaaaaaatgattgttacaatttcgctcacagttattttttacctttttcaatttcttcattgataaatgcactcaattcatcaaatttcttgtcatcattgataaatttacgcaacttagggaagtttctatctacatctaaaagagggttatttattatttcatttagccaaaaagcccctaataaaaccttgtaatgcgtagctttcttacgcttttctgcttgttcttttgacttaatcgcacgaattttcgctttgatttcgtcctgcttgcgttgtaaatctgcttgttgctgttccaatcttgtaagtttttcgcttgccatactagcccctttatatagttagaaattatcgttattttattcagtaggtgctaggcttgcaagtgttctgttcattacgttaaaataacgtaatgcccacttatcagtttctcttcgagaaactggtgggcaagcgtaccgcttgaccgtttcgcaatactcaacactatggcaatctatcatttaaacgttcgctattgcagtaaaagcaaagggcaatcagctcaagccaaaaacgactacatcaaccgcaatgataaatattcaaagcggttagatgatttacagttttcaggctatggtaatatgccaaaatttgccgaagataatccgcaagaattttggcgattgtcagatatttacgagcgagctaatgcccgagtttgtactgaaattgaatttgctttacctagagaattaaccctagaacaacagcaaaaattagtaagttcgtttatagaaaatacggttgatagcggtagcaataaactaccctactctttcgctatccataccgataaaaataatcataatccccattgtcatttgatattttcagaacgccaacttgacggcatagaccgtacagccgagcagttttttaaacgtgctaatactaaatccccagaaaagggcggagcgatgaaaacggcagattttcgagatcgtgagtttatccaatctgtccgaaaaacgtggagagagcaagctaatcaagccttagagcaatacggatatgccgcacgaattgacgaacgtagctacaaggaacaaggcatagagcaagccccaagagcaagaattgacagggtaacgtggcaagaattgaaccgattagagcaagaagaacgccaaatcgtgcaagagcttgcacttaaaggacaagaaattaacaaagaaaaatcctacttgcagaaaatcgaagaaaaacaggctcaaggaatgggcaaatatgaatccaaattcgcagctgcgttttctaaattatcggaaagtgccctaaaacacgatttaagcaacgaaaaagaaaaagacagtaaaatacacactcaagaagaaaaagtgcctcaaaatcgcattcaggggctttctcaagcagattttgatcagtttttaattgatgaatggctacctcaaatagaaaaatacgttaaagcccaagaaaagcgggacggaatggaagtagagatcacgcaatacgacaaggatttacagcgtattcagggagactataacaagctcacagataaaaatcagggttttctcggtttatgggaaactaaagagcaaaaagcaaagaaaaaagagcttgaagatgaatacaaacatacagcagagcaacggaacgctaaaagccaagaattagccgagtatagccaaaaaataaaagcatacgaacagaaaacgctagagccaatcaacgagaagattgccaaatatcaagctgacaaccctgaaataaaaatgcggagcttaggatttgtgaaaaaaattaaggctcaaggggcatataaagcggctcaagagcgaatggagcgagaaaaacagcaccaacaggaaaaacaacagagacatttagagcgagagagtggtttgagcttgtagctaacgccctacgcctacggcttcggttgttcaacccttaaagaactcgcaacaagttgcaaattctttaagggttcgcaataaaaacaaccgctaaacatttctgcccagcggttgaaaatttacctattcaccattacaatgatcaagcaggaaatttttttgattgccgtaaatgtccgtatatctagttgaggcacaacccgccaaagtcattgccccaaccagaacggcgaagctaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttcctcagcgcatgcatccgggctctgcgcttgaattgtgttgtagaaacacaacgtttttgaaaaaataagctattgttttatatcaaaatataatcatttttaaaataaaggttgcggcatttatcagatatttgttctgaaatgaagacgtatcgggtgtttgcccgatgtttttaggtttttatcaaatttacaaaaggaagcccatatgaaaaccttcttcaaaaccctttccgccgccgcactcgcgctcatcctcgcagcctgcggcggtcaaaaagacagcgcgcccgcagcctctgccgccgccccttctgccgataacggcgcggcgaaaaaagaaatcgtcttcggcacgaccgtcggcgacttcggcgatatggtcaaagaacaaatccaagccgagctggagaaaaaaggctacaccgtcaaattggtcgaatttaccgactatgtgcgcccgaatctggcattggcggagggcgagttggacatcaacgtcttccaacacaaaccctatcttgacgatttcaaaaaagaacacaacctggacatcaccgaagccttccaagtgccgaccgcgcctttgggactgtatccgggcaaactgaaatcgctggaagaagtcaaagacggcagcaccgtatccgcgcccaacgacccgtccaacttcgcacgcgccttggtgatgctgaacgaactgggttggatcaaactcaaagacggcatcaatccgctgaccgcatccaaagccgacatcgcggaaaacctgaaaaacatcaaaatcgtcgagcttgaagccgcacaactgccgcgcagccgcgccgacgtggattttgccgtcgtcaacggcaactacgccataagcagcggcatgaagctgaccgaagccctgttccaagagccgagctttgcctatgtcaactggtctgccgtcaaaaccgccgacaaagacagccaatggcttaaagacgtaaccgaggcctataactccgacgcgttcaaagcctacgcgcacaaacgcttcgagggctacaaataccctgccgcatggaatgaaggcgcagccaaataagtgcggtcatcttcggtttccgtgtttcgtaaagtctggaaacgcggaagtcccctacgtgctgctgaagttgccccaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaaca

SEQ ID NO: 34 - DNA, 6740 bp, synthetic construct
  misc_feature 1..6740: note = pFP12-MetQSM plasmid
  source 1..6740: mol_type = other DNA
ctgcaggccgtctgaaccgctctaaccgctttttctcggcttaatttttctgtctctgttataaaattgctattcatcttgttcttcttcaaaaaaaagttaagtaaaatacctacctaaatttttactagttcgcaatctacgagcttataacctcgttttttcaattcatttaaaaaatcagattttgagcctaattttgatctattgctatcgttacccgctagaaatacccagtaattacgcaaatcttcattggtaactttcgtaatatcggtgtaatgatcttcgagtatttttaagcaatctctagcccataaaccgtactcgtgattgctcatcttagggttttgcttatcgagtttgacgaacttcccatacttgtttttatgtggaaatactggccgttttgcaacttcttcaattttttgagctgttcgttttttactaccaatcacaaaatttaaagagtgaatagtacgcccacgcttgatttgttcaacctcaacgactaaatcagatttctcgttaatctcagttattgcaggttccaaaacacgttgatttaatgaattaaatctagggtatttattttcaacctgaagccattcttttagtttttctactgtaatttcacgactaccaacagagcgatattgtgtaattagctcataaattcgaattgaatgtacactgttgaaataagcgatatgtttgagttgatattgcgtgaattgccctttaagttgcgttaggtatggcataacttcatcagtcattgcaattctaaaacgcccctctttcttgaaatatgttctagaggaaacccaacgaaattcagttacacggtctttatcttcagttttaacacttcggtcataaatccgttttatagccgcctgaatttgcttataggcgttatcttggcttatttctggaaactcacggacaaaatcagccaccgtaaaatcaaaaatcttttgattagatttcggatccatagtcccaatagttaaagctaaaattctgatttcatcaatactcaatcggtaattggcttcaataaggctattagcctttacaacaactaaatcatttggcataagacaacaaatttcctgtttaaaacaacaagcaaaatatacctgttgtttatatataaaacaacaagtattttcttaaaagttgtctataacaggaaatttgttgtcttataacaggaaatttgttgtcgtataacaggaaatttgttgtcgtataacaggaaatttgttgtcgtataagtttgtaacttattgattttactggttttaaaaacgccgaaaacaagtaaaaaacaaaaatataaaaatatagggactttcgtcccttttttgggctttcagccctaattttttctttttttcaggatttaaaattacaaaacccttacagagcaagtaaacttgtttgcttgttctgcaagggttcagcaaccgtagccgtcaggcgtagggcggtagcctataaaagccatttaattttatctttaaacttccttttaaatgctttgagtgggtgtcttttatcgtactcatcaatccttttttgcattctttcgtttgctttgtgatcggcaaattttgaataagatttttccatctcatctaacattctatcaatccgttttttatgttgccatttcaggtaaacataaacacttatagcaattaaagacaatatcaatacattgtaaaaaatgattgttacaatttcgctcacagttattttttacctttttcaatttcttcattgataaatgcactcaattcatcaaatttcttgtcatcattgataaatttacgcaacttagggaagtttctatctacatctaaaagagggttatttattatttcatttagccaaaaagcccctaataaaaccttgtaatgcgtagctttcttacgcttttctgcttgttcttttgacttaatcgcacgaattttcgctttgatttcgtcctgcttgcgttgtaaatctgcttgttgctgttccaatcttgtaagtttttcgcttgccatactagcccctttatatagttagaaattatcgttattttattcagtaggtgctaggcttgcaagtgttctgttcattacgttaaaataacgtaatgcccacttatcagtttctcttcgagaaactggtgggcaagcgtaccgcttgaccgtttcgcaatactcaacactatggcaatctatcatttaaacgttcgctattgcagtaaaagcaaagggcaatcagctcaagccaaaaacgactacatcaaccgcaatgataaatattcaaagcggttagatgatttacagttttcaggctatggtaatatgccaaaatttgccgaagataatccgcaagaattttggcgattgtcagatatttacgagcgagctaatgcccgagtttgtactgaaattgaatttgctttacctagagaattaaccctagaacaacagcaaaaattagtaagttcgtttatagaaaatacggttgatagcggtagcaataaactaccctactctttcgctatccataccgataaaaataatcataatccccattgtcatttgatattttcagaacgccaacttgacggcatagaccgtacagccgagcagttttttaaacgtgctaatactaaatccccagaaaagggcggagcgatgaaaacggcagattttcgagatcgtgagtttatccaatctgtccgaaaaacgtggagagagcaagctaatcaagccttagagcaatacggatatgccgcacgaattgacgaacgtagctacaaggaacaaggcatagagcaagccccaagagcaagaattgacagggtaacgtggcaagaattgaaccgattagagcaagaagaacgccaaatcgtgcaagagcttgcacttaaaggacaagaaattaacaaagaaaaatcctacttgcagaaaatcgaagaaaaacaggctcaaggaatgggcaaatatgaatccaaattcgcagctgcgttttctaaattatcggaaagtgccctaaaacacgatttaagcaacgaaaaagaaaaagacagtaaaatacacactcaagaagaaaaagtgcctcaaaatcgcattcaggggctttctcaagcagattttgatcagtttttaattgatgaatggctacctcaaatagaaaaatacgttaaagcccaagaaaagcgggacggaatggaagtagagatcacgcaatacgacaaggatttacagcgtattcagggagactataacaagctcacagataaaaatcagggttttctcggtttatgggaaactaaagagcaaaaagcaaagaaaaaagagcttgaagatgaatacaaacatacagcagagcaacggaacgctaaaagccaagaattagccgagtatagccaaaaaataaaagcatacgaacagaaaacgctagagccaatcaacgagaagattgccaaatatcaagctgacaaccctgaaataaaaatgcggagcttaggatttgtgaaaaaaattaaggctcaaggggcatataaagcggctcaagagcgaatggagcgagaaaaacagcaccaacaggaaaaacaacagagacatttagagcgagagagtggtttgagcttgtagctaacgccctacgcctacggcttcggttgttcaacccttaaagaactcgcaacaagttgcaaattctttaagggttcgcaataaaaacaaccgctaaacatttctgcccagcggttgaaaatttacctattcaccattacaatgatcaagcaggaaatttttttgattgccgtaaatgtccgtatatctagttgaggcacaacccgccaaagtcattgccccaaccagaacggcgaagctaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttcctcagcgcatgcatccgggctctgcgcttgaattgtgttgtagaaacacaacgtttttgaaaaaataagctattgttttatatcaaaatataatcatttttaaaataaaggttgcggcatttatcagatatttgttctgaaatgaagacgtatcgggtgtttgcccgatgtttttaggtttttatcaaatttacaaaaggaagcccatatgaaaaccttcttcaaaaccctttccgccgccgcactcgcgctcatcctcgcagcctgcggcggtcaaaaagacagcgcgcccgcagcctctgccgccgccccttctgccgataacggcgcggcgaaaaaagaaatcgtcttcggcacgaccgtcggcgacttcggcgatatggtcaaagaacaaatccaagccgagctggagaaaaaaggctacaccgtcaaattggtcgaatttaccgactatgtgcgcccgaatctggcattggcggagggcgagttggacatcaacgtcttccaacacaaaccctatcttgacgatttcaaaaaagaacacaacctggacatcaccgaagccttccaagtgccgaccgcgcctttgggactgtatccgggcaaactgaaatcgctggaagaagtcaaagacggcagcaccgtatccgcgcccaacgacccgtccaacttcgcacgcgccttggtgatgctgaacgaactgggttggatcaaactcaaagacggcatcaatccgctgaccgcatccaaagccgacatcgcggaaaacctgaaaaacatcaaaatcgtcgagcttgaagccgcacaactgccgcgcagccgcgccgacgtggattttgccgtcgtcaacggcaactacgccataagcagcggcatgaagctgaccgaagccctgttccaagagccgagctttgcctatgtcgcctggtctgccgtcaaaaccgccgacaaagacagccaatggcttaaagacgtaaccgaggcctataactccgacgcgttcaaagcctacgcgcacaaacgcttcgagggctacaaataccctgccgcatggaatgaaggcgcagccaaataagtgcggtcatcttcggtttccgtgtttcgtaaagtctggaaacgcggaagtcccctacgtgctgctgaagttgccccaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaaca

SEQ ID NO: 35 - DNA, 6821 bp, synthetic construct
  misc_feature 1..6821: note = pFP12-GNA1220 plasmid
  source 1..6821: mol_type = other DNA
ctgcaggccgtctgaaccgctctaaccgctttttctcggcttaatttttctgtctctgttataaaattgctattcatcttgttcttcttcaaaaaaaagttaagtaaaatacctacctaaatttttactagttcgcaatctacgagcttataacctcgttttttcaattcatttaaaaaatcagattttgagcctaattttgatctattgctatcgttacccgctagaaatacccagtaattacgcaaatcttcattggtaactttcgtaatatcggtgtaatgatcttcgagtatttttaagcaatctctagcccataaaccgtactcgtgattgctcatcttagggttttgcttatcgagtttgacgaacttcccatacttgtttttatgtggaaatactggccgttttgcaacttcttcaattttttgagctgttcgttttttactaccaatcacaaaatttaaagagtgaatagtacgcccacgcttgatttgttcaacctcaacgactaaatcagatttctcgttaatctcagttattgcaggttccaaaacacgttgatttaatgaattaaatctagggtatttattttcaacctgaagccattcttttagtttttctactgtaatttcacgactaccaacagagcgatattgtgtaattagctcataaattcgaattgaatgtacactgttgaaataagcgatatgtttgagttgatattgcgtgaattgccctttaagttgcgttaggtatggcataacttcatcagtcattgcaattctaaaacgcccctctttcttgaaatatgttctagaggaaacccaacgaaattcagttacacggtctttatcttcagttttaacacttcggtcataaatccgttttatagccgcctgaatttgcttataggcgttatcttggcttatttctggaaactcacggacaaaatcagccaccgtaaaatcaaaaatcttttgattagatttcggatccatagtcccaatagttaaagctaaaattctgatttcatcaatactcaatcggtaattggcttcaataaggctattagcctttacaacaactaaatcatttggcataagacaacaaatttcctgtttaaaacaacaagcaaaatatacctgttgtttatatataaaacaacaagtattttcttaaaagttgtctataacaggaaatttgttgtcttataacaggaaatttgttgtcgtataacaggaaatttgttgtcgtataacaggaaatttgttgtcgtataagtttgtaacttattgattttactggttttaaaaacgccgaaaacaagtaaaaaacaaaaatataaaaatatagggactttcgtcccttttttgggctttcagccctaattttttctttttttcaggatttaaaattacaaaacccttacagagcaagtaaacttgtttgcttgttctgcaagggttcagcaaccgtagccgtcaggcgtagggcggtagcctataaaagccatttaattttatctttaaacttccttttaaatgctttgagtgggtgtcttttatcgtactcatcaatccttttttgcattctttcgtttgctttgtgatcggcaaattttgaataagatttttccatctcatctaacattctatcaatccgttttttatgttgccatttcaggtaaacataaacacttatagcaattaaagacaatatcaatacattgtaaaaaatgattgttacaatttcgctcacagttattttttacctttttcaatttcttcattgataaatgcactcaattcatcaaatttcttgtcatcattgataaatttacgcaacttagggaagtttctatctacatctaaaagagggttatttattatttcatttagccaaaaagcccctaataaaaccttgtaatgcgtagctttcttacgcttttctgcttgttcttttgacttaatcgcacgaattttcgctttgatttcgtcctgcttgcgttgtaaatctgcttgttgctgttccaatcttgtaagtttttcgcttgccatactagcccctttatatagttagaaattatcgttattttattcagtaggtgctaggcttgcaagtgttctgttcattacgttaaaataacgtaatgcccacttatcagtttctcttcgagaaactggtgggcaagcgtaccgcttgaccgtttcgcaatactcaacactatggcaatctatcatttaaacgttcgctattgcagtaaaagcaaagggcaatcagctcaagccaaaaacgactacatcaaccgcaatgataaatattcaaagcggttagatgatttacagttttcaggctatggtaatatgccaaaatttgccgaagataatccgcaagaattttggcgattgtcagatatttacgagcgagctaatgcccgagtttgtactgaaattgaatttgctttacctagagaattaaccctagaacaacagcaaaaattagtaagttcgtttatagaaaatacggttgatagcggtagcaataaactaccctactctttcgctatccataccgataaaaataatcataatccccattgtcatttgatattttcagaacgccaacttgacggcatagaccgtacagccgagcagttttttaaacgtgctaatactaaatccccagaaaagggcggagcgatgaaaacggcagattttcgagatcgtgagtttatccaatctgtccgaaaaacgtggagagagcaagctaatcaagccttagagcaatacggatatgccgcacgaattgacgaacgtagctacaaggaacaaggcatagagcaagccccaagagcaagaattgacagggtaacgtggcaagaattgaaccgattagagcaagaagaacgccaaatcgtgcaagagcttgcacttaaaggacaagaaattaacaaagaaaaatcctacttgcagaaaatcgaagaaaaacaggctcaaggaatgggcaaatatgaatccaaattcgcagctgcgttttctaaattatcggaaagtgccctaaaacacgatttaagcaacgaaaaagaaaaagacagtaaaatacacactcaagaagaaaaagtgcctcaaaatcgcattcaggggctttctcaagcagattttgatcagtttttaattgatgaatggctacctcaaatagaaaaatacgttaaagcccaagaaaagcgggacggaatggaagtagagatcacgcaatacgacaaggatttacagcgtattcagggagactataacaagctcacagataaaaatcagggttttctcggtttatgggaaactaaagagcaaaaagcaaagaaaaaagagcttgaagatgaatacaaacatacagcagagcaacggaacgctaaaagccaagaattagccgagtatagccaaaaaataaaagcatacgaacagaaaacgctagagccaatcaacgagaagattgccaaatatcaagctgacaaccctgaaataaaaatgcggagcttaggatttgtgaaaaaaattaaggctcaaggggcatataaagcggctcaagagcgaatggagcgagaaaaacagcaccaacaggaaaaacaacagagacatttagagcgagagagtggtttgagcttgtagctaacgccctacgcctacggcttcggttgttcaacccttaaagaactcgcaacaagttgcaaattctttaagggttcgcaataaaaacaaccgctaaacatttctgcccagcggttgaaaatttacctattcaccattacaatgatcaagcaggaaatttttttgattgccgtaaatgtccgtatatctagttgaggcacaacccgccaaagtcattgccccaaccagaacggcgaagctaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttcctcagcgcatgcatccgggctctgcgcttgaattgtgttgtagaaacacaacgtttttgaaaaaataagctattgttttatatcaaaatataatcatttttaaaataaaggttgcggcatttatcagatatttgttctgaaatgaagacgtatcgggtgtttgcccgatgtttttaggtttttatcaaatttacaaaaggaagcccatatggaatttttcattatcttgttggcagccgtcgccgttttcggcttcaaatcctttgtcgtcatcccccagcaggaagtccacgttgtcgaaaggctcgggcgtttccatcgcgccctgacggccggtttgaatattttgattccctttatcgaccgcgtcgcctaccgccattcgctgaaagaaatccctttagacgtacccagccaggtctgcatcacgcgcgataatacgcaattgactgttgacggcatcatctatttccaagtaaccgatcccaaactcgcctcatacggttcgagcaactacattatggcaattacccagcttgcccaaacgacgctgcgttccgttatcgggcgtatggagttggacaaaacgtttgaagaacgcgacgaaatcaacagtaccgtcgtctccgccctcgatgaagccgccggggcttggggtgtgaaagtcctccgttacgaaatcaaggatttggttccgccgcaagaaatccttcgcgcaatgcaggcacaaattaccgccgaacgcgaaaaacgcgcccgtattgccgaatccgaaggccgtaaaatcgaacaaatcaaccttgccagtggtcagcgtgaagccgaaatccaacaatccgaaggcgaggctcaggctgcggtcaatgcgtccaatgccgagaaaatcgcccgcatcaaccgcgccaaaggcgaagcggaatccctgcgccttgttgccgaagccaatgccgaagccatccgtcaaattgccgccgcccttcaaacccaaggcggggcggatgcggtcaatctgaagattgcggaacaatacgtagccgcgttcaacaatcttgccaaagaaagcaatacgctgattatgcccgccaatgttgccgacatcggcagcctgatttctgccggcatgaaaattatcgacagcagcaaaaccgccaaataagtgcggtcatcttcggtttccgtgtttcgtaaagtctggaaacgcggaagtcccctacgtgctgctgaagttgccccaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaaca

SEQ ID NO: 36 - DNA, 6374 bp, synthetic construct
  misc_feature 1..6374: note = pFP12-GNA1220_helix-bab plasmid
  source 1..6374: mol_type = other DNA
ctgcaggccgtctgaaccgctctaaccgctttttctcggcttaatttttctgtctctgttataaaattgctattcatcttgttcttcttcaaaaaaaagttaagtaaaatacctacctaaatttttactagttcgcaatctacgagcttataacctcgttttttcaattcatttaaaaaatcagattttgagcctaattttgatctattgctatcgttacccgctagaaatacccagtaattacgcaaatcttcattggtaactttcgtaatatcggtgtaatgatcttcgagtatttttaagcaatctctagcccataaaccgtactcgtgattgctcatcttagggttttgcttatcgagtttgacgaacttcccatacttgtttttatgtggaaatactggccgttttgcaacttcttcaattttttgagctgttcgttttttactaccaatcacaaaatttaaagagtgaatagtacgcccacgcttgatttgttcaacctcaacgactaaatcagatttctcgttaatctcagttattgcaggttccaaaacacgttgatttaatgaattaaatctagggtatttattttcaacctgaagccattcttttagtttttctactgtaatttcacgactaccaacagagcgatattgtgtaattagctcataaattcgaattgaatgtacactgttgaaataagcgatatgtttgagttgatattgcgtgaattgccctttaagttgcgttaggtatggcataacttcatcagtcattgcaattctaaaacgcccctctttcttgaaatatgttctagaggaaacccaacgaaattcagttacacggtctttatcttcagttttaacacttcggtcataaatccgttttatagccgcctgaatttgcttataggcgttatcttggcttatttctggaaactcacggacaaaatcagccaccgtaaaatcaaaaatcttttgattagatttcggatccatagtcccaatagttaaagctaaaattctgatttcatcaatactcaatcggtaattggcttcaataaggctattagcctttacaacaactaaatcatttggcataagacaacaaatttcctgtttaaaacaacaagcaaaatatacctgttgtttatatataaaacaacaagtattttcttaaaagttgtctataacaggaaatttgttgtcttataacaggaaatttgttgtcgtataacaggaaatttgttgtcgtataacaggaaatttgttgtcgtataagtttgtaacttattgattttactggttttaaaaacgccgaaaacaagtaaaaaacaaaaatataaaaatatagggactttcgtcccttttttgggctttcagccctaattttttctttttttcaggatttaaaattacaaaacccttacagagcaagtaaacttgtttgcttgttctgcaagggttcagcaaccgtagccgtcaggcgtagggcggtagcctataaaagccatttaattttatctttaaacttccttttaaatgctttgagtgggtgtcttttatcgtactcatcaatccttttttgcattctttcgtttgctttgtgatcggcaaattttgaataagatttttccatctcatctaacattctatcaatccgttttttatgttgccatttcaggtaaacataaacacttatagcaattaaagacaatatcaatacattgtaaaaaatgattgttacaatttcgctcacagttattttttacctttttcaatttcttcattgataaatgcactcaattcatcaaatttcttgtcatcattgataaatttacgcaacttagggaagtttctatctacatctaaaagagggttatttattatttcatttagccaaaaagcccctaataaaaccttgtaatgcgtagctttcttacgcttttctgcttgttcttttgacttaatcgcacgaattttcgctttgatttcgtcctgcttgcgttgtaaatctgcttgttgctgttccaatcttgtaagtttttcgcttgccatactagcccctttatatagttagaaattatcgttattttattcagtaggtgctaggcttgcaagtgttctgttcattacgttaaaataacgtaatgcccacttatcagtttctcttcgagaaactggtgggcaagcgtaccgcttgaccgtttcgcaatactcaacactatggcaatctatcatttaaacgttcgctattgcagtaaaagcaaagggcaatcagctcaagccaaaaacgactacatcaaccgcaatgataaatattcaaagcggttagatgatttacagttttcaggctatggtaatatgccaaaatttgccgaagataatccgcaagaattttggcgattgtcagatatttacgagcgagctaatgcccgagtttgtactgaaattgaatttgctttacctagagaattaaccctagaacaacagcaaaaattagtaagttcgtttatagaaaatacggttgatagcggtagcaataaactaccctactctttcgctatccataccgataaaaataatcataatccccattgtcatttgatattttcagaacgccaacttgacggcatagaccgtacagccgagcagttttttaaacgtgctaatactaaatccccagaaaagggcggagcgatgaaaacggcagattttcgagatcgtgagtttatccaatctgtccgaaaaacgtggagagagcaagctaatcaagccttagagcaatacggatatgccgcacgaattgacgaacgtagctacaaggaacaaggcatagagcaagccccaagagcaagaattgacagggtaacgtggcaagaattgaaccgattagagcaagaagaacgccaaatcgtgcaagagcttgcacttaaaggacaagaaattaacaaagaaaaatcctacttgcagaaaatcgaagaaaaacaggctcaaggaatgggcaaatatgaatccaaattcgcagctgcgttttctaaattatcggaaagtgccctaaaacacgatttaagcaacgaaaaagaaaaagacagtaaaatacacactcaagaagaaaaagtgcctcaaaatcgcattcaggggctttctcaagcagattttgatcagtttttaattgatgaatggctacctcaaatagaaaaatacgttaaagcccaagaaaagcgggacggaatggaagtagagatcacgcaatacgacaaggatttacagcgtattcagggagactataacaagctcacagataaaaatcagggttttctcggtttatgggaaactaaagagcaaaaagcaaagaaaaaagagcttgaagatgaatacaaacatacagcagagcaacggaacgctaaaagccaagaattagccgagtatagccaaaaaataaaagcatacgaacagaaaacgctagagccaatcaacgagaagattgccaaatatcaagctgacaaccctgaaataaaaatgcggagcttaggatttgtgaaaaaaattaaggctcaaggggcatataaagcggctcaagagcgaatggagcgagaaaaacagcaccaacaggaaaaacaacagagacatttagagcgagagagtggtttgagcttgtagctaacgccctacgcctacggcttcggttgttcaacccttaaagaactcgcaacaagttgcaaattctttaagggttcgcaataaaaacaaccgctaaacatttctgcccagcggttgaaaatttacctattcaccattacaatgatcaagcaggaaatttttttgattgccgtaaatgtccgtatatctagttgaggcacaacccgccaaagtcattgccccaaccagaacggcgaagctaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttcctcagcgcatgcatccgggctctgcgcttgaattgtgttgtagaaacacaacgtttttgaaaaaataagctattgttttatatcaaaatataatcatttttaaaataaaggttgcggcatttatcagatatttgttctgaaatgaagacgtatcgggtgtttgcccgatgtttttaggtttttatcaaatttacaaaaggaagcccatatggtgaaccgaactgccttctgcctttctctgaccgccgccctgattctgaccgcctgcagcagcggagggggtggtgaacaaatcaaccttgccagtggtcagcgtgaagccgaaatccaacaatccgaaggcgaggctcaggctgcggtcaatgcgtccaatgccgagaaaatcgcccgcatcaaccgcgccaaaggcgaagcggaatccctgcgccttgttgccgaagccaatgccgaagccatccgtcaaattgccgccgcccttcaaacccaaggcggggcggatgcggtcaatctgaagattgcggaacaatacgtagccgcgttcaacaatcttgccaaagaaagcaatacgctgattatgcccgccaatgttgccgacatcggcagcctgatttctgccggcatgaaaattatcgacagcagcaaaaccgccaaataaaggcctactagtcggccgtacgggccctttcgtctcgcgcgtttcggtgattaagtgcggtcatcttcggtttccgtgtttcgtaaagtctggaaacgcggaagtcccctacgtgctgctgaagttgccccaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaaca

SEQ ID NO: 37 - protein, 426 aa, synthetic construct
  REGION 1..426: note = Neisseria gonorrhoeae NHBA protein
MFKRSVIAMACIFPLSACGGGGGGSPDVKSADTPSKPAAPVVAENAGEGVLPKEKKDEEAAGGAPQADTQDATAGEGSQDMAAVSAENTGNGGAATTDNPKNEDAGAQNDMPQNAAESANQTGNNQPAGSSDSAPASNPAPANGGSDFGRTNVGNSVVIDGPSQNITLTHCKGDPCNGDNLLDEEAPPKSEFESLSDEEKIKKYKKDGEKFTGLVAIKVENNGLNKYTIIYQAQPTRSARSRRSLPAEIPLIPVNQADTLIVDGEAVSLTGHSGNIFAPEGNYRYLTYGAEKLPGGSYALRVQGEPAKGEMLAGTAVYNGEVLHFHMENGRPYPSGGRFAAKVDFGSKSVDGIIDSSDDLHMGTQKFKAAIDGNGFKGTWTENGGGDVSGRFYGPAGEEVAGKYSYRPTDAEKGGFGVFAGKKDRD

SEQ ID NO: 38 - DNA, 1281 bp, synthetic construct
  misc_feature 1..1281: note = pFP12-NHBA plasmid
  source 1..1281: mol_type = other DNA
atgtttaaacgcagtgtgattgcaatggcttgtatttttcccctttcagcctgtgggggcggcggtggcggatcgcccgatgtcaagtcggcggacacgccgtcaaaaccggccgcccccgttgttgctgaaaatgccggggaaggggtgctgccgaaagaaaagaaagatgaggaggcagcgggcggtgcgccgcaagccgatacgcaggacgcaaccgccggagaaggcagccaagatatggcggcagtttcggcagaaaatacaggcaatggcggtgcggcaacaacggacaaccccaaaaatgaagacgcgggggcgcaaaatgatatgccgcaaaatgccgccgaatccgcaaatcaaacagggaacaaccaacccgccggttcttcagattccgcccccgcgtcaaaccctgcccctgcgaatggcggtagcgattttggaaggacgaacgtgggcaattctgttgtgattgacggaccgtcgcaaaatataacgttgacccactgtaaaggcgatccttgtaatggtgataatttattggatgaagaagcaccgccaaaatcagaatttgaatcgttaagtgatgaagagaaaattaagaaatataaaaaagatggagaaaagtttactggtttggttgctataaaagttgaaaataatgggttgaataaatataccattatttatcaagctcagcctactcgttctgcacggtcgaggaggtcgcttccggccgagattccgctgattcccgtcaatcaggccgatacgctgattgtggatggggaagcggtcagcctgacggggcattccggcaatatcttcgcgcccgaagggaattaccggtatctgacttacggggcggaaaaattgcccggcggatcgtatgccctccgtgtgcaaggcgaaccggcaaaaggcgaaatgcttgcgggcacggccgtgtacaacggcgaagtgctgcatttccacatggaaaacggccgtccgtacccgtccggaggcaggtttgccgcaaaagtcgatttcggcagcaaatctgtggacggcattatcgacagcagcgatgatttgcacatgggtacgcaaaaattcaaagccgccatcgatggaaacggctttaaggggacttggacggaaaatggcggcggggatgtttccggaaggttttacggcccggccggcgaggaagtggcgggaaaatacagctatcgcccgacagatgctgaaaagggcggattcggcgtgtttgccggcaaaaaagatcgggattga

SEQ ID NO: 39 - DNA, 8066 bp, synthetic construct
  misc_feature 1..8066: note = pFP12-NHBA plasmid
  source 1..8066: mol_type = other DNA
ctgcaggccgtctgaaccgctctaaccgctttttctcggcttaatttttctgtctctgttataaaattgctattcatcttgttcttcttcaaaaaaaagttaagtaaaatacctacctaaatttttactagttcgcaatctacgagcttataacctcgttttttcaattcatttaaaaaatcagattttgagcctaattttgatctattgctatcgttacccgctagaaatacccagtaattacgcaaatcttcattggtaactttcgtaatatcggtgtaatgatcttcgagtatttttaagcaatctctagcccataaaccgtactcgtgattgctcatcttagggttttgcttatcgagtttgacgaacttcccatacttgtttttatgtggaaatactggccgttttgcaacttcttcaattttttgagctgttcgttttttactaccaatcacaaaatttaaagagtgaatagtacgcccacgcttgatttgttcaacctcaacgactaaatcagatttctcgttaatctcagttattgcaggttccaaaacacgttgatttaatgaattaaatctagggtatttattttcaacctgaagccattcttttagtttttctactgtaatttcacgactaccaacagagcgatattgtgtaattagctcataaattcgaattgaatgtacactgttgaaataagcgatatgtttgagttgatattgcgtgaattgccctttaagttgcgttaggtatggcataacttcatcagtcattgcaattctaaaacgcccctctttcttgaaatatgttctagaggaaacccaacgaaattcagttacacggtctttatcttcagttttaacacttcggtcgtaaatccgttttatagccgcctgaatttgcttataggcgttatcttggcttatttctggaaactcacggacaaaatcagccaccgtaaaatcaaaaatcttttgattagatttcggatccatagtcccgatagttaaagctaaaattctgatttcatcaatactcaatcggtaattggcttcaataaggctattagcctttacaacaactaaatcatttggcataagacaacaaatttcctgtttaaaacaacaagcaaaatatacctgttgtttatatataaaacaacaagtattttcttaaaagttgtctataacaggaaatttgttgtcgtataacaggaaatttgttgtcttataacaggaaatttgttgtcgtataacaggaaatttgttgtcgtataagtttgtaacttattgattttactggttttaaaaacgccgaaaacaagtaaaaaacaaaaatataaaaatatagggactttcgtcccttttttgggctttcagccctaattttttcttttttcaggattaaaaattacaaaacccttacagagcaagtaaacttgtttgcttgttctgcaagggttcagcaaccgtagccgtcaggcgtagggcggtagcctataaaagccatttaattttatctttaaacttccttttaaatgctttgagtgggtgtcttttatcgtactcatcaatccttttttgcattctttcgtttgctttgtgatcggcaaattttgaataagatttttccatctcatctaacattctatcaatccgttttttatgttgccatttcaggtaaacataaacacttatagcaattaaagacaatatcaatacattgtaaaaaatgattgttacaatttcgctcacagttattttttacctttttcaatttcttcattgataaatgcactcaattcatcaaatttcttgtcatcattgataaatttacgcaacttagggaagtttctatctacatctaaaagagggctatttattacttcatttagccaaaaagcccctaataaaaccttgtaatgcgtagctttcttacgcttttctgcttgttcttttgacttaatcgcacgaattttcgctttgatttcgtcctgcttgcgttgtaaatctgcttgttgctgttccaatcttgtaagtttttcgcttgccatactagcccctttatatagttagaaattatcgttattttattcagtaggtgctaggcttgcaagtgttctgttcattacgttaaaataacgtaatgcccacttatcagtttctcttcgagaaactggtgggcaagcgtaccgcttgaccgtttcgcaatactcaacactatggcaatctatcatttaaacgttcgctattgcagtaaaagcaaagggcaatcagctcaagccaaaaacgactacatcaaccgcaatgataaatattcaaagcggttagatgatttacagttttcaggctatggtaatatgccaaaatttgccgaagataatccgcaagaattttggcgattgtcagatatttacgagcgagctaatgcccgagtttgtactgaaattgaatttgctttacctagagaattaaccctagaacaacagcaaaaattagtaagttcgtttatagaaaatacggttgatagcggtagcaataaactaccctactctttcgctatccataccgataaaaataatcataatccccattgtcatttgatattttcagaacgccaacttgacggcatagaccgtacagccgagcagttttttaaacgtgctaatactaaatccccagaaaagggcggagcgatgaaaacggcagattttcgagatcgtgagtttatccaatctgtccgaaaaacgtggagagagcaagctaatcaagccttagagcaatacggatatgccgcacgaattgacgaacgtagctacaaggaacaaggcatagagcaagccccaagagcaagaattgacagggtaacgtggcaagaattgaaccgattagagcaagaagaacgccaaatcgtgcaagagcttgcacttaaaggacaagaaattaacaaagaaaaatcctacttgcagaaaatcgaagaaaaacaggctcaaggaatgggcaaatatgaatccaaattcgcagctgcgttttctaaattatcggaaagtgccctaaaacacgatttaagcaacgaaaaagaaaaagacagtaaaatacacactcaagaagaaaaagtgcctcaaaatcgcattcaggggctttctcaagcagattttgatcagtttttaattgatgaatggctacctcaaatagaaaaatacgttaaagcccaagaaaagcgggacggaatggaagtagagatcacgcaatacgacaaggatttacagcgtattcagggagactataacaagctcacagataaaaatcagggttttctcggtttatgggaaactaaagagcaaaaagcaaagaaaaaagagcttgaagatgaatacaaacatacagcagagcaacggaacgctaaaagccaagaattagccgagtatagccaaaaaataaaagcatacgaacagaaaacgctagagccaatcaacgagaagattgccaaatatcaagctgacaaccctgaaataaaaatgcggagcttaggatttgtgaaaaaaattaaggctcgaggggcatataaagcggctcaagagcgaatggagcgagaaaaacagcaccaacaggaaaaacaacagagacatttagagcgagagagtggtttgagcttgtagctaacgccctacgcctacggcttcggttgttcaacccttaaagaactcgcaacaagttgcaaattctttaagggttcgcaataaaaacaaccgctaaacatttctgcccagcggttgaaaatttacctattcaccattacaatgatcaagcaggaaatttttttgattgccgtaaatgtccgtatatctagttgaggcacaacccgccaaagtcattgccccaaccagaacggcgaaagctaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacgggaaacagctatgaccatgattacgccaagcttcctcagcgcatgcatccgggcagcagcgcatcggctcgcacgaggtctgcgcttgaattgtgttgtagaaacacaacgtttttgaaaaaataagctattgttttatatcaaaatataatcatttttaaaataaaggttgcggcatttatcagatatttgttctgaaatgaagacgtatcgggtgtttgcccgatgtttttaggtttttatcaaatttacaaaaggaagcccatatgtttaaacgcagtgtgattgcaatggcttgtatttttcccctttcagcctgtgggggcggcggtggcggatcgcccgatgtcaagtcggcggacacgccgtcaaaaccggccgcccccgttgttgctgaaaatgccggggaaggggtgctgccgaaagaaaagaaagatgaggaggcagcgggcggtgcgccgcaagccgatacgcaggacgcaaccgccggagaaggcagccaagatatggcggcagtttcggcagaaaatacaggcaatggcggtgcggcaacaacggacaaccccaaaaatgaagacgcgggggcgcaaaatgatatgccgcaaaatgccgccgaatccgcaaatcaaacagggaacaaccaacccgccggttcttcagattccgcccccgcgtcaaaccctgcccctgcgaatggcggtagcgattttggaaggacgaacgtgggcaattctgttgtgattgacggaccgtcgcaaaatataacgttgacccactgtaaaggcgatccttgtaatggtgataatttattggatgaagaagcaccgccaaaatcagaatttgaatcgttaagtgatgaagagaaaattaagaaatataaaaaagatggagaaaagtttactggtttggttgctataaaagttgaaaataatgggttgaataaatataccattatttatcaagctcagcctactcgttctgcacggtcgaggaggtcgcttccggccgagattccgctgattcccgtcaatcaggccgatacgctgattgtggatggggaagcggtcagcctgacggggcattccggcaatatcttcgcgcccgaagggaattaccggtatctgacttacggggcggaaaaattgcccggcggatcgtatgccctccgtgtgcaaggcgaaccggcaaaaggcgaaatgcttgcgggcacggccgtgtacaacggcgaagtgctgcatttccacatggaaaacggccgtccgtacccgtccggaggcaggtttgccgcaaaagtcgatttcggcagcaaatctgtggacggcattatcgacagcagcgatgatttgcacatgggtacgcaaaaattcaaagccgccatcgatggaaacggctttaaggggacttggacggaaaatggcggcggggatgtttccggaaggttttacggcccggccggcgaggaagtggcgggaaaatacagctatcgcccgacagatgctgaaaagggcggattcggcgtgtttgccggcaaaaaagatcgggattgaaggcctcctgcaggactagtcggccgtacctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtgggccctttcgtctcgcgcgtttcggtgatagcttaagtgcggtcatcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaaca

SEQ ID NO: 40 - DNA, 6266 bp, synthetic construct
  misc_feature 1..6266: note = pBS-FHbpKO-MetQ plasmid
  source 1..6266: mol_type = other DNA
gcatgcatccgggcagcagcgcatcggctcgcacgaggtctgcgcttgaattgtgttgtagaaacacaacgtttttgaaaaaataagctattgttttatatcaaaatataatcatttttaaaataaaggttgcggcatttatcagatatttgttctgaaatgaagacgtatcgggtgtttgcccgatgtttttaggtttttatcaaatttacaaaaggaagcccatatgaaaaccttcttcaaaaccctttccgccgccgcactcgcgctcatcctcgcagcctgcggcggtcaaaaagacagcgcgcccgcagcctctgccgccgccccttctgccgataacggcgcggcgaaaaaagaaatcgtcttcggcacgaccgtcggcgacttcggcgatatggtcaaagaacaaatccaagccgagctggagaaaaaaggctacaccgtcaaattggtcgaatttaccgactatgtgcgcccgaatctggcattggcggagggcgagttggacatcaacgtcttccaacacaaaccctatcttgacgatttcaaaaaagaacacaacctggacatcaccgaagccttccaagtgccgaccgcgcctttgggactgtatccgggcaaactgaaatcgctggaagaagtcaaagacggcagcaccgtatccgcgcccaacgacccgtccaacttcgcacgcgccttggtgatgctgaacgaactgggttggatcaaactcaaagacggcatcaatccgctgaccgcatccaaagccgacatcgcggaaaacctgaaaaacatcaaaatcgtcgagcttgaagccgcacaactgccgcgcagccgcgccgacgtggattttgccgtcgtcaacggcaactacgccataagcagcggcatgaagctgaccgaagccctgttccaagagccgagctttgcctatgtcaactggtctgccgtcaaaaccgccgacaaagacagccaatggcttaaagacgtaaccgaggcctataactccgacgcgttcaaagcctacgcgcacaaacgcttcgagggctacaaataccctgccgcatggaatgaaggcgcagccaaataaggcagtcgtataacctgcaggccaagcaaaaccattgtgaaaatgccgtccgaacacgataatttaccgttcggacggcattttgtattgcaccgtccgacggcatgcccaaggggggaaatccctattttcaggccaaccgctatataatgccgtctgaaccaacgagagaatgccatgcaagctgattttaaccgtcccgtcctggccgtcgataccggtacttcccgtttgtcgctcgcgctgcgtgccgacggcgaaacccgtctgttccatcaggaagtcggcagccgccagtccgaactgattctgccggaaatccgcaccctattccgcgatgcaggcattaccgccgccgatttgggtgcggtcgtgtacgcacagggtcccggcgcgtttaccggactgcgtatcggcatcggtgtagctcagggtttggcaacgccgtttgatacccccttaatcggcgtaccctcgctcgatgccgccgcctcgctgccgccgccgcaaagctgcatccttgccgctacggacgctcgtatgggcgaagtgttttatgcatggttcgatacgctgctcgagggggggcccggtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctccaccgcggtggcggccgctctagaccagccacggcgcatacaaattcacccgtccgcccacaggcgatgtattacgtatcgaccgcatcaaagaaatccaccaagccctgcccaatacacacatcgtgatgcacggctccagctccgttccgcaagaatggctgaaagtcatcaacgaatacggcggcaatatcggcgaaacctacggcgtgccggttgaagaaatcgtcgaaggcatcaaacacggcgtgcgcaaagtcaacatcgataccgacttgcgccttgcttctaccggcgcggtacgccgctaccttgccgaaaatccgtccgactttgacccgcgcaaatacctgagcaaaaccattgaggccatgaagcaaatctgcctcgaccgttatcttgcgtttggctgcgaaggtcaggcaggcaaaatcaaacctgtttcgttggaaaaaatggcaagccgttatgccaagggcgaattgaaccaaatcgtcaaataacaggttgcctgtaaacaaaatgccgtcccatgggcaaacttaagagtgtgttgatagtgcagtatcttaaaattttgtgtataataggaattgaagttaaattagatgctaaaaatttgtaattaagaaggagggattcgtcatgttggtattccaaatgcgtaatgtagataaaacatctactgttttgaaacagactaaaaacagtgattacgcagataaataaatacgttagattaattcctaccagtgactaatcttatgactttttaaacagataactaaaattacaaacaaatcgtttaacttctgtatttatttacagatgtaatcacttcaggagtaattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaacattcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccattacttatgagcaagtattgtctatttttaatagttatctattatttaacgggaggaaataattctatgagtcgcttttttaaatttggaaagttacacgttactaaagggaatggagataaattattagatatactactgacagcttccaagaagctaaagaggtccctttcgacggccccgggcctcagc

SEQ ID NO: 41 - DNA, 6266 bp, synthetic construct
  misc_feature 1..6266: note = pBS-FHbpKO-MetQSM plasmid
  source 1..6266: mol_type = other DNA
gcatgcatccgggcagcagcgcatcggctcgcacgaggtctgcgcttgaattgtgttgtagaaacacaacgtttttgaaaaaataagctattgttttatatcaaaatataatcatttttaaaataaaggttgcggcatttatcagatatttgttctgaaatgaagacgtatcgggtgtttgcccgatgtttttaggtttttatcaaatttacaaaaggaagcccatatgaaaaccttcttcaaaaccctttccgccgccgcactcgcgctcatcctcgcagcctgcggcggtcaaaaagacagcgcgcccgcagcctctgccgccgccccttctgccgataacggcgcggcgaaaaaagaaatcgtcttcggcacgaccgtcggcgacttcggcgatatggtcaaagaacaaatccaagccgagctggagaaaaaaggctacaccgtcaaattggtcgaatttaccgactatgtgcgcccgaatctggcattggcggagggcgagttggacatcaacgtcttccaacacaaaccctatcttgacgatttcaaaaaagaacacaacctggacatcaccgaagccttccaagtgccgaccgcgcctttgggactgtatccgggcaaactgaaatcgctggaagaagtcaaagacggcagcaccgtatccgcgcccaacgacccgtccaacttcgcacgcgccttggtgatgctgaacgaactgggttggatcaaactcaaagacggcatcaatccgctgaccgcatccaaagccgacatcgcggaaaacctgaaaaacatcaaaatcgtcgagcttgaagccgcacaactgccgcgcagccgcgccgacgtggattttgccgtcgtcaacggcaactacgccataagcagcggcatgaagctgaccgaagccctgttccaagagccgagctttgcctatgtcgcctggtctgccgtcaaaaccgccgacaaagacagccaatggcttaaagacgtaaccgaggcctataactccgacgcgttcaaagcctacgcgcacaaacgcttcgagggctacaaataccctgccgcatggaatgaaggcgcagccaaataaggcagtcgtataacctgcaggccaagcaaaaccattgtgaaaatgccgtccgaacacgataatttaccgttcggacggcattttgtattgcaccgtccgacggcatgcccaaggggggaaatccctattttcaggccaaccgctatataatgccgtctgaaccaacgagagaatgccatgcaagctgattttaaccgtcccgtcctggccgtcgataccggtacttcccgtttgtcgctcgcgctgcgtgccgacggcgaaacccgtctgttccatcaggaagtcggcagccgccagtccgaactgattctgccggaaatccgcaccctattccgcgatgcaggcattaccgccgccgatttgggtgcggtcgtgtacgcacagggtcccggcgcgtttaccggactgcgtatcggcatcggtgtagctcagggtttggcaacgccgtttgatacccccttaatcggcgtaccctcgctcgatgccgccgcctcgctgccgccgccgcaaagctgcatccttgccgctacggacgctcgtatgggcgaagtgttttatgcatggttcgatacgctgctcgagggggggcccggtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctccaccgcggtggcggccgctctagaccagccacggcgcatacaaattcacccgtccgcccacaggcgatgtattacgtatcgaccgcatcaaagaaatccaccaagccctgcccaatacacacatcgtgatgcacggctccagctccgttccgcaagaatggctgaaagtcatcaacgaatacggcggcaatatcggcgaaacctacggcgtgccggttgaagaaatcgtcgaaggcatcaaacacggcgtgcgcaaagtcaacatcgataccgacttgcgccttgcttctaccggcgcggtacgccgctaccttgccgaaaatccgtccgactttgacccgcgcaaatacctgagcaaaaccattgaggccatgaagcaaatctgcctcgaccgttatcttgcgtttggctgcgaaggtcaggcaggcaaaatcaaacctgtttcgttggaaaaaatggcaagccgttatgccaagggcgaattgaaccaaatcgtcaaataacaggttgcctgtaaacaaaatgccgtcccatgggcaaacttaagagtgtgttgatagtgcagtatcttaaaattttgtgtataataggaattgaagttaaattagatgctaaaaatttgtaattaagaaggagggattcgtcatgttggtattccaaatgcgtaatgtagataaaacatctactgttttgaaacagactaaaaacagtgattacgcagataaataaatacgttagattaattcctaccagtgactaatcttatgactttttaaacagataactaaaattacaaacaaatcgtttaacttctgtatttatttacagatgtaatcacttcaggagtaattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaacattcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccattacttatgagcaagtattgtctatttttaatagttatctattatttaacgggaggaaataattctatgagtcgcttttttaaatttggaaagttacacgttactaaagggaatggagataaattattagatatactactgacagcttccaagaagctaaagaggtccctttcgacggccccgggcctcagc

SEQ ID NO: 42 - DNA, 6350 bp, synthetic construct
  misc_feature 1..6350: note = pBS-FHbpKO-GNA1220 plasmid
  source 1..6350: mol_type = other DNA
gcatgcatccgggcagcagcgcatcggctcgcacgaggtctgcgcttgaattgtgttgtagaaacacaacgtttttgaaaaaataagctattgttttatatcaaaatataatcatttttaaaataaaggttgcggcatttatcagatatttgttctgaaatgaagacgtatcgggtgtttgcccgatgtttttaggtttttatcaaatttacaaaaggaagcccatatgatggaatttttcattatcttgttggcagccgtcgccgttttcggcttcaaatcctttgtcgtcatcccccagcaggaagtccacgttgtcgaaaggctcgggcgtttccatcgcgccctgacggccggtttgaatattttgattccctttatcgaccgcgtcgcctaccgccattcgctgaaagaaatccctttagacgtacccagccaggtctgcatcacgcgcgataatacgcaattgactgttgacggcatcatctatttccaagtaaccgatcccaaactcgcctcatacggttcgagcaactacattatggcaattacccagcttgcccaaacgacgctgcgttccgttatcgggcgtatggagttggacaaaacgtttgaagaacgcgacgaaatcaacagtaccgtcgtctccgccctcgatgaagccgccggggcttggggtgtgaaagtcctccgttacgaaatcaaggatttggttccgccgcaagaaatccttcgcgcaatgcaggcacaaattaccgccgaacgcgaaaaacgcgcccgtattgccgaatccgaaggccgtaaaatcgaacaaatcaaccttgccagtggtcagcgtgaagccgaaatccaacaatccgaaggcgaggctcaggctgcggtcaatgcgtccaatgccgagaaaatcgcccgcatcaaccgcgccaaaggcgaagcggaatccctgcgccttgttgccgaagccaatgccgaagccatccgtcaaattgccgccgcccttcaaacccaaggcggggcggatgcggtcaatctgaagattgcggaacaatacgtagccgcgttcaacaatcttgccaaagaaagcaatacgctgattatgcccgccaatgttgccgacatcggcagcctgatttctgccggcatgaaaattatcgacagcagcaaaaccgccaaataaggcagtcgtataacctgcaggccaagcaaaaccattgtgaaaatgccgtccgaacacgataatttaccgttcggacggcattttgtattgcaccgtccgacggcatgcccaaggggggaaatccctattttcaggccaaccgctatataatgccgtctgaaccaacgagagaatgccatgcaagctgattttaaccgtcccgtcctggccgtcgataccggtacttcccgtttgtcgctcgcgctgcgtgccgacggcgaaacccgtctgttccatcaggaagtcggcagccgccagtccgaactgattctgccggaaatccgcaccctattccgcgatgcaggcattaccgccgccgatttgggtgcggtcgtgtacgcacagggtcccggcgcgtttaccggactgcgtatcggcatcggtgtagctcagggtttggcaacgccgtttgatacccccttaatcggcgtaccctcgctcgatgccgccgcctcgctgccgccgccgcaaagctgcatccttgccgctacggacgctcgtatgggcgaagtgttttatgcatggttcgatacgctgctcgagggggggcccggtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctccaccgcggtggcggccgctctagaccagccacggcgcatacaaattcacccgtccgcccacaggcgatgtattacgtatcgaccgcatcaaagaaatccaccaagccctgcccaatacacacatcgtgatgcacggctccagctccgttccgcaagaatggctgaaagtcatcaacgaatacggcggcaatatcggcgaaacctacggcgtgccggttgaagaaatcgtcgaaggcatcaaacacggcgtgcgcaaagtcaacatcgataccgacttgcgccttgcttctaccggcgcggtacgccgctaccttgccgaaaatccgtccgactttgacccgcgcaaatacctgagcaaaaccattgaggccatgaagcaaatctgcctcgaccgttatcttgcgtttggctgcgaaggtcaggcaggcaaaatcaaacctgtttcgttggaaaaaatggcaagccgttatgccaagggcgaattgaaccaaatcgtcaaataacaggttgcctgtaaacaaaatgccgtcccatgggcaaacttaagagtgtgttgatagtgcagtatcttaaaattttgtgtataataggaattgaagttaaattagatgctaaaaatttgtaattaagaaggagggattcgtcatgttggtattccaaatgcgtaatgtagataaaacatctactgttttgaaacagactaaaaacagtgattacgcagataaataaatacgttagattaattcctaccagtgactaatcttatgactttttaaacagataactaaaattacaaacaaatcgtttaacttctgtatttatttacagatgtaatcacttcaggagtaattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaacattcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccattacttatgagcaagtattgtctatttttaatagttatctattatttaacgggaggaaataattctatgagtcgcttttttaaatttggaaagttacacgttactaaagggaatggagataaattattagatatactactgacagcttccaagaagctaaagaggtccctttcgacggccccgggcctcagc

SEQ ID NO: 43 - DNA, 6673 bp, synthetic construct
  misc_feature 1..6673: note = pBS-FHbpKO-NHba plasmid
  source 1..6673: mol_type = other DNA
gcatgcatccgggcagcagcgcatcggctcgcacgaggtctgcgcttgaattgtgttgtagaaacacaacgtttttgaaaaaataagctattgttttatatcaaaatataatcatttttaaaataaaggttgcggcatttatcagatatttgttctgaaatgaagacgtatcgggtgtttgcccgatgtttttaggtttttatcaaatttacaaaaggaagcccatatgtttaaacgcagtgtgattgcaatggcttgtatttttcccctttcagcctgtgggggcggcggtggcggatcgcccgatgtcaagtcggcggacacgccgtcaaaaccggccgcccccgttgttgctgaaaatgccggggaaggggtgctgccgaaagaaaagaaagatgaggaggcagcgggcggtgcgccgcaagccgatacgcaggacgcaaccgccggagaaggcagccaagatatggcggcagtttcggcagaaaatacaggcaatggcggtgcggcaacaacggacaaccccaaaaatgaagacgcgggggcgcaaaatgatatgccgcaaaatgccgccgaatccgcaaatcaaacagggaacaaccaacccgccggttcttcagattccgcccccgcgtcaaaccctgcccctgcgaatggcggtagcgattttggaaggacgaacgtgggcaattctgttgtgattgacggaccgtcgcaaaatataacgttgacccactgtaaaggcgatccttgtaatggtgataatttattggatgaagaagcaccgccaaaatcagaatttgaatcgttaagtgatgaagagaaaattaagaaatataaaaaagatggagaaaagtttactggtttggttgctataaaagttgaaaataatgggttgaataaatataccattatttatcaagctcagcctactcgttctgcacggtcgaggaggtcgcttccggccgagattccgctgattcccgtcaatcaggccgatacgctgattgtggatggggaagcggtcagcctgacggggcattccggcaatatcttcgcgcccgaagggaattaccggtatctgacttacggggcggaaaaattgcccggcggatcgtatgccctccgtgtgcaaggcgaaccggcaaaaggcgaaatgcttgcgggcacggccgtgtacaacggcgaagtgctgcatttccatatggaaaacggccgtccgtacccgtccggaggcaggtttgccgcaaaagtcgatttcggcagcaaatctgtggacggcattatcgacagcagcgatgatttgcatatgggtacgcaaaaattcaaagccgccatcgatggaaacggctttaaggggacttggacggaaaatggcggcggggatgtttccggaaggttttacggcccggccggcgaggaagtggcgggaaaatacagctatcgcccgacagatgctgaaaagggcggattcggcgtgtttgccggcaaaaaagatcgggattgaaggcctcctgcaggccaagcaaaaccattgtgaaaatgccgtccgaacacgataatttaccgttcggacggcattttgtattgcaccgtccgacggcatgcccaaggggggaaatccctattttcaggccaaccgctatataatgccgtctgaaccaacgagagaatgccatgcaagctgattttaaccgtcccgtcctggccgtcgataccggtacttcccgtttgtcgctcgcgctgcgtgccgacggcgaaacccgtctgttccatcaggaagtcggcagccgccagtccgaactgattctgccggaaatccgcaccctattccgcgatgcaggcattaccgccgccgatttgggtgcggtcgtgtacgcacagggtcccggcgcgtttaccggactgcgtatcggcatcggtgtagctcagggtttggcaacgccgtttgatacccccttaatcggcgtaccctcgctcgatgccgccgcctcgctgccgccgccgcaaagctgcatccttgccgctacggacgctcgtatgggcgaagtgttttatgcatggttcgatacgctgctcgagggggggcccggtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctccaccgcggtggcggccgctctagaccagccacggcgcatacaaattcacccgtccgcccacaggcgatgtattacgtatcgaccgcatcaaagaaatccaccaagccctgcccaatacacacatcgtgatgcacggctccagctccgttccgcaagaatggctgaaagtcatcaacgaatacggcggcaatatcggcgaaacctacggcgtgccggttgaagaaatcgtcgaaggcatcaaacacggcgtgcgcaaagtcaacatcgataccgacttgcgccttgcttctaccggcgcggtacgccgctaccttgccgaaaatccgtccgactttgacccgcgcaaatacctgagcaaaaccattgaggccatgaagcaaatctgcctcgaccgttatcttgcgtttggctgcgaaggtcaggcaggcaaaatcaaacctgtttcgttggaaaaaatggcaagccgttatgccaagggcgaattgaaccaaatcgtcaaataacaggttgcctgtaaacaaaatgccgtcccatgggcaaacttaagagtgtgttgatagtgcagtatcttaaaattttgtgtataataggaattgaagttaaattagatgctaaaaatttgtaattaagaaggagggattcgtcatgttggtattccaaatgcgtaatgtagataaaacatctactgttttgaaacagactaaaaacagtgattacgcagataaataaatacgttagattaattcctaccagtgactaatcttatgactttttaaacagataactaaaattacaaacaaatcgtttaacttctgtatttatttacagatgtaatcacttcaggagtaattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaacattcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccattacttatgagcaagtattgtctatttttaatagttatctattatttaacgggaggaaataattctatgagtcgcttttttaaatttggaaagttacacgttactaaagggaatggagataaattattagatatactactgacagcttccaagaagctaaagaggtccctttcgacggccccgggcctcagc

SEQ ID NO: 44 - DNA, 5314 bp, synthetic construct
  misc_feature 1..5314: note = pUC18-LpxL1KO-MetQ plasmid
  source 1..5314: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttatgtgtatcgagatgaaatttatattttttgtactgtatgttttgcagtttctgccgtttgcgctgctgcacaagattgccgacctgacgggtttgcttgcctaccttctggtcaaaccgcgccgccgtatcggcgaaatcaatttggcaaaatgtttttccgaatggagtgaggaaaagcgtaaaaccgtgttgaaacagcatttcaaacacatggcgaaactgatgttggaatacggtttatattggtacgcgcctgccggacgtttgaaatcgctggtgcgctaccgcaataagcattatttggacgacgcgctggcggcgggggaaaaagtcatcatcctgtatccgcacttcaccgctgcagttctcgaggcagtgactaactaggaggaataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacccctcagcgcatgcatccgggctctgcgcttgaattgtgttgtagaaacacaacgtttttgaaaaaataagctattgttttatatcaaaatataatcatttttaaaataaaggttgcggcatttatcagatatttgttctgaaatgaagacgtatcgggtgtttgcccgatgtttttaggtttttatcaaatttacaaaaggaagcccatatgaaaaccttcttcaaaaccctttccgccgccgcactcgcgctcatcctcgcagcctgcggcggtcaaaaagacagcgcgcccgcagcctctgccgccgccccttctgccgataacggcgcggcgaaaaaagaaatcgtcttcggcacgaccgtcggcgacttcggcgatatggtcaaagaacaaatccaagccgagctggagaaaaaaggctacaccgtcaaattggtcgaatttaccgactatgtgcgcccgaatctggcattggcggagggcgagttggacatcaacgtcttccaacacaaaccctatcttgacgatttcaaaaaagaacacaacctggacatcaccgaagccttccaagtgccgaccgcgcctttgggactgtatccgggcaaactgaaatcgctggaagaagtcaaagacggcagcaccgtatccgcgcccaacgacccgtccaacttcgcacgcgccttggtgatgctgaacgaactgggttggatcaaactcaaagacggcatcaatccgctgaccgcatccaaagccgacatcgcggaaaacctgaaaaacatcaaaatcgtcgagcttgaagccgcacaactgccgcgcagccgcgccgacgtggattttgccgtcgtcaacggcaactacgccataagcagcggcatgaagctgaccgaagccctgttccaagagccgagctttgcctatgtcaactggtctgccgtcaaaaccgccgacaaagacagccaatggcttaaagacgtaaccgaggcctataactccgacgcgttcaaagcctacgcgcacaaacgcttcgagggctacaaataccctgccgcatggaatgaaggcgcagccaaataaggcagtcgtataacctgcaggcctgcagggaataatgagtcgacaggatttcggacgcaacgattcggtttttgtggattttttcggtattcagacggcaacgattaccggattgagccgcattgccgcgcttgcaaatgcaaaagtgatacccgccattcccgtccgcgaggcagacaatacggttacattgcatttctaccctgcttggaaatcctttccgggtgaagacgcgaaagccgacgcgcagcgcatgaaccgttttatcgaagacagggtgcgcgaacatccggaacaatatttttggctgcacaagcgttttaaaacccgtccggaaggcagccccgatttttactgactacgtcagacggctctagaggatccccgggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaaagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 45 - DNA, 5314 bp, synthetic construct
  misc_feature 1..5314: note = pUC18-LpxL1KO-MetQSM plasmid
  source 1..5314: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttatgtgtatcgagatgaaatttatattttttgtactgtatgttttgcagtttctgccgtttgcgctgctgcacaagattgccgacctgacgggtttgcttgcctaccttctggtcaaaccgcgccgccgtatcggcgaaatcaatttggcaaaatgtttttccgaatggagtgaggaaaagcgtaaaaccgtgttgaaacagcatttcaaacacatggcgaaactgatgttggaatacggtttatattggtacgcgcctgccggacgtttgaaatcgctggtgcgctaccgcaataagcattatttggacgacgcgctggcggcgggggaaaaagtcatcatcctgtatccgcacttcaccgctgcagttctcgaggcagtgactaactaggaggaataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacccctcagcgcatgcatccgggctctgcgcttgaattgtgttgtagaaacacaacgtttttgaaaaaataagctattgttttatatcaaaatataatcatttttaaaataaaggttgcggcatttatcagatatttgttctgaaatgaagacgtatcgggtgtttgcccgatgtttttaggtttttatcaaatttacaaaaggaagcccatatgaaaaccttcttcaaaaccctttccgccgccgcactcgcgctcatcctcgcagcctgcggcggtcaaaaagacagcgcgcccgcagcctctgccgccgccccttctgccgataacggcgcggcgaaaaaagaaatcgtcttcggcacgaccgtcggcgacttcggcgatatggtcaaagaacaaatccaagccgagctggagaaaaaaggctacaccgtcaaattggtcgaatttaccgactatgtgcgcccgaatctggcattggcggagggcgagttggacatcaacgtcttccaacacaaaccctatcttgacgatttcaaaaaagaacacaacctggacatcaccgaagccttccaagtgccgaccgcgcctttgggactgtatccgggcaaactgaaatcgctggaagaagtcaaagacggcagcaccgtatccgcgcccaacgacccgtccaacttcgcacgcgccttggtgatgctgaacgaactgggttggatcaaactcaaagacggcatcaatccgctgaccgcatccaaagccgacatcgcggaaaacctgaaaaacatcaaaatcgtcgagcttgaagccgcacaactgccgcgcagccgcgccgacgtggattttgccgtcgtcaacggcaactacgccataagcagcggcatgaagctgaccgaagccctgttccaagagccgagctttgcctatgtcgcctggtctgccgtcaaaaccgccgacaaagacagccaatggcttaaagacgtaaccgaggcctataactccgacgcgttcaaagcctacgcgcacaaacgcttcgagggctacaaataccctgccgcatggaatgaaggcgcagccaaataaggcagtcgtataacctgcaggcctgcagggaataatgagtcgacaggatttcggacgcaacgattcggtttttgtggattttttcggtattcagacggcaacgattaccggattgagccgcattgccgcgcttgcaaatgcaaaagtgatacccgccattcccgtccgcgaggcagacaatacggttacattgcatttctaccctgcttggaaatcctttccgggtgaagacgcgaaagccgacgcgcagcgcatgaaccgttttatcgaagacagggtgcgcgaacatccggaacaatatttttggctgcacaagcgttttaaaacccgtccggaaggcagccccgatttttactgactacgtcagacggctctagaggatccccgggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaaagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 46 - DNA, 5395 bp, synthetic construct
  misc_feature 1..5395: note = pUC18-LpxL1KO-GNA1220 plasmid
  source 1..5395: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttatgtgtatcgagatgaaatttatattttttgtactgtatgttttgcagtttctgccgtttgcgctgctgcacaagattgccgacctgacgggtttgcttgcctaccttctggtcaaaccgcgccgccgtatcggcgaaatcaatttggcaaaatgtttttccgaatggagtgaggaaaagcgtaaaaccgtgttgaaacagcatttcaaacacatggcgaaactgatgttggaatacggtttatattggtacgcgcctgccggacgtttgaaatcgctggtgcgctaccgcaataagcattatttggacgacgcgctggcggcgggggaaaaagtcatcatcctgtatccgcacttcaccgctgcagttctcgaggcagtgactaactaggaggaataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacccctcagcgcatgcatccgggctctgcgcttgaattgtgttgtagaaacacaacgtttttgaaaaaataagctattgttttatatcaaaatataatcatttttaaaataaaggttgcggcatttatcagatatttgttctgaaatgaagacgtatcgggtgtttgcccgatgtttttaggtttttatcaaatttacaaaaggaagcccatatggaatttttcattatcttgttggcagccgtcgccgttttcggcttcaaatcctttgtcgtcatcccccagcaggaagtccacgttgtcgaaaggctcgggcgtttccatcgcgccctgacggccggtttgaatattttgattccctttatcgaccgcgtcgcctaccgccattcgctgaaagaaatccctttagacgtacccagccaggtctgcatcacgcgcgataatacgcaattgactgttgacggcatcatctatttccaagtaaccgatcccaaactcgcctcatacggttcgagcaactacattatggcaattacccagcttgcccaaacgacgctgcgttccgttatcgggcgtatggagttggacaaaacgtttgaagaacgcgacgaaatcaacagtaccgtcgtctccgccctcgatgaagccgccggggcttggggtgtgaaagtcctccgttacgaaatcaaggatttggttccgccgcaagaaatccttcgcgcaatgcaggcacaaattaccgccgaacgcgaaaaacgcgcccgtattgccgaatccgaaggccgtaaaatcgaacaaatcaaccttgccagtggtcagcgtgaagccgaaatccaacaatccgaaggcgaggctcaggctgcggtcaatgcgtccaatgccgagaaaatcgcccgcatcaaccgcgccaaaggcgaagcggaatccctgcgccttgttgccgaagccaatgccgaagccatccgtcaaattgccgccgcccttcaaacccaaggcggggcggatgcggtcaatctgaagattgcggaacaatacgtagccgcgttcaacaatcttgccaaagaaagcaatacgctgattatgcccgccaatgttgccgacatcggcagcctgatttctgccggcatgaaaattatcgacagcagcaaaaccgccaaataaggcagtcgtataacctgcaggcctgcagggaataatgagtcgacaggatttcggacgcaacgattcggtttttgtggattttttcggtattcagacggcaacgattaccggattgagccgcattgccgcgcttgcaaatgcaaaagtgatacccgccattcccgtccgcgaggcagacaatacggttacattgcatttctaccctgcttggaaatcctttccgggtgaagacgcgaaagccgacgcgcagcgcatgaaccgttttatcgaagacagggtgcgcgaacatccggaacaatatttttggctgcacaagcgttttaaaacccgtccggaaggcagccccgatttttactgactacgtcagacggctctagaggatccccgggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaaagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 47 - DNA, 5721 bp, synthetic construct
  misc_feature 1..5721: note = pUC18-LpxL1KO-NHba plasmid
  source 1..5721: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttatgtgtatcgagatgaaatttatattttttgtactgtatgttttgcagtttctgccgtttgcgctgctgcacaagattgccgacctgacgggtttgcttgcctaccttctggtcaaaccgcgccgccgtatcggcgaaatcaatttggcaaaatgtttttccgaatggagtgaggaaaagcgtaaaaccgtgttgaaacagcatttcaaacacatggcgaaactgatgttggaatacggtttatattggtacgcgcctgccggacgtttgaaatcgctggtgcgctaccgcaataagcattatttggacgacgcgctggcggcgggggaaaaagtcatcatcctgtatccgcacttcaccgctgcagttctcgaggcagtgactaactaggaggaataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacccctcagcgcatgcatccgggctctgcgcttgaattgtgttgtagaaacacaacgtttttgaaaaaataagctattgttttatatcaaaatataatcatttttaaaataaaggttgcggcatttatcagatatttgttctgaaatgaagacgtatcgggtgtttgcccgatgtttttaggtttttatcaaatttacaaaaggaagcccatatgtttaaacgcagtgtgattgcaatggcttgtatttttcccctttcagcctgtgggggcggcggtggcggatcgcccgatgtcaagtcggcggacacgccgtcaaaaccggccgcccccgttgttgctgaaaatgccggggaaggggtgctgccgaaagaaaagaaagatgaggaggcagcgggcggtgcgccgcaagccgatacgcaggacgcaaccgccggagaaggcagccaagatatggcggcagtttcggcagaaaatacaggcaatggcggtgcggcaacaacggacaaccccaaaaatgaagacgcgggggcgcaaaatgatatgccgcaaaatgccgccgaatccgcaaatcaaacagggaacaaccaacccgccggttcttcagattccgcccccgcgtcaaaccctgcccctgcgaatggcggtagcgattttggaaggacgaacgtgggcaattctgttgtgattgacggaccgtcgcaaaatataacgttgacccactgtaaaggcgatccttgtaatggtgataatttattggatgaagaagcaccgccaaaatcagaatttgaatcgttaagtgatgaagagaaaattaagaaatataaaaaagatggagaaaagtttactggtttggttgctataaaagttgaaaataatgggttgaataaatataccattatttatcaagctcagcctactcgttctgcacggtcgaggaggtcgcttccggccgagattccgctgattcccgtcaatcaggccgatacgctgattgtggatggggaagcggtcagcctgacggggcattccggcaatatcttcgcgcccgaagggaattaccggtatctgacttacggggcggaaaaattgcccggcggatcgtatgccctccgtgtgcaaggcgaaccggcaaaaggcgaaatgcttgcgggcacggccgtgtacaacggcgaagtgctgcatttccatatggaaaacggccgtccgtacccgtccggaggcaggtttgccgcaaaagtcgatttcggcagcaaatctgtggacggcattatcgacagcagcgatgatttgcatatgggtacgcaaaaattcaaagccgccatcgatggaaacggctttaaggggacttggacggaaaatggcggcggggatgtttccggaaggttttacggcccggccggcgaggaagtggcgggaaaatacagctatcgcccgacagatgctgaaaagggcggattcggcgtgtttgccggcaaaaaagatcgggattgaaggcctcctgcaggcctgcagggaataatgagtcgacaggatttcggacgcaacgattcggtttttgtggattttttcggtattcagacggcaacgattaccggattgagccgcattgccgcgcttgcaaatgcaaaagtgatacccgccattcccgtccgcgaggcagacaatacggttacattgcatttctaccctgcttggaaatcctttccgggtgaagacgcgaaagccgacgcgcagcgcatgaaccgttttatcgaagacagggtgcgcgaacatccggaacaatatttttggctgcacaagcgttttaaaacccgtccggaaggcagccccgatttttactgactacgtcagacggctctagaggatccccgggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaaagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 48 - DNA, 6531 bp, synthetic construct
  misc_feature 1..6531: note = pGEM-SiaD-GalEKO-MetQ plasmid
  source 1..6531: mol_type = other DNA
gggcgaattcctggacacggcagaccgataaagcccgcaccgccggtaacgaggatggtttttttgtttgcggtttgcatttaatccccatatccgttggggttgcggctgacccaacgccacgaatcttccatcatttgctgcaagccgcgtttggtttcccagccggtttgttgtttggtatgggacgggtcggcaaaggaacacgccaaatcaccggcgcggcgcggtttgatttcaaatggaatggttaaacccgaagccgcttcaaatgcgcggatgatttccaataccgaagaagcgcggccggagcctaagttcagcaaatgcgtgcctgctacattactttttgcctgcatagccgcgacatggccttctgccaaatccatcacatgaatatagtcacgcatccccgtgccgtcgggggtagggtagtcatcgccaaataccgccaattgcctcagcgcatgcatccgggctctgcgcttgaattgtgttgtagaaacacaacgtttttgaaaaaataagctattgttttatatcaaaatataatcatttttaaaataaaggttgcggcatttatcagatatttgttctgaaatgaagacgtatcgggtgtttgcccgatgtttttaggtttttatcaaatttacaaaaggaagcccatatgaaaaccttcttcaaaaccctttccgccgccgcactcgcgctcatcctcgcagcctgcggcggtcaaaaagacagcgcgcccgcagcctctgccgccgccccttctgccgataacggcgcggcgaaaaaagaaatcgtcttcggcacgaccgtcggcgacttcggcgatatggtcaaagaacaaatccaagccgagctggagaaaaaaggctacaccgtcaaattggtcgaatttaccgactatgtgcgcccgaatctggcattggcggagggcgagttggacatcaacgtcttccaacacaaaccctatcttgacgatttcaaaaaagaacacaacctggacatcaccgaagccttccaagtgccgaccgcgcctttgggactgtatccgggcaaactgaaatcgctggaagaagtcaaagacggcagcaccgtatccgcgcccaacgacccgtccaacttcgcacgcgccttggtgatgctgaacgaactgggttggatcaaactcaaagacggcatcaatccgctgaccgcatccaaagccgacatcgcggaaaacctgaaaaacatcaaaatcgtcgagcttgaagccgcacaactgccgcgcagccgcgccgacgtggattttgccgtcgtcaacggcaactacgccataagcagcggcatgaagctgaccgaagccctgttccaagagccgagctttgcctatgtcaactggtctgccgtcaaaaccgccgacaaagacagccaatggcttaaagacgtaaccgaggcctataactccgacgcgttcaaagcctacgcgcacaaacgcttcgagggctacaaataccctgccgcatggaatgaaggcgcagccaaataaggcagtcgtataatctagatctagagacgatgccgtctgaagaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctatgccgtctgaatactcgagcggaacccgggtagtggcctgtaaaagacagcatatagagatgagcaggctgtataatattaaggatttttttgtaacttctataaatataaagtaattttttaggagttatattattagggcttctaggaagctcaaatagataaatagattcaaatagattcttgttagctgattgatgaactaacttaggcatttttaagtttttagaagtatataaaattactagtaaattattggttaatttttgtattttaattaggctttggacttggttaagctgacctaaattagatatgacaaataaattgttacgtgggggggtaagataaaatggagatgttgtcaaccacattgaatcttgaaaaaactttttaggctgaaaaagagctttttttattttctttagcattattgtatctcttaaaaattaatgagaattagctatatgtaatagccaatcctctgttaataaagtaactaagttaataagcattattcaatatcagtttttttgatttgagcacctttgcgaatattgcaagcagcgaccttaccaaataatgtttcatattcgttgacgctgaagtctccattgcctgggcgtttaacccataggttatctaagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactata

SEQ ID NO: 49 - DNA, 6531 bp, synthetic construct
  misc_feature 1..6531: note = pGEM-SiaD-GlaEKO-MetQSM plasmid
  source 1..6531: mol_type = other DNA
gggcgaattcctggacacggcagaccgataaagcccgcaccgccggtaacgaggatggtttttttgtttgcggtttgcatttaatccccatatccgttggggttgcggctgacccaacgccacgaatcttccatcatttgctgcaagccgcgtttggtttcccagccggtttgttgtttggtatgggacgggtcggcaaaggaacacgccaaatcaccggcgcggcgcggtttgatttcaaatggaatggttaaacccgaagccgcttcaaatgcgcggatgatttccaataccgaagaagcgcggccggagcctaagttcagcaaatgcgtgcctgctacattactttttgcctgcatagccgcgacatggccttctgccaaatccatcacatgaatatagtcacgcatccccgtgccgtcgggggtagggtagtcatcgccaaataccgccaattgcctcagcgcatgcatccgggctctgcgcttgaattgtgttgtagaaacacaacgtttttgaaaaaataagctattgttttatatcaaaatataatcatttttaaaataaaggttgcggcatttatcagatatttgttctgaaatgaagacgtatcgggtgtttgcccgatgtttttaggtttttatcaaatttacaaaaggaagcccatatgaaaaccttcttcaaaaccctttccgccgccgcactcgcgctcatcctcgcagcctgcggcggtcaaaaagacagcgcgcccgcagcctctgccgccgccccttctgccgataacggcgcggcgaaaaaagaaatcgtcttcggcacgaccgtcggcgacttcggcgatatggtcaaagaacaaatccaagccgagctggagaaaaaaggctacaccgtcaaattggtcgaatttaccgactatgtgcgcccgaatctggcattggcggagggcgagttggacatcaacgtcttccaacacaaaccctatcttgacgatttcaaaaaagaacacaacctggacatcaccgaagccttccaagtgccgaccgcgcctttgggactgtatccgggcaaactgaaatcgctggaagaagtcaaagacggcagcaccgtatccgcgcccaacgacccgtccaacttcgcacgcgccttggtgatgctgaacgaactgggttggatcaaactcaaagacggcatcaatccgctgaccgcatccaaagccgacatcgcggaaaacctgaaaaacatcaaaatcgtcgagcttgaagccgcacaactgccgcgcagccgcgccgacgtggattttgccgtcgtcaacggcaactacgccataagcagcggcatgaagctgaccgaagccctgttccaagagccgagctttgcctatgtcgcctggtctgccgtcaaaaccgccgacaaagacagccaatggcttaaagacgtaaccgaggcctataactccgacgcgttcaaagcctacgcgcacaaacgcttcgagggctacaaataccctgccgcatggaatgaaggcgcagccaaataaggcagtcgtataatctagatctagagacgatgccgtctgaagaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctatgccgtctgaatactcgagcggaacccgggtagtggcctgtaaaagacagcatatagagatgagcaggctgtataatattaaggatttttttgtaacttctataaatataaagtaattttttaggagttatattattagggcttctaggaagctcaaatagataaatagattcaaatagattcttgttagctgattgatgaactaacttaggcatttttaagtttttagaagtatataaaattactagtaaattattggttaatttttgtattttaattaggctttggacttggttaagctgacctaaattagatatgacaaataaattgttacgtgggggggtaagataaaatggagatgttgtcaaccacattgaatcttgaaaaaactttttaggctgaaaaagagctttttttattttctttagcattattgtatctcttaaaaattaatgagaattagctatatgtaatagccaatcctctgttaataaagtaactaagttaataagcattattcaatatcagtttttttgatttgagcacctttgcgaatattgcaagcagcgaccttaccaaataatgtttcatattcgttgacgctgaagtctccattgcctgggcgtttaacccataggttatctaagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactata

SEQ ID NO: 50 - DNA, 6612 bp, synthetic construct
  misc_feature 1..6612: note = pGEM-SiaD-GlaEKO-GNA1220 plasmid
  source 1..6612: mol_type = other DNA
gggcgaattcctggacacggcagaccgataaagcccgcaccgccggtaacgaggatggtttttttgtttgcggtttgcatttaatccccatatccgttggggttgcggctgacccaacgccacgaatcttccatcatttgctgcaagccgcgtttggtttcccagccggtttgttgtttggtatgggacgggtcggcaaaggaacacgccaaatcaccggcgcggcgcggtttgatttcaaatggaatggttaaacccgaagccgcttcaaatgcgcggatgatttccaataccgaagaagcgcggccggagcctaagttcagcaaatgcgtgcctgctacattactttttgcctgcatagccgcgacatggccttctgccaaatccatcacatgaatatagtcacgcatccccgtgccgtcgggggtagggtagtcatcgccaaataccgccaattgcctcagcgcatgcatccgggctctgcgcttgaattgtgttgtagaaacacaacgtttttgaaaaaataagctattgttttatatcaaaatataatcatttttaaaataaaggttgcggcatttatcagatatttgttctgaaatgaagacgtatcgggtgtttgcccgatgtttttaggtttttatcaaatttacaaaaggaagcccatatggaatttttcattatcttgttggcagccgtcgccgttttcggcttcaaatcctttgtcgtcatcccccagcaggaagtccacgttgtcgaaaggctcgggcgtttccatcgcgccctgacggccggtttgaatattttgattccctttatcgaccgcgtcgcctaccgccattcgctgaaagaaatccctttagacgtacccagccaggtctgcatcacgcgcgataatacgcaattgactgttgacggcatcatctatttccaagtaaccgatcccaaactcgcctcatacggttcgagcaactacattatggcaattacccagcttgcccaaacgacgctgcgttccgttatcgggcgtatggagttggacaaaacgtttgaagaacgcgacgaaatcaacagtaccgtcgtctccgccctcgatgaagccgccggggcttggggtgtgaaagtcctccgttacgaaatcaaggatttggttccgccgcaagaaatccttcgcgcaatgcaggcacaaattaccgccgaacgcgaaaaacgcgcccgtattgccgaatccgaaggccgtaaaatcgaacaaatcaaccttgccagtggtcagcgtgaagccgaaatccaacaatccgaaggcgaggctcaggctgcggtcaatgcgtccaatgccgagaaaatcgcccgcatcaaccgcgccaaaggcgaagcggaatccctgcgccttgttgccgaagccaatgccgaagccatccgtcaaattgccgccgcccttcaaacccaaggcggggcggatgcggtcaatctgaagattgcggaacaatacgtagccgcgttcaacaatcttgccaaagaaagcaatacgctgattatgcccgccaatgttgccgacatcggcagcctgatttctgccggcatgaaaattatcgacagcagcaaaaccgccaaataaggcagtcgtataatctagatctagagacgatgccgtctgaagaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctatgccgtctgaatactcgagcggaacccgggtagtggcctgtaaaagacagcatatagagatgagcaggctgtataatattaaggatttttttgtaacttctataaatataaagtaattttttaggagttatattattagggcttctaggaagctcaaatagataaatagattcaaatagattcttgttagctgattgatgaactaacttaggcatttttaagtttttagaagtatataaaattactagtaaattattggttaatttttgtattttaattaggctttggacttggttaagctgacctaaattagatatgacaaataaattgttacgtgggggggtaagataaaatggagatgttgtcaaccacattgaatcttgaaaaaactttttaggctgaaaaagagctttttttattttctttagcattattgtatctcttaaaaattaatgagaattagctatatgtaatagccaatcctctgttaataaagtaactaagttaataagcattattcaatatcagtttttttgatttgagcacctttgcgaatattgcaagcagcgaccttaccaaataatgtttcatattcgttgacgctgaagtctccattgcctgggcgtttaacccataggttatctaagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactata

SEQ ID NO: 51 - DNA, 6938 bp, synthetic construct
  misc_feature 1..6938: note = pGEM-SiaD-GlaEKO-NHba plasmid
  source 1..6938: mol_type = other DNA
gggcgaattcctggacacggcagaccgataaagcccgcaccgccggtaacgaggatggtttttttgtttgcggtttgcatttaatccccatatccgttggggttgcggctgacccaacgccacgaatcttccatcatttgctgcaagccgcgtttggtttcccagccggtttgttgtttggtatgggacgggtcggcaaaggaacacgccaaatcaccggcgcggcgcggtttgatttcaaatggaatggttaaacccgaagccgcttcaaatgcgcggatgatttccaataccgaagaagcgcggccggagcctaagttcagcaaatgcgtgcctgctacattactttttgcctgcatagccgcgacatggccttctgccaaatccatcacatgaatatagtcacgcatccccgtgccgtcgggggtagggtagtcatcgccaaataccgccaattgcctcagcgcatgcatccgggctctgcgcttgaattgtgttgtagaaacacaacgtttttgaaaaaataagctattgttttatatcaaaatataatcatttttaaaataaaggttgcggcatttatcagatatttgttctgaaatgaagacgtatcgggtgtttgcccgatgtttttaggtttttatcaaatttacaaaaggaagcccatatgtttaaacgcagtgtgattgcaatggcttgtatttttcccctttcagcctgtgggggcggcggtggcggatcgcccgatgtcaagtcggcggacacgccgtcaaaaccggccgcccccgttgttgctgaaaatgccggggaaggggtgctgccgaaagaaaagaaagatgaggaggcagcgggcggtgcgccgcaagccgatacgcaggacgcaaccgccggagaaggcagccaagatatggcggcagtttcggcagaaaatacaggcaatggcggtgcggcaacaacggacaaccccaaaaatgaagacgcgggggcgcaaaatgatatgccgcaaaatgccgccgaatccgcaaatcaaacagggaacaaccaacccgccggttcttcagattccgcccccgcgtcaaaccctgcccctgcgaatggcggtagcgattttggaaggacgaacgtgggcaattctgttgtgattgacggaccgtcgcaaaatataacgttgacccactgtaaaggcgatccttgtaatggtgataatttattggatgaagaagcaccgccaaaatcagaatttgaatcgttaagtgatgaagagaaaattaagaaatataaaaaagatggagaaaagtttactggtttggttgctataaaagttgaaaataatgggttgaataaatataccattatttatcaagctcagcctactcgttctgcacggtcgaggaggtcgcttccggccgagattccgctgattcccgtcaatcaggccgatacgctgattgtggatggggaagcggtcagcctgacggggcattccggcaatatcttcgcgcccgaagggaattaccggtatctgacttacggggcggaaaaattgcccggcggatcgtatgccctccgtgtgcaaggcgaaccggcaaaaggcgaaatgcttgcgggcacggccgtgtacaacggcgaagtgctgcatttccatatggaaaacggccgtccgtacccgtccggaggcaggtttgccgcaaaagtcgatttcggcagcaaatctgtggacggcattatcgacagcagcgatgatttgcatatgggtacgcaaaaattcaaagccgccatcgatggaaacggctttaaggggacttggacggaaaatggcggcggggatgtttccggaaggttttacggcccggccggcgaggaagtggcgggaaaatacagctatcgcccgacagatgctgaaaagggcggattcggcgtgtttgccggcaaaaaagatcgggattgaaggccttctagatctagagacgatgccgtctgaagaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctatgccgtctgaatactcgagcggaacccgggtagtggcctgtaaaagacagcatatagagatgagcaggctgtataatattaaggatttttttgtaacttctataaatataaagtaattttttaggagttatattattagggcttctaggaagctcaaatagataaatagattcaaatagattcttgttagctgattgatgaactaacttaggcatttttaagtttttagaagtatataaaattactagtaaattattggttaatttttgtattttaattaggctttggacttggttaagctgacctaaattagatatgacaaataaattgttacgtgggggggtaagataaaatggagatgttgtcaaccacattgaatcttgaaaaaactttttaggctgaaaaagagctttttttattttctttagcattattgtatctcttaaaaattaatgagaattagctatatgtaatagccaatcctctgttaataaagtaactaagttaataagcattattcaatatcagtttttttgatttgagcacctttgcgaatattgcaagcagcgaccttaccaaataatgtttcatattcgttgacgctgaagtctccattgcctgggcgtttaacccataggttatctaagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactata

SEQ ID NO: 52 - DNA, 7659 bp, synthetic construct
  misc_feature 1..7659: note = pFP12-MetQ plasmid
  source 1..7659: mol_type = other DNA
ctgcaggccgtctgaaccgctctaaccgctttttctcggcttaatttttctgtctctgttataaaattgctattcatcttgttcttcttcaaaaaaaagttaagtaaaatacctacctaaatttttactagttcgcaatctacgagcttataacctcgttttttcaattcatttaaaaaatcagattttgagcctaattttgatctattgctatcgttacccgctagaaatacccagtaattacgcaaatcttcattggtaactttcgtaatatcggtgtaatgatcttcgagtatttttaagcaatctctagcccataaaccgtactcgtgattgctcatcttagggttttgcttatcgagtttgacgaacttcccatacttgtttttatgtggaaatactggccgttttgcaacttcttcaattttttgagctgttcgttttttactaccaatcacaaaatttaaagagtgaatagtacgcccacgcttgatttgttcaacctcaacgactaaatcagatttctcgttaatctcagttattgcaggttccaaaacacgttgatttaatgaattaaatctagggtatttattttcaacctgaagccattcttttagtttttctactgtaatttcacgactaccaacagagcgatattgtgtaattagctcataaattcgaattgaatgtacactgttgaaataagcgatatgtttgagttgatattgcgtgaattgccctttaagttgcgttaggtatggcataacttcatcagtcattgcaattctaaaacgcccctctttcttgaaatatgttctagaggaaacccaacgaaattcagttacacggtctttatcttcagttttaacacttcggtcgtaaatccgttttatagccgcctgaatttgcttataggcgttatcttggcttatttctggaaactcacggacaaaatcagccaccgtaaaatcaaaaatcttttgattagatttcggatccatagtcccgatagttaaagctaaaattctgatttcatcaatactcaatcggtaattggcttcaataaggctattagcctttacaacaactaaatcatttggcataagacaacaaatttcctgtttaaaacaacaagcaaaatatacctgttgtttatatataaaacaacaagtattttcttaaaagttgtctataacaggaaatttgttgtcgtataacaggaaatttgttgtcttataacaggaaatttgttgtcgtataacaggaaatttgttgtcgtataagtttgtaacttattgattttactggttttaaaaacgccgaaaacaagtaaaaaacaaaaatataaaaatatagggactttcgtcccttttttgggctttcagccctaattttttcttttttcaggattaaaaattacaaaacccttacagagcaagtaaacttgtttgcttgttctgcaagggttcagcaaccgtagccgtcaggcgtagggcggtagcctataaaagccatttaattttatctttaaacttccttttaaatgctttgagtgggtgtcttttatcgtactcatcaatccttttttgcattctttcgtttgctttgtgatcggcaaattttgaataagatttttccatctcatctaacattctatcaatccgttttttatgttgccatttcaggtaaacataaacacttatagcaattaaagacaatatcaatacattgtaaaaaatgattgttacaatttcgctcacagttattttttacctttttcaatttcttcattgataaatgcactcaattcatcaaatttcttgtcatcattgataaatttacgcaacttagggaagtttctatctacatctaaaagagggctatttattacttcatttagccaaaaagcccctaataaaaccttgtaatgcgtagctttcttacgcttttctgcttgttcttttgacttaatcgcacgaattttcgctttgatttcgtcctgcttgcgttgtaaatctgcttgttgctgttccaatcttgtaagtttttcgcttgccatactagcccctttatatagttagaaattatcgttattttattcagtaggtgctaggcttgcaagtgttctgttcattacgttaaaataacgtaatgcccacttatcagtttctcttcgagaaactggtgggcaagcgtaccgcttgaccgtttcgcaatactcaacactatggcaatctatcatttaaacgttcgctattgcagtaaaagcaaagggcaatcagctcaagccaaaaacgactacatcaaccgcaatgataaatattcaaagcggttagatgatttacagttttcaggctatggtaatatgccaaaatttgccgaagataatccgcaagaattttggcgattgtcagatatttacgagcgagctaatgcccgagtttgtactgaaattgaatttgctttacctagagaattaaccctagaacaacagcaaaaattagtaagttcgtttatagaaaatacggttgatagcggtagcaataaactaccctactctttcgctatccataccgataaaaataatcataatccccattgtcatttgatattttcagaacgccaacttgacggcatagaccgtacagccgagcagttttttaaacgtgctaatactaaatccccagaaaagggcggagcgatgaaaacggcagattttcgagatcgtgagtttatccaatctgtccgaaaaacgtggagagagcaagctaatcaagccttagagcaatacggatatgccgcacgaattgacgaacgtagctacaaggaacaaggcatagagcaagccccaagagcaagaattgacagggtaacgtggcaagaattgaaccgattagagcaagaagaacgccaaatcgtgcaagagcttgcacttaaaggacaagaaattaacaaagaaaaatcctacttgcagaaaatcgaagaaaaacaggctcaaggaatgggcaaatatgaatccaaattcgcagctgcgttttctaaattatcggaaagtgccctaaaacacgatttaagcaacgaaaaagaaaaagacagtaaaatacacactcaagaagaaaaagtgcctcaaaatcgcattcaggggctttctcaagcagattttgatcagtttttaattgatgaatggctacctcaaatagaaaaatacgttaaagcccaagaaaagcgggacggaatggaagtagagatcacgcaatacgacaaggatttacagcgtattcagggagactataacaagctcacagataaaaatcagggttttctcggtttatgggaaactaaagagcaaaaagcaaagaaaaaagagcttgaagatgaatacaaacatacagcagagcaacggaacgctaaaagccaagaattagccgagtatagccaaaaaataaaagcatacgaacagaaaacgctagagccaatcaacgagaagattgccaaatatcaagctgacaaccctgaaataaaaatgcggagcttaggatttgtgaaaaaaattaaggctcgaggggcatataaagcggctcaagagcgaatggagcgagaaaaacagcaccaacaggaaaaacaacagagacatttagagcgagagagtggtttgagcttgtagctaacgccctacgcctacggcttcggttgttcaacccttaaagaactcgcaacaagttgcaaattctttaagggttcgcaataaaaacaaccgctaaacatttctgcccagcggttgaaaatttacctattcaccattacaatgatcaagcaggaaatttttttgattgccgtaaatgtccgtatatctagttgaggcacaacccgccaaagtcattgccccaaccagaacggcgaaagctaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacgggaaacagctatgaccatgattacgccaagcttcctcagcgcatgcatccgggcagcagcgcatcggctcgcacgaggtctgcgcttgaattgtgttgtagaaacacaacgtttttgaaaaaataagctattgttttatatcaaaatataatcatttttaaaataaaggttgcggcatttatcagatatttgttctgaaatgaagacgtatcgggtgtttgcccgatgtttttaggtttttatcaaatttacaaaaggaagcccatatgaaaaccttcttcaaaaccctttccgccgccgcactcgcgctcatcctcgcagcctgcggcggtcaaaaagacagcgcgcccgcagcctctgccgccgccccttctgccgataacggcgcggcgaaaaaagaaatcgtcttcggcacgaccgtcggcgacttcggcgatatggtcaaagaacaaatccaagccgagctggagaaaaaaggctacaccgtcaaattggtcgaatttaccgactatgtgcgcccgaatctggcattggcggagggcgagttggacatcaacgtcttccaacacaaaccctatcttgacgatttcaaaaaagaacacaacctggacatcaccgaagccttccaagtgccgaccgcgcctttgggactgtatccgggcaaactgaaatcgctggaagaagtcaaagacggcagcaccgtatccgcgcccaacgacccgtccaacttcgcacgcgccttggtgatgctgaacgaactgggttggatcaaactcaaagacggcatcaatccgctgaccgcatccaaagccgacatcgcggaaaacctgaaaaacatcaaaatcgtcgagcttgaagccgcacaactgccgcgcagccgcgccgacgtggattttgccgtcgtcaacggcaactacgccataagcagcggcatgaagctgaccgaagccctgttccaagagccgagctttgcctatgtcaactggtctgccgtcaaaaccgccgacaaagacagccaatggcttaaagacgtaaccgaggcctataactccgacgcgttcaaagcctacgcgcacaaacgcttcgagggctacaaataccctgccgcatggaatgaaggcgcagccaaataaggcagtcgtataacctgcaggactagtcggccgtacctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtgggccctttcgtctcgcgcgtttcggtgatagcttaagtgcggtcatcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaaca

SEQ ID NO: 53 - DNA, 7659 bp, synthetic construct
  misc_feature 1..7659: note = pFP12-MetQSM plasmid
  source 1..7659: mol_type = other DNA
ctgcaggccgtctgaaccgctctaaccgctttttctcggcttaatttttctgtctctgttataaaattgctattcatcttgttcttcttcaaaaaaaagttaagtaaaatacctacctaaatttttactagttcgcaatctacgagcttataacctcgttttttcaattcatttaaaaaatcagattttgagcctaattttgatctattgctatcgttacccgctagaaatacccagtaattacgcaaatcttcattggtaactttcgtaatatcggtgtaatgatcttcgagtatttttaagcaatctctagcccataaaccgtactcgtgattgctcatcttagggttttgcttatcgagtttgacgaacttcccatacttgtttttatgtggaaatactggccgttttgcaacttcttcaattttttgagctgttcgttttttactaccaatcacaaaatttaaagagtgaatagtacgcccacgcttgatttgttcaacctcaacgactaaatcagatttctcgttaatctcagttattgcaggttccaaaacacgttgatttaatgaattaaatctagggtatttattttcaacctgaagccattcttttagtttttctactgtaatttcacgactaccaacagagcgatattgtgtaattagctcataaattcgaattgaatgtacactgttgaaataagcgatatgtttgagttgatattgcgtgaattgccctttaagttgcgttaggtatggcataacttcatcagtcattgcaattctaaaacgcccctctttcttgaaatatgttctagaggaaacccaacgaaattcagttacacggtctttatcttcagttttaacacttcggtcgtaaatccgttttatagccgcctgaatttgcttataggcgttatcttggcttatttctggaaactcacggacaaaatcagccaccgtaaaatcaaaaatcttttgattagatttcggatccatagtcccgatagttaaagctaaaattctgatttcatcaatactcaatcggtaattggcttcaataaggctattagcctttacaacaactaaatcatttggcataagacaacaaatttcctgtttaaaacaacaagcaaaatatacctgttgtttatatataaaacaacaagtattttcttaaaagttgtctataacaggaaatttgttgtcgtataacaggaaatttgttgtcttataacaggaaatttgttgtcgtataacaggaaatttgttgtcgtataagtttgtaacttattgattttactggttttaaaaacgccgaaaacaagtaaaaaacaaaaatataaaaatatagggactttcgtcccttttttgggctttcagccctaattttttcttttttcaggattaaaaattacaaaacccttacagagcaagtaaacttgtttgcttgttctgcaagggttcagcaaccgtagccgtcaggcgtagggcggtagcctataaaagccatttaattttatctttaaacttccttttaaatgctttgagtgggtgtcttttatcgtactcatcaatccttttttgcattctttcgtttgctttgtgatcggcaaattttgaataagatttttccatctcatctaacattctatcaatccgttttttatgttgccatttcaggtaaacataaacacttatagcaattaaagacaatatcaatacattgtaaaaaatgattgttacaatttcgctcacagttattttttacctttttcaatttcttcattgataaatgcactcaattcatcaaatttcttgtcatcattgataaatttacgcaacttagggaagtttctatctacatctaaaagagggctatttattacttcatttagccaaaaagcccctaataaaaccttgtaatgcgtagctttcttacgcttttctgcttgttcttttgacttaatcgcacgaattttcgctttgatttcgtcctgcttgcgttgtaaatctgcttgttgctgttccaatcttgtaagtttttcgcttgccatactagcccctttatatagttagaaattatcgttattttattcagtaggtgctaggcttgcaagtgttctgttcattacgttaaaataacgtaatgcccacttatcagtttctcttcgagaaactggtgggcaagcgtaccgcttgaccgtttcgcaatactcaacactatggcaatctatcatttaaacgttcgctattgcagtaaaagcaaagggcaatcagctcaagccaaaaacgactacatcaaccgcaatgataaatattcaaagcggttagatgatttacagttttcaggctatggtaatatgccaaaatttgccgaagataatccgcaagaattttggcgattgtcagatatttacgagcgagctaatgcccgagtttgtactgaaattgaatttgctttacctagagaattaaccctagaacaacagcaaaaattagtaagttcgtttatagaaaatacggttgatagcggtagcaataaactaccctactctttcgctatccataccgataaaaataatcataatccccattgtcatttgatattttcagaacgccaacttgacggcatagaccgtacagccgagcagttttttaaacgtgctaatactaaatccccagaaaagggcggagcgatgaaaacggcagattttcgagatcgtgagtttatccaatctgtccgaaaaacgtggagagagcaagctaatcaagccttagagcaatacggatatgccgcacgaattgacgaacgtagctacaaggaacaaggcatagagcaagccccaagagcaagaattgacagggtaacgtggcaagaattgaaccgattagagcaagaagaacgccaaatcgtgcaagagcttgcacttaaaggacaagaaattaacaaagaaaaatcctacttgcagaaaatcgaagaaaaacaggctcaaggaatgggcaaatatgaatccaaattcgcagctgcgttttctaaattatcggaaagtgccctaaaacacgatttaagcaacgaaaaagaaaaagacagtaaaatacacactcaagaagaaaaagtgcctcaaaatcgcattcaggggctttctcaagcagattttgatcagtttttaattgatgaatggctacctcaaatagaaaaatacgttaaagcccaagaaaagcgggacggaatggaagtagagatcacgcaatacgacaaggatttacagcgtattcagggagactataacaagctcacagataaaaatcagggttttctcggtttatgggaaactaaagagcaaaaagcaaagaaaaaagagcttgaagatgaatacaaacatacagcagagcaacggaacgctaaaagccaagaattagccgagtatagccaaaaaataaaagcatacgaacagaaaacgctagagccaatcaacgagaagattgccaaatatcaagctgacaaccctgaaataaaaatgcggagcttaggatttgtgaaaaaaattaaggctcgaggggcatataaagcggctcaagagcgaatggagcgagaaaaacagcaccaacaggaaaaacaacagagacatttagagcgagagagtggtttgagcttgtagctaacgccctacgcctacggcttcggttgttcaacccttaaagaactcgcaacaagttgcaaattctttaagggttcgcaataaaaacaaccgctaaacatttctgcccagcggttgaaaatttacctattcaccattacaatgatcaagcaggaaatttttttgattgccgtaaatgtccgtatatctagttgaggcacaacccgccaaagtcattgccccaaccagaacggcgaaagctaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacgggaaacagctatgaccatgattacgccaagcttcctcagcgcatgcatccgggcagcagcgcatcggctcgcacgaggtctgcgcttgaattgtgttgtagaaacacaacgtttttgaaaaaataagctattgttttatatcaaaatataatcatttttaaaataaaggttgcggcatttatcagatatttgttctgaaatgaagacgtatcgggtgtttgcccgatgtttttaggtttttatcaaatttacaaaaggaagcccatatgaaaaccttcttcaaaaccctttccgccgccgcactcgcgctcatcctcgcagcctgcggcggtcaaaaagacagcgcgcccgcagcctctgccgccgccccttctgccgataacggcgcggcgaaaaaagaaatcgtcttcggcacgaccgtcggcgacttcggcgatatggtcaaagaacaaatccaagccgagctggagaaaaaaggctacaccgtcaaattggtcgaatttaccgactatgtgcgcccgaatctggcattggcggagggcgagttggacatcaacgtcttccaacacaaaccctatcttgacgatttcaaaaaagaacacaacctggacatcaccgaagccttccaagtgccgaccgcgcctttgggactgtatccgggcaaactgaaatcgctggaagaagtcaaagacggcagcaccgtatccgcgcccaacgacccgtccaacttcgcacgcgccttggtgatgctgaacgaactgggttggatcaaactcaaagacggcatcaatccgctgaccgcatccaaagccgacatcgcggaaaacctgaaaaacatcaaaatcgtcgagcttgaagccgcacaactgccgcgcagccgcgccgacgtggattttgccgtcgtcaacggcaactacgccataagcagcggcatgaagctgaccgaagccctgttccaagagccgagctttgcctatgtcgcctggtctgccgtcaaaaccgccgacaaagacagccaatggcttaaagacgtaaccgaggcctataactccgacgcgttcaaagcctacgcgcacaaacgcttcgagggctacaaataccctgccgcatggaatgaaggcgcagccaaataaggcagtcgtataacctgcaggactagtcggccgtacctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtgggccctttcgtctcgcgcgtttcggtgatagcttaagtgcggtcatcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaaca

SEQ ID NO: 54 - DNA, 7740 bp, synthetic construct
  misc_feature 1..7740: note = pFP12-GNA1220 plasmid
  source 1..7740: mol_type = other DNA
ctgcaggccgtctgaaccgctctaaccgctttttctcggcttaatttttctgtctctgttataaaattgctattcatcttgttcttcttcaaaaaaaagttaagtaaaatacctacctaaatttttactagttcgcaatctacgagcttataacctcgttttttcaattcatttaaaaaatcagattttgagcctaattttgatctattgctatcgttacccgctagaaatacccagtaattacgcaaatcttcattggtaactttcgtaatatcggtgtaatgatcttcgagtatttttaagcaatctctagcccataaaccgtactcgtgattgctcatcttagggttttgcttatcgagtttgacgaacttcccatacttgtttttatgtggaaatactggccgttttgcaacttcttcaattttttgagctgttcgttttttactaccaatcacaaaatttaaagagtgaatagtacgcccacgcttgatttgttcaacctcaacgactaaatcagatttctcgttaatctcagttattgcaggttccaaaacacgttgatttaatgaattaaatctagggtatttattttcaacctgaagccattcttttagtttttctactgtaatttcacgactaccaacagagcgatattgtgtaattagctcataaattcgaattgaatgtacactgttgaaataagcgatatgtttgagttgatattgcgtgaattgccctttaagttgcgttaggtatggcataacttcatcagtcattgcaattctaaaacgcccctctttcttgaaatatgttctagaggaaacccaacgaaattcagttacacggtctttatcttcagttttaacacttcggtcgtaaatccgttttatagccgcctgaatttgcttataggcgttatcttggcttatttctggaaactcacggacaaaatcagccaccgtaaaatcaaaaatcttttgattagatttcggatccatagtcccgatagttaaagctaaaattctgatttcatcaatactcaatcggtaattggcttcaataaggctattagcctttacaacaactaaatcatttggcataagacaacaaatttcctgtttaaaacaacaagcaaaatatacctgttgtttatatataaaacaacaagtattttcttaaaagttgtctataacaggaaatttgttgtcgtataacaggaaatttgttgtcttataacaggaaatttgttgtcgtataacaggaaatttgttgtcgtataagtttgtaacttattgattttactggttttaaaaacgccgaaaacaagtaaaaaacaaaaatataaaaatatagggactttcgtcccttttttgggctttcagccctaattttttcttttttcaggattaaaaattacaaaacccttacagagcaagtaaacttgtttgcttgttctgcaagggttcagcaaccgtagccgtcaggcgtagggcggtagcctataaaagccatttaattttatctttaaacttccttttaaatgctttgagtgggtgtcttttatcgtactcatcaatccttttttgcattctttcgtttgctttgtgatcggcaaattttgaataagatttttccatctcatctaacattctatcaatccgttttttatgttgccatttcaggtaaacataaacacttatagcaattaaagacaatatcaatacattgtaaaaaatgattgttacaatttcgctcacagttattttttacctttttcaatttcttcattgataaatgcactcaattcatcaaatttcttgtcatcattgataaatttacgcaacttagggaagtttctatctacatctaaaagagggctatttattacttcatttagccaaaaagcccctaataaaaccttgtaatgcgtagctttcttacgcttttctgcttgttcttttgacttaatcgcacgaattttcgctttgatttcgtcctgcttgcgttgtaaatctgcttgttgctgttccaatcttgtaagtttttcgcttgccatactagcccctttatatagttagaaattatcgttattttattcagtaggtgctaggcttgcaagtgttctgttcattacgttaaaataacgtaatgcccacttatcagtttctcttcgagaaactggtgggcaagcgtaccgcttgaccgtttcgcaatactcaacactatggcaatctatcatttaaacgttcgctattgcagtaaaagcaaagggcaatcagctcaagccaaaaacgactacatcaaccgcaatgataaatattcaaagcggttagatgatttacagttttcaggctatggtaatatgccaaaatttgccgaagataatccgcaagaattttggcgattgtcagatatttacgagcgagctaatgcccgagtttgtactgaaattgaatttgctttacctagagaattaaccctagaacaacagcaaaaattagtaagttcgtttatagaaaatacggttgatagcggtagcaataaactaccctactctttcgctatccataccgataaaaataatcataatccccattgtcatttgatattttcagaacgccaacttgacggcatagaccgtacagccgagcagttttttaaacgtgctaatactaaatccccagaaaagggcggagcgatgaaaacggcagattttcgagatcgtgagtttatccaatctgtccgaaaaacgtggagagagcaagctaatcaagccttagagcaatacggatatgccgcacgaattgacgaacgtagctacaaggaacaaggcatagagcaagccccaagagcaagaattgacagggtaacgtggcaagaattgaaccgattagagcaagaagaacgccaaatcgtgcaagagcttgcacttaaaggacaagaaattaacaaagaaaaatcctacttgcagaaaatcgaagaaaaacaggctcaaggaatgggcaaatatgaatccaaattcgcagctgcgttttctaaattatcggaaagtgccctaaaacacgatttaagcaacgaaaaagaaaaagacagtaaaatacacactcaagaagaaaaagtgcctcaaaatcgcattcaggggctttctcaagcagattttgatcagtttttaattgatgaatggctacctcaaatagaaaaatacgttaaagcccaagaaaagcgggacggaatggaagtagagatcacgcaatacgacaaggatttacagcgtattcagggagactataacaagctcacagataaaaatcagggttttctcggtttatgggaaactaaagagcaaaaagcaaagaaaaaagagcttgaagatgaatacaaacatacagcagagcaacggaacgctaaaagccaagaattagccgagtatagccaaaaaataaaagcatacgaacagaaaacgctagagccaatcaacgagaagattgccaaatatcaagctgacaaccctgaaataaaaatgcggagcttaggatttgtgaaaaaaattaaggctcgaggggcatataaagcggctcaagagcgaatggagcgagaaaaacagcaccaacaggaaaaacaacagagacatttagagcgagagagtggtttgagcttgtagctaacgccctacgcctacggcttcggttgttcaacccttaaagaactcgcaacaagttgcaaattctttaagggttcgcaataaaaacaaccgctaaacatttctgcccagcggttgaaaatttacctattcaccattacaatgatcaagcaggaaatttttttgattgccgtaaatgtccgtatatctagttgaggcacaacccgccaaagtcattgccccaaccagaacggcgaaagctaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacgggaaacagctatgaccatgattacgccaagcttcctcagcgcatgcatccgggcagcagcgcatcggctcgcacgaggtctgcgcttgaattgtgttgtagaaacacaacgtttttgaaaaaataagctattgttttatatcaaaatataatcatttttaaaataaaggttgcggcatttatcagatatttgttctgaaatgaagacgtatcgggtgtttgcccgatgtttttaggtttttatcaaatttacaaaaggaagcccatatggaatttttcattatcttgttggcagccgtcgccgttttcggcttcaaatcctttgtcgtcatcccccagcaggaagtccacgttgtcgaaaggctcgggcgtttccatcgcgccctgacggccggtttgaatattttgattccctttatcgaccgcgtcgcctaccgccattcgctgaaagaaatccctttagacgtacccagccaggtctgcatcacgcgcgataatacgcaattgactgttgacggcatcatctatttccaagtaaccgatcccaaactcgcctcatacggttcgagcaactacattatggcaattacccagcttgcccaaacgacgctgcgttccgttatcgggcgtatggagttggacaaaacgtttgaagaacgcgacgaaatcaacagtaccgtcgtctccgccctcgatgaagccgccggggcttggggtgtgaaagtcctccgttacgaaatcaaggatttggttccgccgcaagaaatccttcgcgcaatgcaggcacaaattaccgccgaacgcgaaaaacgcgcccgtattgccgaatccgaaggccgtaaaatcgaacaaatcaaccttgccagtggtcagcgtgaagccgaaatccaacaatccgaaggcgaggctcaggctgcggtcaatgcgtccaatgccgagaaaatcgcccgcatcaaccgcgccaaaggcgaagcggaatccctgcgccttgttgccgaagccaatgccgaagccatccgtcaaattgccgccgcccttcaaacccaaggcggggcggatgcggtcaatctgaagattgcggaacaatacgtagccgcgttcaacaatcttgccaaagaaagcaatacgctgattatgcccgccaatgttgccgacatcggcagcctgatttctgccggcatgaaaattatcgacagcagcaaaaccgccaaataaggcagtcgtataacctgcaggactagtcggccgtacctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtgggccctttcgtctcgcgcgtttcggtgatagcttaagtgcggtcatcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaaca

SEQ ID NO: 55 - DNA, 7229 bp, synthetic construct
  misc_feature 1..7229: note = pFP12-GNA1220 beta/alpha/beta plasmid
  source 1..7229: mol_type = other DNA
ctgcaggccgtctgaaccgctctaaccgctttttctcggcttaatttttctgtctctgttataaaattgctattcatcttgttcttcttcaaaaaaaagttaagtaaaatacctacctaaatttttactagttcgcaatctacgagcttataacctcgttttttcaattcatttaaaaaatcagattttgagcctaattttgatctattgctatcgttacccgctagaaatacccagtaattacgcaaatcttcattggtaactttcgtaatatcggtgtaatgatcttcgagtatttttaagcaatctctagcccataaaccgtactcgtgattgctcatcttagggttttgcttatcgagtttgacgaacttcccatacttgtttttatgtggaaatactggccgttttgcaacttcttcaattttttgagctgttcgttttttactaccaatcacaaaatttaaagagtgaatagtacgcccacgcttgatttgttcaacctcaacgactaaatcagatttctcgttaatctcagttattgcaggttccaaaacacgttgatttaatgaattaaatctagggtatttattttcaacctgaagccattcttttagtttttctactgtaatttcacgactaccaacagagcgatattgtgtaattagctcataaattcgaattgaatgtacactgttgaaataagcgatatgtttgagttgatattgcgtgaattgccctttaagttgcgttaggtatggcataacttcatcagtcattgcaattctaaaacgcccctctttcttgaaatatgttctagaggaaacccaacgaaattcagttacacggtctttatcttcagttttaacacttcggtcgtaaatccgttttatagccgcctgaatttgcttataggcgttatcttggcttatttctggaaactcacggacaaaatcagccaccgtaaaatcaaaaatcttttgattagatttcggatccatagtcccgatagttaaagctaaaattctgatttcatcaatactcaatcggtaattggcttcaataaggctattagcctttacaacaactaaatcatttggcataagacaacaaatttcctgtttaaaacaacaagcaaaatatacctgttgtttatatataaaacaacaagtattttcttaaaagttgtctataacaggaaatttgttgtcgtataacaggaaatttgttgtcttataacaggaaatttgttgtcgtataacaggaaatttgttgtcgtataagtttgtaacttattgattttactggttttaaaaacgccgaaaacaagtaaaaaacaaaaatataaaaatatagggactttcgtcccttttttgggctttcagccctaattttttcttttttcaggattaaaaattacaaaacccttacagagcaagtaaacttgtttgcttgttctgcaagggttcagcaaccgtagccgtcaggcgtagggcggtagcctataaaagccatttaattttatctttaaacttccttttaaatgctttgagtgggtgtcttttatcgtactcatcaatccttttttgcattctttcgtttgctttgtgatcggcaaattttgaataagatttttccatctcatctaacattctatcaatccgttttttatgttgccatttcaggtaaacataaacacttatagcaattaaagacaatatcaatacattgtaaaaaatgattgttacaatttcgctcacagttattttttacctttttcaatttcttcattgataaatgcactcaattcatcaaatttcttgtcatcattgataaatttacgcaacttagggaagtttctatctacatctaaaagagggctatttattacttcatttagccaaaaagcccctaataaaaccttgtaatgcgtagctttcttacgcttttctgcttgttcttttgacttaatcgcacgaattttcgctttgatttcgtcctgcttgcgttgtaaatctgcttgttgctgttccaatcttgtaagtttttcgcttgccatactagcccctttatatagttagaaattatcgttattttattcagtaggtgctaggcttgcaagtgttctgttcattacgttaaaataacgtaatgcccacttatcagtttctcttcgagaaactggtgggcaagcgtaccgcttgaccgtttcgcaatactcaacactatggcaatctatcatttaaacgttcgctattgcagtaaaagcaaagggcaatcagctcaagccaaaaacgactacatcaaccgcaatgataaatattcaaagcggttagatgatttacagttttcaggctatggtaatatgccaaaatttgccgaagataatccgcaagaattttggcgattgtcagatatttacgagcgagctaatgcccgagtttgtactgaaattgaatttgctttacctagagaattaaccctagaacaacagcaaaaattagtaagttcgtttatagaaaatacggttgatagcggtagcaataaactaccctactctttcgctatccataccgataaaaataatcataatccccattgtcatttgatattttcagaacgccaacttgacggcatagaccgtacagccgagcagttttttaaacgtgctaatactaaatccccagaaaagggcggagcgatgaaaacggcagattttcgagatcgtgagtttatccaatctgtccgaaaaacgtggagagagcaagctaatcaagccttagagcaatacggatatgccgcacgaattgacgaacgtagctacaaggaacaaggcatagagcaagccccaagagcaagaattgacagggtaacgtggcaagaattgaaccgattagagcaagaagaacgccaaatcgtgcaagagcttgcacttaaaggacaagaaattaacaaagaaaaatcctacttgcagaaaatcgaagaaaaacaggctcaaggaatgggcaaatatgaatccaaattcgcagctgcgttttctaaattatcggaaagtgccctaaaacacgatttaagcaacgaaaaagaaaaagacagtaaaatacacactcaagaagaaaaagtgcctcaaaatcgcattcaggggctttctcaagcagattttgatcagtttttaattgatgaatggctacctcaaatagaaaaatacgttaaagcccaagaaaagcgggacggaatggaagtagagatcacgcaatacgacaaggatttacagcgtattcagggagactataacaagctcacagataaaaatcagggttttctcggtttatgggaaactaaagagcaaaaagcaaagaaaaaagagcttgaagatgaatacaaacatacagcagagcaacggaacgctaaaagccaagaattagccgagtatagccaaaaaataaaagcatacgaacagaaaacgctagagccaatcaacgagaagattgccaaatatcaagctgacaaccctgaaataaaaatgcggagcttaggatttgtgaaaaaaattaaggctcgaggggcatataaagcggctcaagagcgaatggagcgagaaaaacagcaccaacaggaaaaacaacagagacatttagagcgagagagtggtttgagcttgtagctaacgccctacgcctacggcttcggttgttcaacccttaaagaactcgcaacaagttgcaaattctttaagggttcgcaataaaaacaaccgctaaacatttctgcccagcggttgaaaatttacctattcaccattacaatgatcaagcaggaaatttttttgattgccgtaaatgtccgtatatctagttgaggcacaacccgccaaagtcattgccccaaccagaacggcgaaagctaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacgggaaacagctatgaccatgattacgccaagcttcctcagcgcatgcatccgggcagcagcgcatcggctcgcacgaggtctgcgcttgaattgtgttgtagaaacacaacgtttttgaaaaaataagctattgttttatatcaaaatataatcatttttaaaataaaggttgcggcatttatcagatatttgttctgaaatgaagacgtatcgggtgtttgcccgatgtttttaggtttttatcaaatttacaaaaggaagcccatatgatggtgaaccgaactgccttctgcctttctctgaccgccgccctgattctgaccgcctgcagcagcggagggggtggtgaacaaatcaaccttgccagtggtcagcgtgaagccgaaatccaacaatccgaaggcgaggctcaggctgcggtcaatgcgtccaatgccgagaaaatcgcccgcatcaaccgcgccaaaggcgaagcggaatccctgcgccttgttgccgaagccaatgccgaagccatccgtcaaattgccgccgcccttcaaacccaaggcggggcggatgcggtcaatctgaagattgcggaacaatacgtagccgcgttcaacaatcttgccaaagaaagcaatacgctgattatgcccgccaatgttgccgacatcggcagcctgatttctgccggcatgaaaattatcgacagcagcaaaaccgccaaataacctgcaggactagtcggccgtacctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtgggccctttcgtctcgcgcgtttcggtgatagcttaagtgcggtcatcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaaca

SEQ ID NO: 56 - DNA, 8066 bp, synthetic construct; identical to SEQ ID NO: 39
  misc_feature 1..8066: note = pFP12-NHba plasmid
  source 1..8066: mol_type = other DNA

(19) United States
(12) Patent Application Publication
(10) Pub. No.: US 2023/0256072 A1
(43) Pub. Date: Aug. 17, 2023

(54) COMPOSITIONS AND METHODS FOR VACCINATION AGAINST NEISSERIA GONORRHOEAE

(71) Applicant: Omvax, Inc., San Francisco, CA (US)

(72) Inventors: Gregory MOE, Oakland, CA (US); Serena GIUNTINI, Walnut Creek, CA (US)

(21) Appl. No.: 18/305,164

(22) Filed: Apr. 21, 2023

Related U.S. Application Data
(63) Continuation of application No. PCT/US2021/056249, filed on Oct. 22, 2021.
(60) Provisional application No. 63/104,819, filed on Oct. 23, 2020.

(51) Int. Cl. A61K 39/095; A61K 41/00; C12N 9/10; C12N 15/74; C12N 1/36

(52) U.S. Cl. CPC A61K 39/095; A61K 41/0047; C12N 9/1025; C12N 15/74; C12N 1/36; A61K 2039/523

(57) ABSTRACT

The disclosure provides compositions, and methods of use thereof, for vaccines for treatment of gonococcal and/or meningococcal infection, comprising native outer membrane vesicle (NOMV) derived from bacteria containing a gonococcal protein that is a lipoprotein or is modified to be a lipoprotein. Also provided are meningococcal strains containing a gene encoding a gonococcal protein that is a lipoprotein or is modified to be a lipoprotein.

### CROSS REFERENCE TO RELATED APPLICATIONS

This application is a bypass continuation of International Application No. PCT/US2021/056249, filed Oct. 22, 2021, which claims the benefit of U.S. Provisional Patent Application No. 63/104,819, filed Oct. 23, 2020, the disclosures of each are hereby incorporated by reference as if written herein in their entireties.

### STATEMENT REGARDING FEDERALLY SPONSORED RESEARCH

This invention was made with government support under grant number R01AI046464, awarded by the National Institutes of Health, National Institute of Allergy and Infectious Diseases. The government has certain rights in the invention.

### INCORPORATION OF SEQUENCE LISTING

The sequence listing that is contained in the file named “OMV0002-201BC1-US,” which is 241 kilobytes as measured in Microsoft Windows operating system and was created on Apr. 20, 2023, is filed electronically herewith and incorporated herein by reference.

### FIELD OF THE DISCLOSURE

The present disclosure relates to recombinant bacteria and vaccines derived from bacterial outer-membrane vesicles.

### BACKGROUND

Neisseria gonorrhoeae (Ng) is an obligate human bacterial pathogen that most commonly colonizes the mucosal surfaces of the reproductive tract including the cervix, uterus, and fallopian tubes of women and the urethra of both men and women. However, other tissues including the rectum, nasopharynx and eyes can also harbor gonococci. The bacteria are most commonly transmitted by direct physical contact between individuals in mucosal secretions and possibly within neutrophils. Despite more than 25 years of work, there is no licensed vaccine against Ng, which causes ˜80 million infections annually worldwide and more than 500,000 cases in the U.S. The number of cases of Ng disease in the U.S. has increased by 67% between 2013 and 2018. In women, Ng infections most frequently occur as cervicitis or pelvic inflammatory disease, which can lead to infertility. Only about half of infected women have clinical manifestations to make them aware of infection, which leads to further spread of disease. Infants born to infected mothers can develop ophthalmia neonatorum, which, if untreated, can cause blindness. In men, most Ng infections are manifested as urethritis. Ng also causes pharyngeal and anorectal infections, particularly in men who have sex with men. In some cases, Ng infections can develop into disseminated infections with bacteremia leading to arthritis, endocarditis or meningitis. Multiple antibiotic resistance leaves fewer options for antibiotic therapy of Ng disease and the threat of a universally resistant pathogen. All of these facets emphasize a significant global public health problem and need for an effective Ng vaccine.

Ng infections do not elicit protective immunity, which can result in multiple reinfections. Thus, development of a successful vaccination strategy against Ng requires eliciting greater protective immunity than natural infection. This will depend on increasing the immunogenicity of protective antigen(s) that are poorly immunogenic in natural infection and/or deleting Ng antigens that provide immune shielding. For example, commercial sex workers who develop antibodies to Ng reduction modifiable protein (Rmp) were 3.4-fold more likely to contract Ng infections than sex workers who lacked the antibodies. Antibodies to Rmp and lipooligosaccharide (LOS) variants have been shown to block functional activity mediated by anti-PorB. However, Nm Rmp does not appear to elicit similar blocking antibodies. Therefore, it is advantageous to express Ng antigens in Nm to eliminate blocking Rmp antibodies and knock out genes that result in LOS variants that do not elicit blocking antibodies.

Because of the varied immune suppression mechanisms utilized by Ng, vaccine approaches based on killed bacteria, outer membrane vesicles (OMV), or pili have not been successful. While progress has been made on several Ng recombinant protein antigens, including adhesin complex protein (ACP), methionine binding protein MetQ, and other antigens discovered by proteomic strategies, as well as truncated LOS, none of these approaches is broadly protective and it is likely that novel vaccine approaches are needed to limit the disease burden of this important pathogen.

Vaccine elicited antibodies that can prevent bacterial adherence and colonization of mucosal tissues is critically important for prevention of disease caused by both Nm and Ng. Nm and Ng are obligate human pathogens that use mechanisms for attachment (CEACAM1, CD46), invasion, and immune shielding that specifically interact with human systems. Antibodies elicited by vaccines (e.g., IgA and IgG) are present in secretions enveloping epithelial cells that are in direct contact with Nm and Ng during the earliest stages of infection and can prevent colonization and invasion.

A vaccine that elicits antibodies directed against mechanisms of adherence and immune shielding could protect the individual during the initial stages of infection from more advanced stages of disease and the unvaccinated by limiting transmission between individuals. The most cost effective and widely used vaccines provide both individual and community protection.

OMVax has developed a versatile vaccine platform based on Neisseria meningitidis (Nm) native outer membrane vesicles (NOMV) for presenting protein antigens to the immune system in a native conformation. Native outer membrane vesicles (NOMV) are blebbed naturally from Neisseria meningitidis (Nm) bacteria. Previously, the vaccine strains have been genetically modified to (a) overexpress Factor H binding protein (FHbp), which is normally present in low abundance, (b) express mutant FHbp with low binding to host Factor H to increase antibody responses that block the interactions causing FH binding, and (c) have attenuated endotoxin, enabling use of NOMV without the detergent treatment that is normally used to decrease reactogenicity but also results in removal or alteration of potentially protective antigens. The NOMV-FHbp with penta-acylated lipooligosaccharide (LOS) resulting from knocking out LpxL1 (LpxL1 KO) decreases cytokine responses in human peripheral blood mononuclear cells (PBMC), which were similar to or lower than those elicited by detergent extracted OMV vaccines that had been safely administered to tens of thousands of human subjects. To further enhance the safety of the NOMV-FHbp vaccine, the strains used to prepare the vaccine incorporate additional genetic deletions that eliminate expression of other undesirable antigens including the group B capsular polysaccharide, and derivatives of LOS, which are known to cross-react with human glycans having similar structures.

The immunogenicity of antigens presented in NOMV is greatly increased versus comparable amounts of the recombinant protein alone. However, generation of the most effective antibody responses require a threshold level of expression that has been achieved by using promoters engineered to produce high rates of transcription, inserting multiple copies in the bacterial genome and transformation with a multi-copy plasmid.

Antigens that bind specifically to host proteins, lipids, or glycans may fail to stimulate antibody responses to the surface of the antigen where binding occurs, since the most important epitopes may be masked by host protein binding and therefore not be accessible to immune recognition. Antigens that bind to host molecules are of particular interest for vaccines, since they typically have a critical role in the mechanism of pathogenesis.

Meningococcal OMVs that contain hexa-acylated lipooligosaccharide produce inflammatory responses. Reactogenicity can be reduced by detergent extraction. However, detergent treatments can result in loss of lipoprotein antigens and alterations in protein structure. The Nm strain used to produce NOMV has the lpxL1 locus disrupted resulting in production of penta-versus hexa-acylated LOS, which results in attenuated endotoxin activity.

The NOMV platform also has adjuvant properties that enhance antibody responses. Overall, NOMV-based vaccines elicit higher titers of antibodies with broader reactivity than the corresponding recombinant proteins and may be more tolerable since less protein may be required to provide an effective protective antibody response.

Several of the Nm proteins that are highly conserved also in Ng have been proposed as antigens for a Ng vaccine. For example, GNA1220 (99% identical; also known as NMB1220 and NGO0788) is related to the stomatin-like family of proteins. Individual members of the family are known by several names, depending on the sequence similarity within sub-families. The names include paraslipin or slipin-2, stomatin, prohibitin, flotillin, and HflK/C. Stomatin-like proteins are single pass, oligomeric membrane proteins of ancient origin that have been identified in all three domains of life. Although their functional role is not completely understood in each instance, they mostly localize to membrane domains; and in many cases, they have been shown to modulate ion channel activity. The conserved domain common to these families has also been referred to as the Band 7 domain. Individual proteins of the family may cluster to form membrane microdomains, which may in turn recruit multiprotein complexes. This subgroup of the stomatin-like proteins remains largely uncharacterized. It includes human stomatin-like protein-2, which is upregulated and involved in the progression and development in several types of cancer, including esophageal squamous cell carcinoma, endometrial adenocarcinoma, breast cancer, and glioma. GNA1220 appears to play a role in increasing Ng survival in human serum and is thought to have a key role in surface colonization as a sensor for initiating the transition from non-adherence to adherence. GNA1220 was identified as a promising meningococcal vaccine candidate. Serum bactericidal activity titers elicited by recombinant GNA1220 against Nm were relatively low compared to other proteins identified by genome sequencing and exploration of GNA1220 as a vaccine antigen was later abandoned because it was also difficult to produce as a recombinant protein.

MetQ (also known in Nm as GNA1946 or NMB1946, and in Ng as NGO2139), which is 97% identical between Nm and Ng, has also been identified as a potential Ng vaccine candidate. MetQ is a multifunctional lipoprotein on the bacterial surface that is involved in methionine transport and Ng adhesion to cervical epithelial cells and monocytes. MetQ also is important for Ng survival in human serum. MetQ is expressed constitutively in growth conditions mimicking infection. Recently, it has been reported that recombinant MetQ formulated with CpG nucleotides elicited high serum antibody titers, as well as secretory IgA, in mice, and decreased the time of Ng vaginal colonization in an estrogen-treated female mouse model of gonococcal infection. Those previous studies, while referring to MetQ as a lipoprotein, actually used a recombinant protein produced in E. coli without lipid attached. The present disclosure, on the other hand, uses a recombinant MetQ construct, which is a lipoprotein produced in Nm as described herein. In some embodiments, mutants of MetQ may also be used in accordance with the present disclosure. For example, as described herein, a novel mutant of MetQ useful for the present disclosure may be a naturally occurring mutant of MetQ referred to herein as MetQSM. MetQ and MetQSM may be useful as a vaccine for treatment of gonococcal and/or meningococcal infection, since MetQ is highly conserved between gonococcus and meningococcus, as described herein.

Neisserial heparin binding antigen (NHBA, also known as NGO1220 and GNA2132) is a lipoprotein that binds heparin and chondroitin sulfate. NHBA is highly conserved among gonococcal strains (>93%) but is less homologous to meningococcal NHBA (˜67%-80%). Although the function of NHBA is unknown, gonococcal NHBA appears to have a role in Ng colonization.

Vaccine immunogenicity studies of GNA1220, MetQ, and/or NHBA used purified recombinant proteins not expressed in NOMV. As described herein, protective antibody responses are greatly improved by presentation of GNA1220, MetQ, MetQSM, and/or NHBA, and derivatives thereof, in NOMV, or a mixture of NOMV containing both proteins, require less protein to produce equal or higher antibody titers in mice and identify derivatives of both proteins that may be advantageous for eliciting antibodies that prevent Ng colonization, thus preventing acquisition and transmission of gonococci.

### SUMMARY

Thus, in one aspect, the disclosure provides a pharmaceutical vaccine composition comprising a plurality of bacterial native outer-membrane vesicles (NOMVs) comprising at least one recombinant protein from Neisseria gonorrhoeae, wherein the gonococcal recombinant protein is a lipoprotein or is modified to be a lipoprotein. In one embodiment, the gonococcal recombinant protein is modified by eliminating portions of the protein that are not surface exposed and adding a lipoprotein signal sequence to the remaining C-terminal portion, wherein the gonococcal recombinant protein is displayed on the surface of the bacteria and NOMV are produced by the bacteria as a lipoprotein. In another embodiment, the at least one gonococcal recombinant protein is GNA1220, MetQ, MetQSM, or NHBA, or derivatives or fragments thereof, or combinations thereof. In another aspect, the NOMVs are derived from Neisseria meningitidis. In another embodiment, the meningococcal strain is H44/76.

In another aspect, the disclosure provides a strain of Neisseria meningitidis comprising at least one gene encoding at least one recombinant protein from Neisseria gonorrhoeae, wherein the at least one gonococcal recombinant protein is a lipoprotein or is modified to be a lipoprotein. In one embodiment, the at least one gonococcal recombinant protein is GNA1220, MetQ, MetQSM, or NHBA, or derivatives or fragments thereof, or combinations thereof. In another embodiment, the at least one gonococcal recombinant protein is expressed from a transgene in a plasmid. In another aspect, the at least one gonococcal recombinant protein is expressed from a transgene inserted in the bacterial genome. In another aspect, the meningococcal strain is H44/76. In another embodiment, the meningococcal strain H44/76 does not express porin PorA. In another embodiment, expression of the transgene encoding the at least one gonococcal recombinant protein is driven by a strong promoter sequence that produces high rates of gene transcription in Neisseria meningitidis. In another embodiment, the strong promoter comprises a PorA promoter or a derivative thereof. In another embodiment, the promoter comprises a sequence set forth in FIGS. 2-4. In another embodiment, the transgene encoding the at least one gonococcal recombinant protein is inserted into the lpxL1 locus of the bacterial genome, wherein the insertion disrupts expression of the acyltransferase gene, and wherein the disruption causes the bacteria to produce a lipooligosaccharide that is penta-acylated and not hexa-acylated. In another embodiment, the transgene encoding the at least one gonococcal recombinant protein is inserted into the siaD-galE locus of the bacterial genome, and wherein the insertion disrupts expression of the capsular polysaccharide and sialylation of the lipooligosaccharide host antigens. In another embodiment, the transgene encoding at least one gonococcal recombinant protein is inserted into the siaA locus. In another embodiment, the transgene encoding the at least one gonococcal recombinant protein is inserted into the fhbp locus (Factor H binding protein). In another embodiment, the transgene encoding the at least one gonococcal recombinant protein is inserted into the porA locus.

In some embodiments, the disclosure provides a pharmaceutical vaccine composition comprising a plurality of bacterial native outer-membrane vesicles (NOMVs) comprising at least one recombinant protein from Neisseria gonorrhoeae, wherein the gonococcal recombinant protein is a lipoprotein or is modified to be a lipoprotein.

In some embodiments, the gonococcal recombinant protein is modified by eliminating portions of the protein that are not surface exposed and adding a lipoprotein signal sequence to the remaining C-terminal portion, wherein the gonococcal recombinant protein is displayed on the surface of the bacteria and NOMV are produced by the bacteria as a lipoprotein.

In some embodiments, the at least one gonococcal recombinant protein is GNA1220, MetQ, MetQSM, or NHBA, or derivatives or fragments thereof, or combinations thereof.

In some embodiments, the NOMVs are derived from Neisseria meningitidis.

In some embodiments, the meningococcal strain is H44/76.

In some embodiments, the disclosure provides a strain of Neisseria meningitidis comprising at least one gene encoding at least one recombinant protein from Neisseria gonorrhoeae, wherein the at least one gonococcal recombinant protein is a lipoprotein or is modified to be a lipoprotein.

In some embodiments, the at least one gonococcal recombinant protein is GNA1220, MetQ, MetQSM, or NHBA, or derivatives or fragments thereof, or combinations thereof.

In some embodiments, the at least one gonococcal recombinant protein is expressed from a transgene in a plasmid.

In some embodiments, the at least one gonococcal recombinant protein is expressed from a transgene inserted in the bacterial genome.

In some embodiments, the meningococcal strain is H44/76.

In some embodiments, the meningococcal strain H44/76 does not express porin PorA.

In some embodiments, expression of the transgene encoding the at least one gonococcal recombinant protein is driven by a strong promoter sequence that produces high rates of gene transcription in Neisseria meningitidis.

In some embodiments, the strong promoter comprises a PorA promoter or a derivative thereof.

In some embodiments, the promoter comprises a sequence set forth in FIGS. 2-4.

In some embodiments, the transgene encoding the at least one gonococcal recombinant protein is inserted into the lpxL1 locus of the bacterial genome, wherein the insertion disrupts expression of the acyltransferase gene, and wherein the disruption causes the bacteria to produce a lipooligosaccharide that is penta-acylated and not hexa-acylated.

In some embodiments, the transgene encoding the at least one gonococcal recombinant protein is inserted into the siaD-galE locus of the bacterial genome, and wherein the insertion disrupts expression of the capsular polysaccharide and sialylation of the lipooligosaccharide host antigens.

In some embodiments, the transgene encoding the at least one gonococcal recombinant protein is inserted into the siaA locus.

In some embodiments, the transgene encoding the at least one gonococcal recombinant protein is inserted into the fhbp locus.

In some embodiments, the transgene encoding the at least one gonococcal recombinant protein is inserted into the porA locus.

These and other embodiments of the disclosure are described in detail below.

### BRIEF DESCRIPTION OF THE SEQUENCES

SEQ ID NO:1—Sequence of MetQ protein.

SEQ ID NO:2—DNA Sequence of MetQ.

SEQ ID NO:3—Sequence of MetQSM protein.

SEQ ID NO:4—DNA sequence of MetQSM.

SEQ ID NO:5—Sequence of GNA1220 protein.

SEQ ID NO:6—DNA Sequence of GNA1220.

SEQ ID NO:7—Sequence of GNA1220αβα protein.

SEQ ID NO:8—DNA Sequence of GNA1220_helix-αβα.

SEQ ID NO:9—Sequence of MetQ_neisseria forward primer.

SEQ ID NO:10—Sequence of MetQ_SbfI reverse primer.

SEQ ID NO:11—Sequence of MetQ_neisseria reverse primer.

SEQ ID NO:12—Sequence of MetQ_SpeI reverse primer.

SEQ ID NO:13—Sequence of GNA1220_StuI reverse primer.

SEQ ID NO:14—Sequence of Blue script plasmid (FHbp KO+MetQ).

SEQ ID NO:15—Sequence of MetQ pBS downstream forward primer.

SEQ ID NO:16—Sequence of RBD pBS downstream reverse primer.

SEQ ID NO:17—Sequence of FHbp upstream forward primer.

SEQ ID NO:18—Sequence of FHbp upstream reverse primer.

SEQ ID NO:19—Sequence of pGEM plasmid (Capsule KO+MetQ).

SEQ ID NO:20—Sequence of Capsule KO GalE forward primer.

SEQ ID NO:21—Sequence of Capsule KO upstream MetQ reverse primer.

SEQ ID NO:22—Sequence of Capsule KO Spc downstream forward primer.

SEQ ID NO:23—Sequence of Capsule KO SiaD reverse primer.

SEQ ID NO:24—Sequence of pUC18 plasmid (lpxL1 KO+MetQ).

SEQ ID NO:25—Sequence of Lpxl1 upstream forward primer.

SEQ ID NO:26—Sequence of Lpxl1 upstream reverse primer.

SEQ ID NO:27—Sequence of Lpxl1 downstream reverse primer.

SEQ ID NO:28—Sequence of Blue script plasmid (FHbp KO+GNA1220).

SEQ ID NO:29—Sequence of GNA1220 pBS downstream forward primer.

SEQ ID NO:30—Sequence of pGEM plasmid (Capsule KO+GNA1220).

SEQ ID NO:31—Sequence of Capsule KO upstream GNA1220 reverse primer.

SEQ ID NO:32—Sequence of pUC18 plasmid (lpxL1 KO+GNA1220).

SEQ ID NO:33—Sequence of pFP12-MetQ plasmid.

SEQ ID NO:34—Sequence of pFP12-MetQSM plasmid.

SEQ ID NO:35—Sequence of pFP12-GNA1220 plasmid (shown in FIG. 2).

SEQ ID NO:36—Sequence of pFP12-GNA1220_helix-αβα plasmid.

SEQ ID NO:37—Sequence of NHBA protein.

SEQ ID NO:38—Sequence of pFP12-NHBA plasmid.

SEQ ID NO:39—Sequence of pFP12-NHBA plasmid.

SEQ ID NO:40—Sequence of pBS-FHbpKO-MetQ plasmid (corresponding to FIG. 12).

SEQ ID NO:41—Sequence of pBS-FHbpKO-MetQSM plasmid (corresponding to FIG. 13).

SEQ ID NO:42—Sequence of pBS-FHbpKO-GNA1220 plasmid (corresponding to FIG. 14).

SEQ ID NO:43—Sequence of pBS-FHbpKO-NHba plasmid (corresponding to FIG. 15).

SEQ ID NO:44—Sequence of pUC18-LpxL1KO-MetQ plasmid (corresponding to FIG. 16).

SEQ ID NO:45—Sequence of pUC18-LpxL1KO-MetQSM plasmid (corresponding to FIG. 17).

SEQ ID NO:46—Sequence of pUC18-LpxL1KO-GNA1220 plasmid (corresponding to FIG. 18).

SEQ ID NO:47—Sequence of pUC18-LpxL1KO-NHba plasmid (corresponding to FIG. 19).

SEQ ID NO:48—Sequence of pGEM-SiaD-GalEKO-MetQ plasmid (corresponding to FIG. 20).

SEQ ID NO:49—Sequence of pGEM-SiaD-GalEKO-MetQSM plasmid (corresponding to FIG. 21).

SEQ ID NO:50—Sequence of pGEM-SiaD-GalEKO-GNA1220 plasmid (corresponding to FIG. 22).

SEQ ID NO:51—Sequence of pGEM-SiaD-GalEKO-NHba plasmid (corresponding to FIG. 23).

SEQ ID NO:52—Sequence of pFP12-MetQ plasmid (corresponding to FIG. 24).

SEQ ID NO:53—Sequence of pFP12-MetQSM plasmid (corresponding to FIG. 25).

SEQ ID NO:54—Sequence of pFP12-GNA1220 plasmid (corresponding to FIG. 26).

SEQ ID NO:55—Sequence of pFP12-GNA1220αβα plasmid (corresponding to FIG. 27).

SEQ ID NO:56—Sequence of pFP12-NHba plasmid (corresponding to FIG. 28).

### BRIEF DESCRIPTION OF THE DRAWINGS

FIG. 1 depicts over-expression of MetQ, MetQSM, and NHBA with three chromosomal copies of MetQ or MetQSM inserted into siaD-galE, lpxL1, and fhbp loci (dashed line histogram) and three copies plus the multi-copy plasmid (solid black line histogram) as described in Example 1, compared to the wild-type strain (gray shaded histogram); and NHBA over-expressed in a strain with siaD-galE, lpxL1, and fhbp loci inactivated, plus the multi-copy plasmid with Ng NHBA (dashed line histogram), compared to wild-type NHBA expression (solid line histogram) as measured by flow cytometry with anti-rMetQ or anti-NHBA polyclonal antibodies, respectively.

FIG. 2 depicts the pFP12-GNA1220WT plasmid.

FIG. 3 depicts the pFP12-GNA1220_helix-αβα plasmid.

FIG. 4 depicts the pFP12-MetQWT plasmid.

FIG. 5 depicts results from an ELISA of anti-MetQ polyclonal antisera from mouse (left) and rabbit (right) binding to NOMV containing recombinant MetQ with 1 chromosomal copy (lower line in both), or 3 copies+the pFP12 plasmid with one copy per plasmid (upper line in both). In this experiment, the NOMV coated on the plate was constant at 10 μg/ml and the polyclonal antibodies were serially diluted as indicated in the figure.

FIG. 6 depicts the IgG titers in individual serum from mice immunized with 3 doses of 10 μg, 5 μg, or 2.5 μg of NOMV with over-expressed MetQ or MetQSM compared to mice immunized with 10 μg of recombinant MetQ or aluminum adjuvant (Alum) alone.

FIG. 7 depicts binding of a 1:200 dilution of polyclonal antibodies produced by immunization with recombinant MetQ (rMetQ), rNHBA (solid line in far-right panel) or NOMV containing over-expressed MetQ, MetQSM, GNA1220, or NHBA to gonococcal strains FA1090 and MS11 by flow cytometry.

FIG. 8 depicts serum bactericidal activity (SBA) titers of polyclonal antibodies produced by immunizing mice with 5 μg of NOMV from the triple knockout parent strain or containing over-expressed MetQ, MetQSM, GNA1220, or NHBA, compared to 10 μg of recombinant MetQ (rMetQ) or recombinant NHBA (rNHBA).

FIG. 9 depicts the inhibitory effect of polyclonal antibodies produced by immunizing mice with rMetQ, rNHBA, NOMV-MetQ, NOMV-MetQSM, NOMV-GNA1220, or NOMV-NHBA on colonization by gonococcal strains FA1090 and MS11 grown in two nutritional conditions of ME180 human cervical cells.

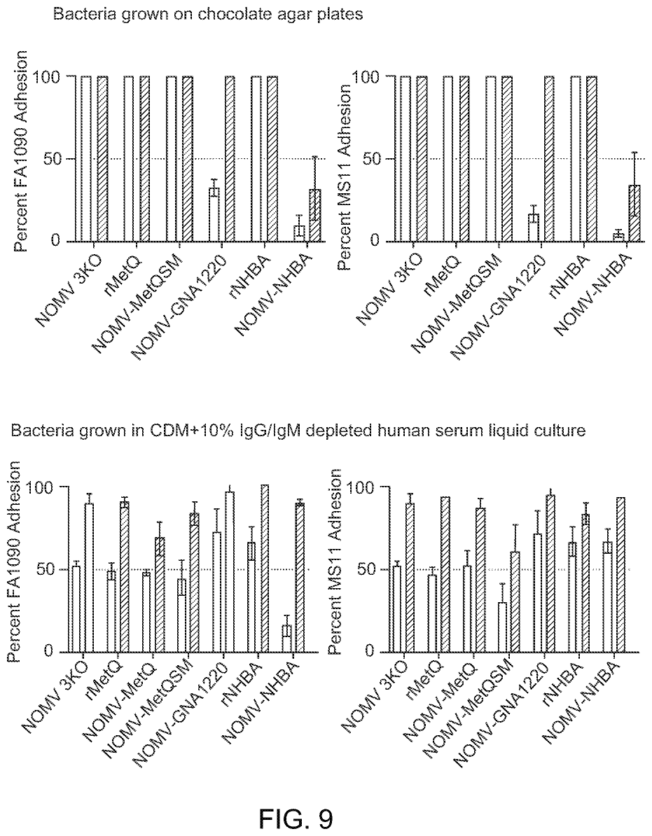

FIG. 10 depicts antibody binding by flow cytometry to Neisseria meningitidis serogroup B strain MD1244 with antiserum (1:200 dilution) from mice immunized with 2 doses of 10 μg of recombinant MetQ or 5 μg of NOMV-MetQ, NOMV-MetQSM, NOMV-GNA1220, or NOMV made from the same strain in which fhbp, siaD-galE, and lpxL1 genes have been knocked out.

FIG. 11 depicts serum bactericidal activity (SBA) of antiserum from mice immunized with 2 doses of 10 μg of recombinant MetQ or 5 μg of NOMV-MetQ, NOMV-MetQSM, NOMV-GNA1220, or NOMV made from the same strain in which fhbp, siaD-galE, and lpxL1 genes have been knocked out.

FIG. 12 depicts the pBS-FHbpKO-MetQ plasmid (corresponding to SEQ ID NO:40).

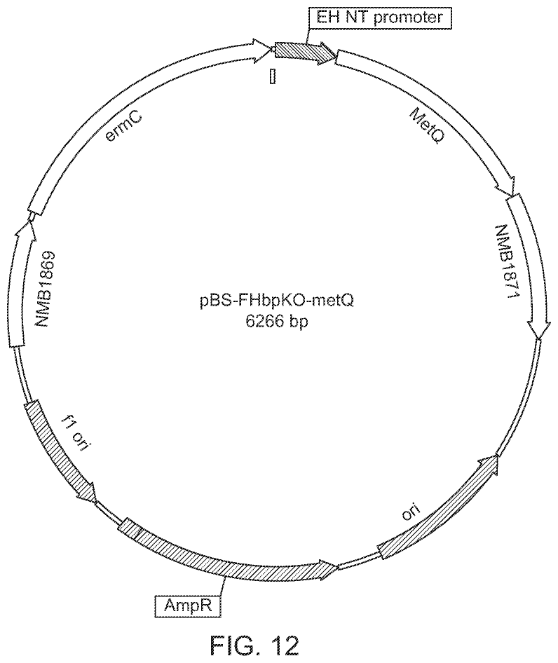

FIG. 13 depicts the pBS-FHbpKO-MetQSM plasmid (corresponding to SEQ ID NO:41).

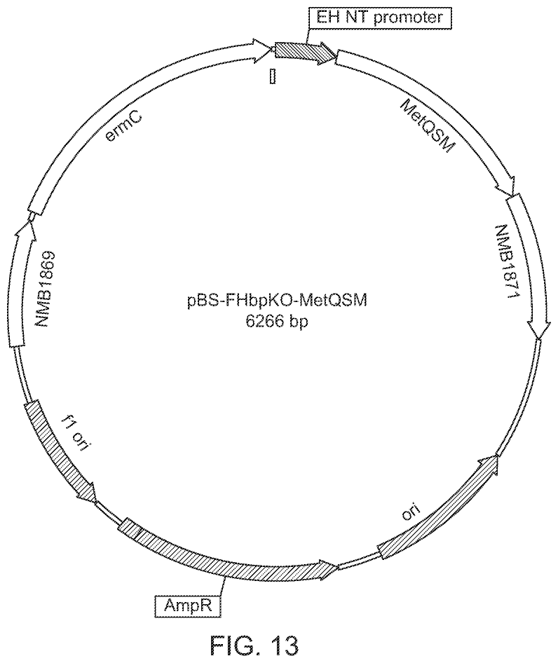

FIG. 14 depicts the pBS-FHbpKO-GNA1220 plasmid (corresponding to SEQ ID NO:42).

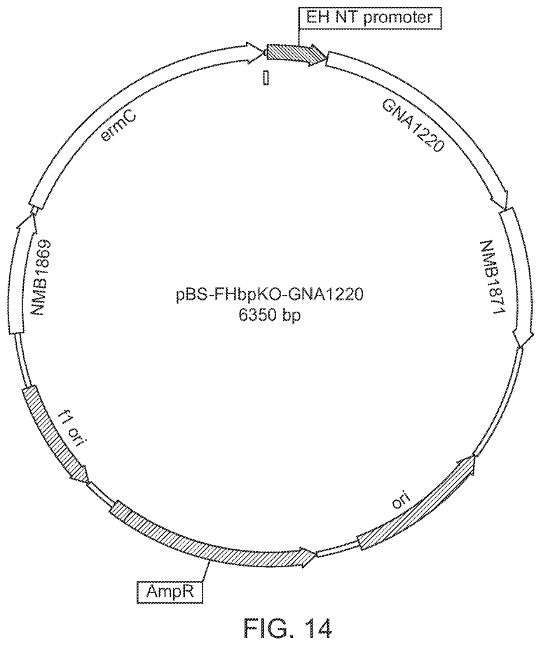

FIG. 15 depicts the pBS-FHbpKO-NHba plasmid (corresponding to SEQ ID NO:43).

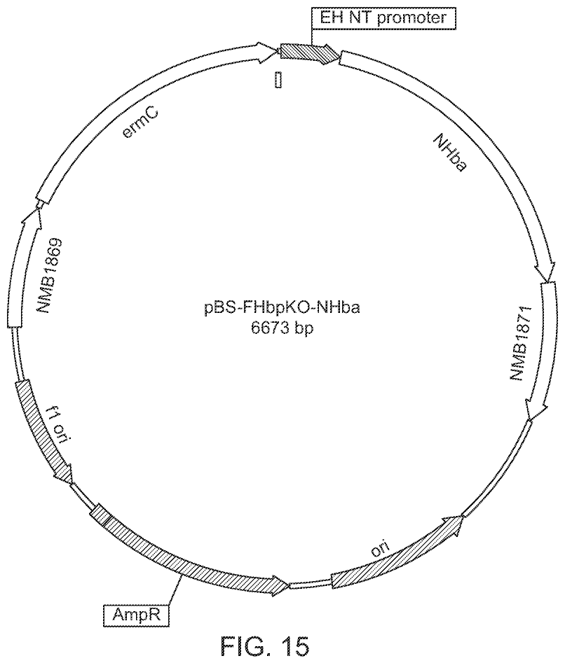

FIG. 16 depicts the pUC18-LpxL1KO-MetQ plasmid (corresponding to SEQ ID NO:44).

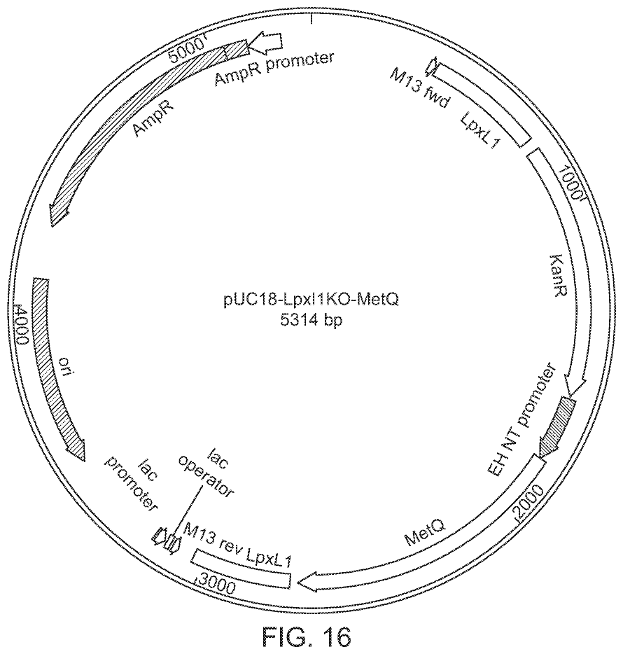

FIG. 17 depicts the pUC18-LpxL1KO-MetQSM plasmid (corresponding to SEQ ID NO:45).

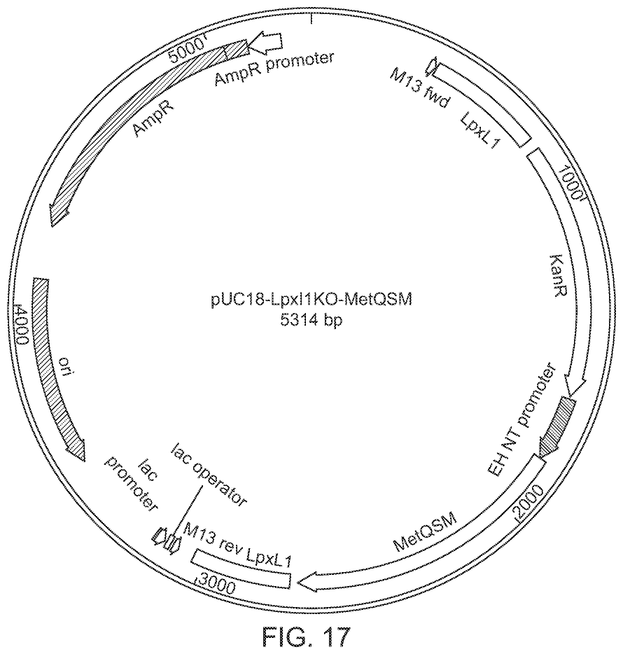

FIG. 18 depicts the pUC18-LpxL1KO-GNA1220 plasmid (corresponding to SEQ ID NO:46).

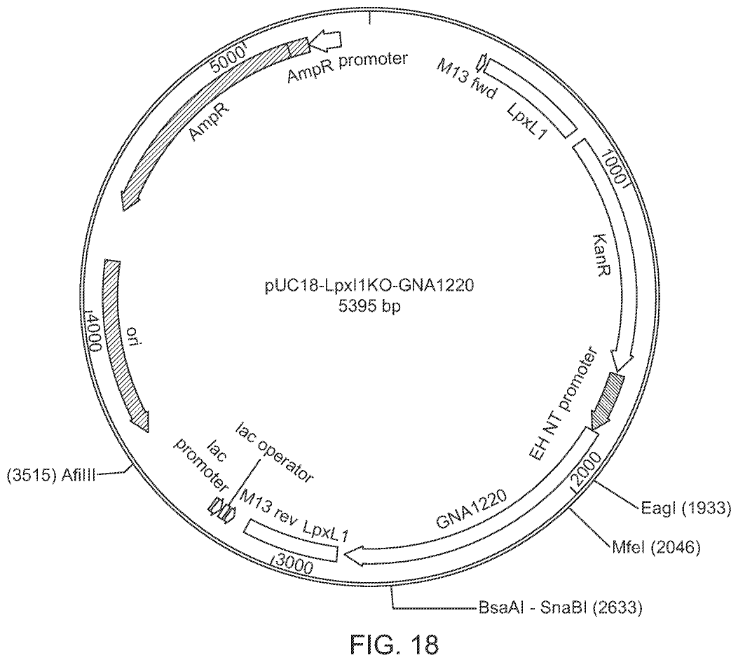

FIG. 19 depicts the pUC18-LpxL1KO-NHba plasmid (corresponding to SEQ ID NO:47).

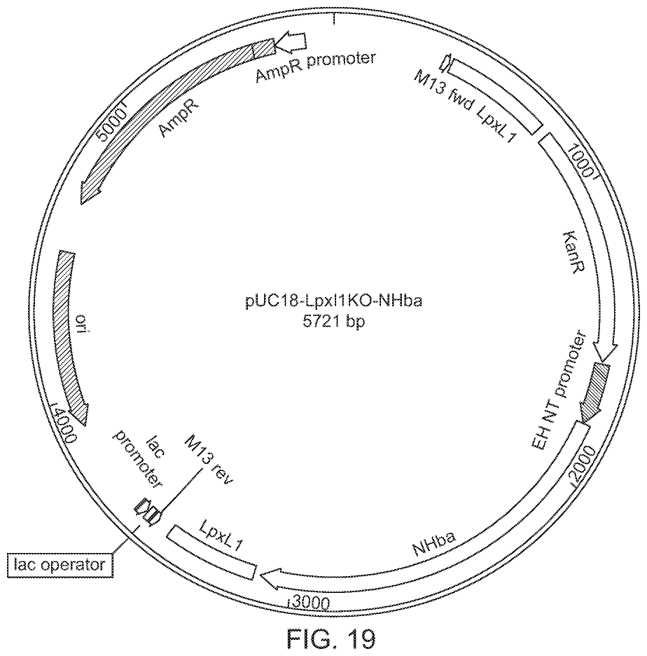

FIG. 20 depicts the pGEM-SiaD-GalEKO-MetQ plasmid (corresponding to SEQ ID NO:48).

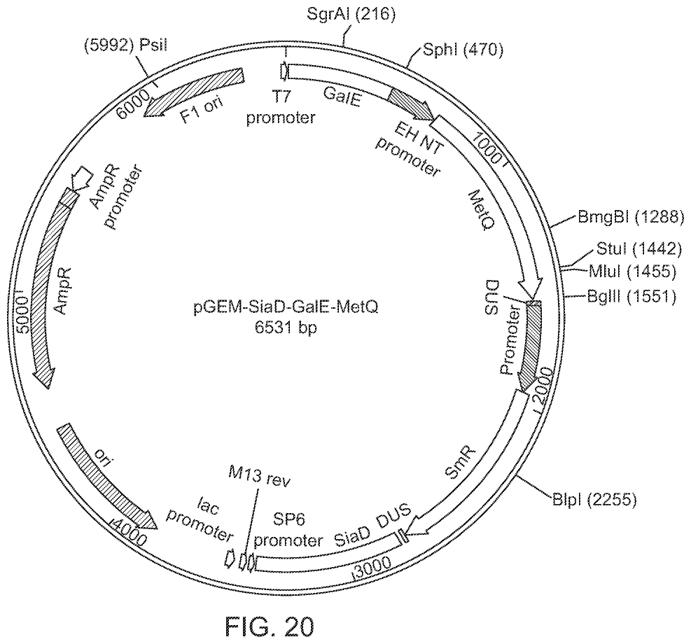

FIG. 21 depicts the pGEM-SiaD-GalEKO-MetQSM plasmid (corresponding to SEQ ID NO:49).

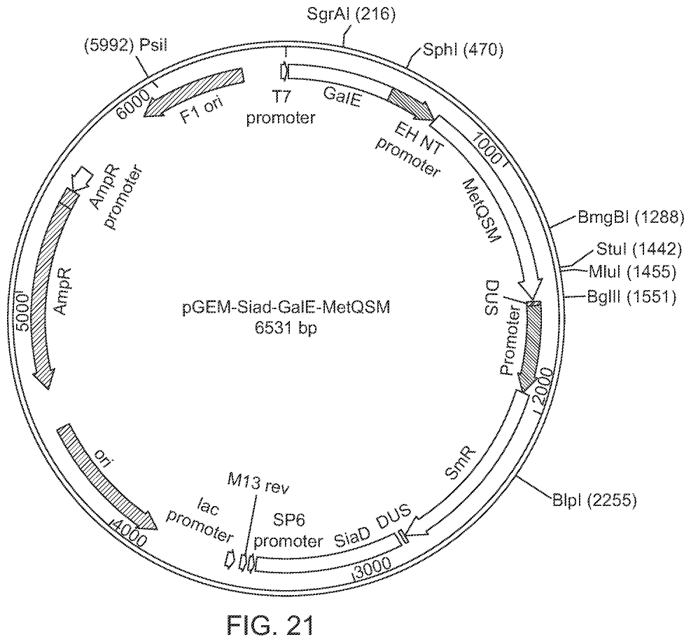

FIG. 22 depicts the pGEM-SiaD-GalEKO-GNA1220 plasmid (corresponding to SEQ ID NO:50).

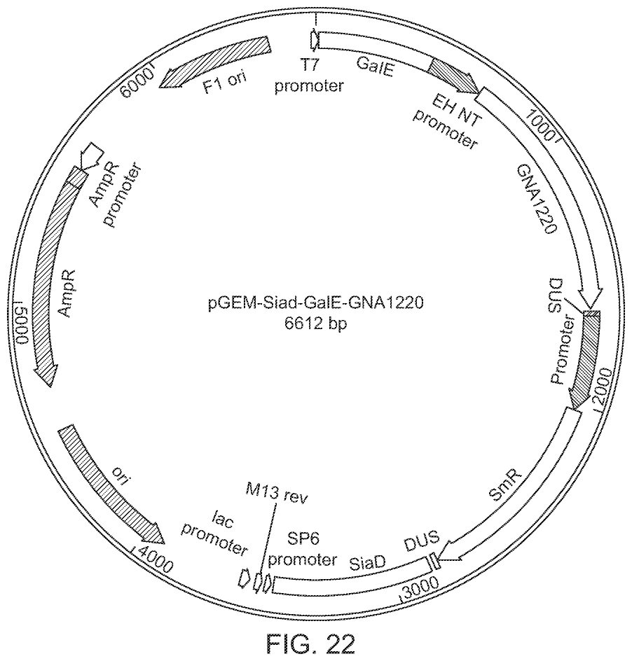

FIG. 23 depicts the pGEM-SiaD-GalEKO-NHba plasmid (corresponding to SEQ ID NO:51).

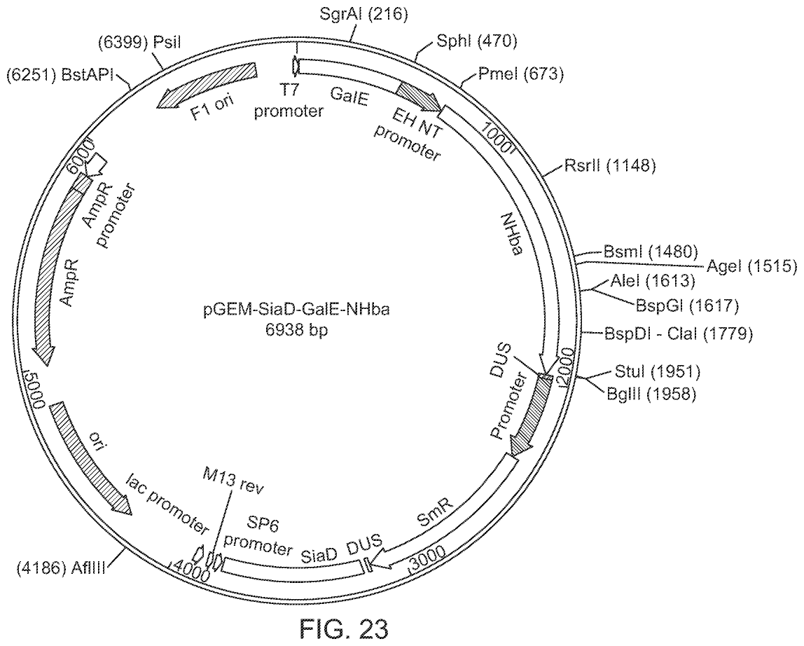

FIG. 24 depicts the pFP12-MetQ plasmid (corresponding to SEQ ID NO:52).

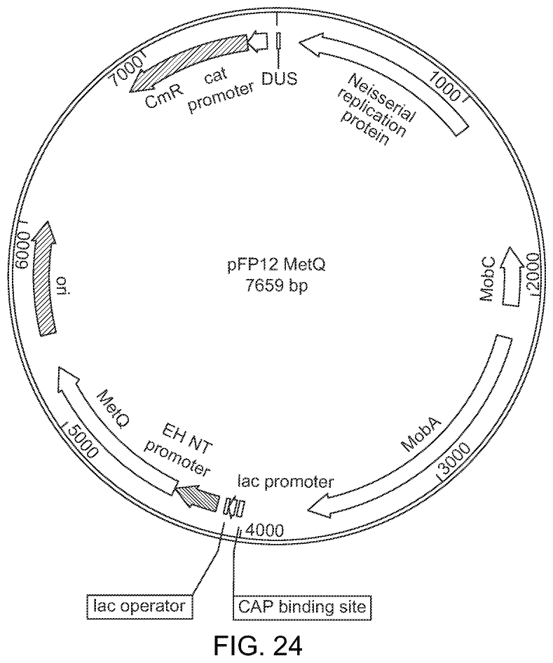

FIG. 25 depicts the pFP12-MetQSM plasmid (corresponding to SEQ ID NO:53).

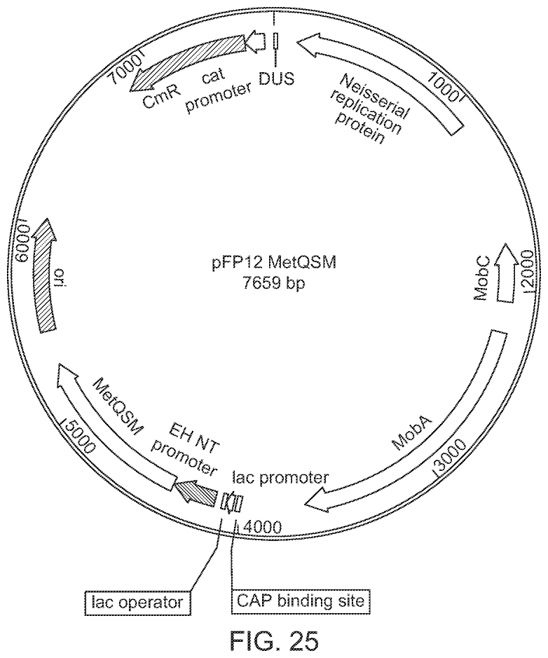

FIG. 26 depicts the pFP12-GNA1220 plasmid (corresponding to SEQ ID NO:54).

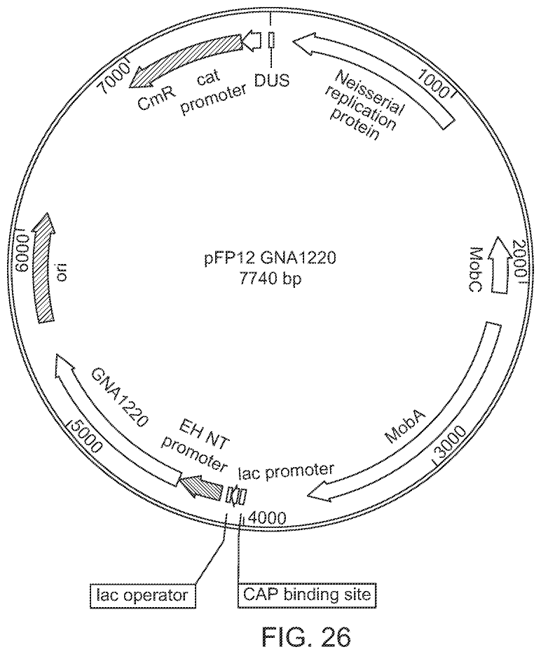

FIG. 27 depicts the pFP12-GNA1220αβα plasmid (corresponding to SEQ ID NO:55).

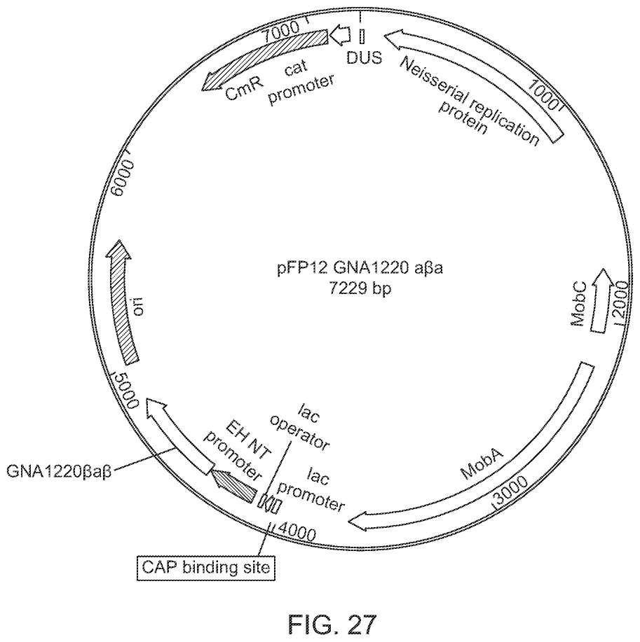

FIG. 28 depicts the pFP12-NHba plasmid (corresponding to SEQ ID NO:56).

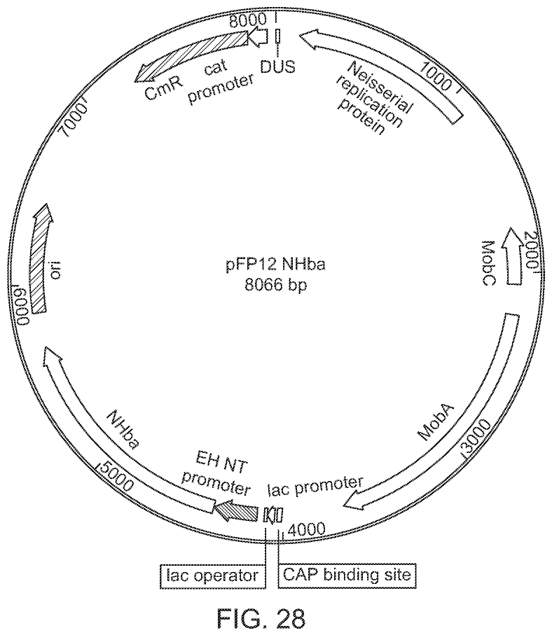

### DETAILED DESCRIPTION

### Overview

The present disclosure describes enhanced protective effects of antibodies against Neisseria gonorrhoeae (Ng) or Neisseria meningitidis (Nm) by (a) overexpression of genes with a novel promoter on a multicopy plasmid and insertion of additional genes in the chromosome to knock out FHbp, capsular polysaccharide, and LOS sialylation, (b) displaying the portions of the proteins on the surface of Neisseria meningitidis (Nm) NOMV, (c) producing the NOMV in a bacterial strain lacking the porin PorA, which is an immunodominant antigen that may, along with capsular polysaccharide, decrease accessibility of the gonococcal proteins to the immune system, and (d) highly overexpressing conserved gonococcal proteins that are normally minor antigens in gonococcus in meningococcal NOMV. For reasons that are poorly understood, but likely depend to some extent on the immune shielding mechanism of Rmp and LOS derivatives described above, the same antigens expressed in gonococcal NOMV are poorly immunogenic and do not elicit antibodies that protect against disease caused by Ng. However, the Rmp and LOS blocking antibodies elicited by Ng NOMV are not elicited by Nm NOMV modified by knocking out the galE locus as described above.

The meningococcal porin protein PorA is one of the most highly expressed proteins in Nm and elicits high titers of anti-PorA antibodies. However, the PorA promoter that drives expression of the gene is phase variable such that insertion or deletion of bases in a polyG tract during replication can result in increased or decreased expression. The Inventors herein have discovered that the region upstream of the PorA gene in Nm contains 6 potential promoters, of which only one contains the polyG tract. Based on this analysis, the PorA promoter was engineered by removing the sequence containing the polyG tract, thus eliminating the potential for phase variation while retaining the ability to drive high levels of transcription. The engineered promoter construct was used to drive expression of gonococcal genes inserted in the chromosome and in the multi-copy plasmid. Promoter gene constructs were inserted in a region encompassing the siaD and galE genes to eliminate the production of capsular polysaccharide and sialylation of LOS, fhbp, and lpxL1, and in the extrachromosomal plasmid. A variant of Nm strain H44/76 lacking PorA expression was selected to increase accessibility of the gonococcal antigens and eliminate potential immunologic competition with an immune-dominant antigen of no value in protection against Ng.

Proteins displayed on the surface of NOMVs are either integral membrane proteins with one or more transmembrane segments or are modified by the attachment of fatty acids to the amino terminal end of the protein producing a lipoprotein where the attached fatty acid acts as an anchor to the membrane. Lipoproteins are initially translated as preprolipoproteins, which possess an amino-terminal signal peptide of around 20 amino acids with typical characteristic features of the signal peptides of secreted proteins. A conserved sequence of the signal peptides, referred to as a lipobox, having consensus amino acid sequences [LVI][ASTVI][GAS]C, is modified through the covalent attachment of a diacylglycerol moiety to the thiol group on the side chain of the indispensable cysteine residue. This modification is catalyzed by the enzyme lipoprotein diacylglyceryl transferase (Lgt), resulting in a prolipoprotein consisting of a diacylglycerol moiety linked by a thioester bond to the protein. After lipidation, lipoprotein signal peptidase (Lsp or SPase II) is responsible for cleaving the signal sequence of the lipidated prolipoprotein and leaves the cysteine of the lipobox as the new amino-terminal residue. In Gram-negative bacteria, such as Neisseria meningitidis, the cleaved prolipoprotein undergoes an additional modification by attachment of an amide-linked acyl group to the N-terminal cysteine residue by lipoprotein N-acyl transferase (Lnt). The diacylglyceryl group and the amino-terminal acyl group are derived from membrane phospholipids and provide tight anchorage of the lipoprotein to the membrane.

Antigens that bind specifically to host proteins, lipids, or glycans may fail to stimulate antibody responses to the surface of the antigen where binding occurs, since they are masked by binding to the respective host protein and therefore not accessible to receptors on antigen-specific B cells. Antigens that bind to host molecules are of particular interest for vaccines, since they typically have a critical role in the mechanism of pathogenesis and are therefore likely to be preserved, despite immune selection pressure.

Neisseria gonorrhoeae (Ng) Protein GNA1220

Structural modelling of GNA1220 has identified 4 structural domains illustrated in the figures below. They include a membrane anchor segment at the N-terminus, the stomatin-like domain, which is known to form ring structures, an extended helical segment, and an alpha-beta-alpha domain (αβα) at the C-terminus. The helical and αβα domains are of particular significance, as they are likely on the external surface of the bacteria and the target of protective antibodies. The Inventors have constructed a lipoprotein variant of GNA1220 that is composed of the lipoprotein signal sequence of FHbp ID9 fused to the helical plus αβα domain of GNA1220, where the helical domain begins just after a possible proteolytic cleavage site (RK) at the C-terminal end of the stomatin-like domain.

Neisseria gonorrhoeae (Ng) Protein MetQ

A naturally occurring mutant of MetQ, referred to herein as MetQSM, prevents methionine binding by stabilizing an open conformation of the protein. Antibodies elicited by a MetQ vaccine antigen locked in the open conformation that bind to the open form of the wild-type protein expressed by Ng may not be able to bind methionine or undergo the conformational change associated with methionine binding resulting in an inability of MetQ to mediate multiple functions associated with resistance to serum and bacterial adhesion.

Neisseria gonorrhoeae (Ng) Protein Neisserial Heparin Binding Antigen (NHBA)

NHBA is a lipoprotein that binds to heparin and chondroitin sulfate and is highly conserved in Ng (97%-100% identity), and may be involved in adhesion of gonococcus to host epithelial cells.

### Native Outer Membrane Vesicles (NOMV) and Vaccines Thereof

In some embodiments, NOMV may be used as a vaccine to treat or prevent gonococcal and/or meningococcal infection in a patient or subject as described herein. NOMV may be administered in a therapeutically effective dose or amount to a patient or subject experiencing symptoms of gonococcal and/or meningococcal infection, or may be administered in a therapeutically effective dose or amount to an asymptomatic patient testing positive for gonococcal and/or meningococcal infection.

The outer membrane of N. meningitidis, which is composed primarily of lipooligosaccharides (LOSs), outer membrane proteins (OMPs), and phospholipids, and is normally very loosely attached to the cell wall. During stationary growth of the bacteria, vesicles or blebs of outer membrane are released into the surrounding medium. These native outer membrane vesicles (NOMV) consist of intact outer membrane, including all of the associated proteins and LOS but lacking the periplasmic and cytoplasmic components. As described herein, the Inventors of the present disclosure have engineered a strain of Neisseria meningitidis (Nm) to express gonococcal proteins, such as GNA1220, MetQ, mutant protein MetQSM, and/or NHBA. As described herein, a NOMV vaccine when administered to a patient in a therapeutically effective or prophylactically effective amount enables both treatment and prevention of gonococcal and/or meningococcal infection, as well as symptoms of infection. Preparation of a NOMV vaccine expressing a gonococcal protein such as GNA1220, MetQ, MetQSM, and/or NHBA is described in the Examples and described in detail herein.

Methods for Treating or Preventing Gonococcal and/or Meningococcal Infection

In some embodiments, the present disclosure provides a method for treatment of gonococcal and/or meningococcal infection comprising administration of a therapeutically effective amount of a NOMV vaccine as described herein to a patient infected with a gonococcal bacterial strain or a meningococcal bacterial strain. In some embodiments, such administration of a NOMV vaccine may be therapeutic and result in amelioration of symptoms associated with gonococcal and/or meningococcal infection in a patient. In other embodiments, such administration of a NOMV vaccine may be prophylactic and result in prevention of infection and development of disease.

A method of the present disclosure may treat or prevent infection of a subject or patient with gonococcal and/or meningococcal infection as described herein. Administration of a composition comprising a NOMV as described herein may be in a clinical setting as described herein, or may be in an alternate setting as deemed appropriate by a clinician or practitioner. Further embodiments for administration of such NOMV vaccines are described herein elsewhere.

In some embodiments, such a composition comprising a NOMV vaccine as described herein may be combined with other therapies or treatments for treatment of gonococcal and/or meningococcal infection in a patient. Any appropriate drug treatment or therapeutic modality may be used as deemed appropriate by a clinician.

Administration of a NOMV vaccine as described herein may reduce the number of days of gonococcal and/or meningococcal symptoms by one or more days, such as reducing symptoms by 1, 2, 3, 4, 5, 6, 7, 8, 9, 10, 11, 12, 13, 14, 15, or the like. Administration of a NOMV vaccine as described herein may be in a single administration or dose, or may be in more than one administration or dose, such as including 2, 3, 4, 5, 6, 7, 8, 9, 10, 11, 12, or more doses. As would be understood by one of skill in the art, some patients or subjects may benefit from more than one administration or treatment with a NOMV vaccine of the present disclosure. Such determination would be made by a clinician or other qualified healthcare personnel.

In other embodiments, symptoms of gonococcal and/or meningococcal infection may be reduced by one week or more, such as including, but not limited to, one week, 2 weeks, 3 weeks, 4 weeks, 5 weeks, 6 weeks, 7 weeks, or 8 weeks, or more. In other embodiments, administration of a NOMV vaccine as described herein may reduce the severity or duration of gonococcal and/or meningococcal infection by 10%, or 20%, or 30%, or 40%, or 50%, or 60%, or 70%, or 80%, or 90%, or 100%.

Unless otherwise specified herein, the methods described herein can be performed in accordance with the procedures exemplified herein or routinely practiced methods well known in the art. See, e.g., Methods in Enzymology, Volume 289: Solid-Phase Peptide Synthesis, J. N. Abelson, M. I. Simon, G. B. Fields (Editors), Academic Press; 1st edition (1997) (ISBN-13: 978-0121821906); U.S. Pat. Nos. 4,965,343, and 5,849,954; Sambrook et al., Molecular Cloning: A Laboratory Manual, Cold Spring Harbor Press, N.Y., (3rd ed., 2000); Brent et al., Current Protocols in Molecular Biology, John Wiley & Sons, Inc. (2003); Davis et al., Basic Methods in Molecular Biology, Elsevier Science Publishing, Inc., New York, USA (1986); or Methods in Enzymology: Guide to Molecular Cloning Techniques Vol. 152, S. L. Berger and A. R. Kimmel Eds., Academic Press Inc., San Diego, USA (1987); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998). The following sections provide additional guidance for practicing the methods of the present disclosure.

### Expression Systems and Vectors Encoding a Recombinant Polypeptide

As detailed herein, the disclosure provides pharmaceutical and therapeutic compositions that can be administered to a mammalian subject in need of long-term in vivo protection against or treatment for gonococcal and/or meningococcal infection. Such compositions typically contain expression systems, e.g., bacterial strains, polynucleotide or polypeptide sequences, expression vectors, or viral vectors that encode or express a recombinant polynucleotide or polypeptide as described herein. In some embodiments, the recombinant polynucleotide or polypeptide that is expressed encodes a gonococcal protein that a lipoprotein or is modified to be a lipoprotein. Compositions of the present disclosure allow optimal in vivo activity or co-expression in a subject or patient (e.g., human or non-human primate) of a recombinant polypeptide as described herein, which provides potent and long-term protection against gonococcal and/or meningococcal infection as described herein.

Optimal expression of a NOMV containing a recombinant polypeptide, such as a gonococcal protein as described herein, can be accomplished via various mechanisms. Such optimal expression may be accomplished using a desired structural design of an expression vector encoding a recombinant polypeptide, or by the use of appropriate regulatory elements in an expression vector. In addition, optimal expression of a recombinant polypeptide of the disclosure in vivo may further be optimized by measurement of cellular levels of the recombinant polypeptide as described herein. Any assays for determination of appropriate levels of the polypeptide may be used as appropriate. Such tests can all be readily carried out via standard assays or protocols well known in the art.

In some embodiments, polynucleotide sequences encoding a recombinant polypeptide, such as a gonococcal protein, as described herein are operably linked to expression control sequences (e.g., promoter sequences) in a bacteria- or virus-based expression vector or expression system described herein. Some examples of a bacterial expression system include, but are not limited to, a meningococcal bacterial strain, such as including, but not limited to, Neisseria meningitidis (N. meningitidis or Nm), Neisseria gonorrhoeae (Ng) or other suitable bacterial strain. In some embodiments, a strain of Nm or Ng bacteria useful for expressing a gonococcal protein may be a strain lacking expression of porn PorA, such as Nm strain H44/76.

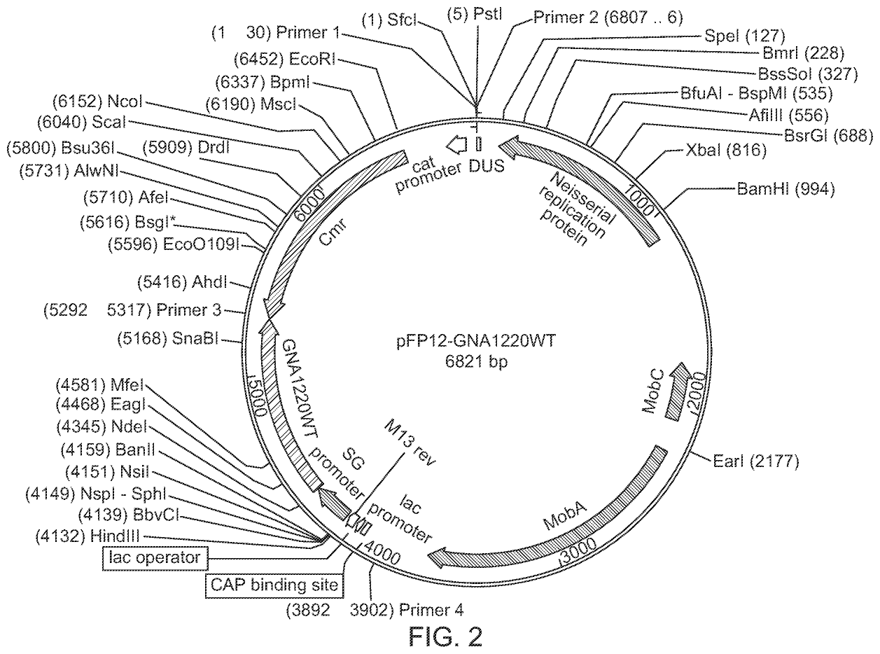

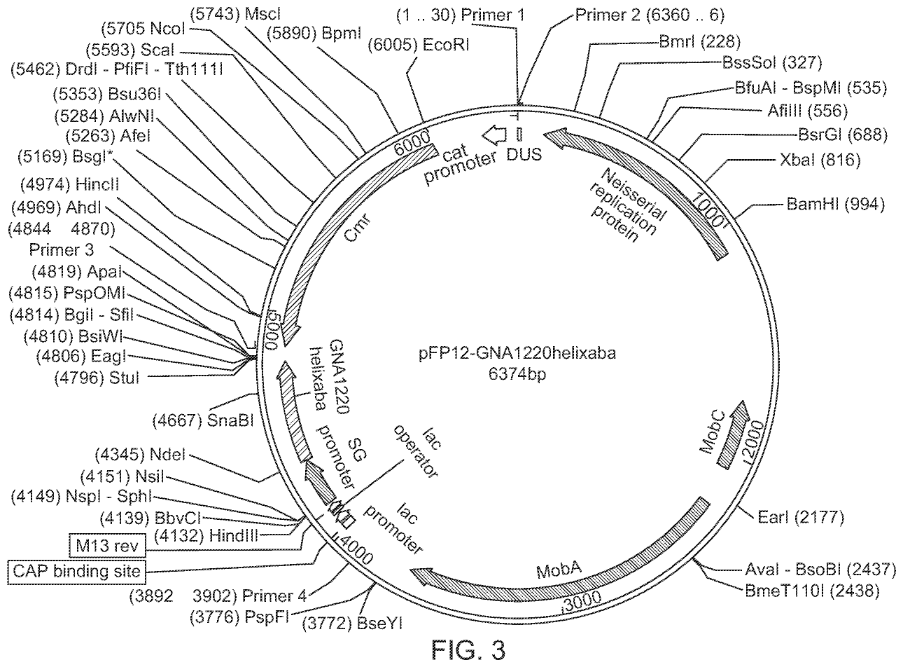

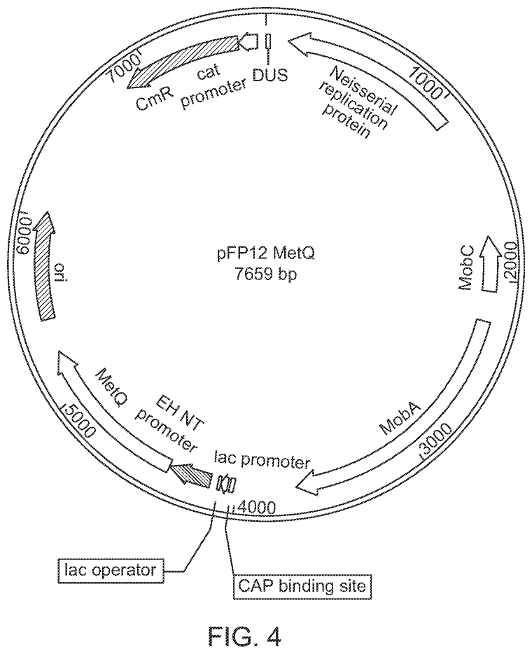

As described herein, Nm may be used to express a gonococcal protein, such as GNA1220, MetQ, MetQSM, or NHBA, or point mutants or portions thereof. In some embodiments, the gonococcal protein is a lipoprotein or is modified to be a lipoprotein. Any useful plasmid known or available in the art may be used to encode and/or express a gonococcal protein in Nm. For example, a vector useful for the present disclosure may be a plasmid. Useful plasmids may include, but are not limited to, any plasmids described herein and capable of carrying and encoding a gonococcal protein as described herein, such as a pFP12-GNA1220WT plasmid (see FIG. 2), a pFP12-GNA1220_helix-αβα (see FIG. 3), a pFP12-MetQWT plasmid (see FIG. 4), a Bluescript plasmid (FHbp KO+MetQ, SEQ ID NO:14), a pGEM Plasmid (Capsule KO+MetQ), SEQ ID NO:19), a pUC18 Plasmid (lpxL1 KO+MetQ, SEQ ID NO:24), a Bluescript plasmid (FHbp KO+GNA1220, SEQ ID NO:28), a pGEM Plasmid (Capsule KO+GNA1220, SEQ ID NO:30), a pUC18 Plasmid (lpxL1 KO+GNA1220, SEQ ID NO:32), a pFP12-MetQ plasmid (SEQ ID NO:33), a pFP12-MetQSM plasmid, SEQ ID NO:34), a pFP12-GNA1220 plasmid (SEQ ID NO:35), a pFP12-GNA1220_helix-αβα plasmid (SEQ ID NO:36), a pFP12-NHBA plasmid (SEQ ID NO:38), a pFP12-NHBA plasmid (SEQ ID NO:39), a pBS-FHbpKO-MetQ plasmid (SEQ ID NO:40), a pBS-FHbpKO-MetQSM plasmid (SEQ ID NO:41), a pBS-FHbpKO-GNA1220 plasmid (SEQ ID NO:42), a pBS-FHbpKO-NHba plasmid (SEQ ID NO:43), a pUC18-LpxL1KO-MetQ plasmid (SEQ ID NO:44), a pUC18-LpxL1KO-MetQSM plasmid (SEQ ID NO:45), a pUC18-LpxL1KO-GNA1220 plasmid (SEQ ID NO:46), a pUC18-LpxL1KO-NHba plasmid (SEQ ID NO:47), a pGEM-SiaD-GalEKO-MetQ plasmid (SEQ ID NO:48), a pGEM-SiaD-GalEKO-MetQSM plasmid (SEQ ID NO:49), a pGEM-SiaD-GalEKO-GNA1220 plasmid (SEQ ID NO:50), a pGEM-SiaD-GalEKO-NHba plasmid (SEQ ID NO:51), a pFP12-MetQ plasmid (SEQ ID NO:52), a pFP12-MetQSM plasmid (SEQ ID NO:53), a pFP12-GNA1220 plasmid (SEQ ID NO:54), a pFP12-GNA1220αβα plasmid (SEQ ID NO:55), or a pFP12-NHba plasmid (SEQ ID NO:56).

Some examples of viral vectors suitable for the disclosure include retrovirus-based vectors, e.g., lentiviruses, adenoviruses, adeno-associated viruses (AAV), and vaccinia vectors. In some embodiments, the structure of the vector may be modified as necessary for optimization of expression or to achieve a desired cellular level, of the recombinant polypeptide, such as including expression controlling elements (e.g., promoter or enhancer sequences). In some embodiments, expression of a gonococcal protein as described herein may be accomplished with the use of a strong promoter that produces high rates of gene transcription in Nm, such as a porin PorA promoter. In some embodiments, the difference between the strength of one promoter relative to another promoter is how strongly it agrees with a “consensus sequence,” that is to say, the sequence of bases that most strongly allows for the binding of the transcription complex to it with high probability. In other embodiments, a promoter may be modified to include, for example, changing the −10 and −35 sequences to match specific sequences from Nm. For example, modification of a promoter sequence as described herein from TATTTG or TACAAA and TAAAGG or TGCCCG to TATAAT and TTGACA, respectively, may be made in order to match, for example, the Sigma70 consensus sequence for Nm.

In some embodiments, such a promoter useful in accordance with the present disclosure may include any promoter sequences set forth herein, or other promoter sequences known and/or available in the art.

In some embodiments, a gonococcal protein and suitable promoter to be expressed in a meningococcal strain, such as Nm, as described herein, can be inserted into a locus of the bacterial genome. Such techniques are known and available in the art. A construct or plasmid as described herein to contain a gonococcal protein and a suitable promoter to achieve high rates of transcription can be inserted into any desired locus in the bacterial genome. Certain loci may be preferable for this, such as a gene conferring a particular trait or gene product to the bacterial cells. For example, as described herein, a gonococcal protein gene and a promoter to ensure high rates of transcription may be inserted into the lpxL1 locus, which disrupts expression of the acyltransferase gene such that the lipooligosaccharide produced is penta-acylated instead of hexa-acylated. In other embodiments, a gonococcal protein gene and a promoter to ensure high rates of transcription may be inserted into the siaD-galE locus (also siaA) to disrupt expression of the capsular polysaccharide and sialylation of lipooligosaccharide (LOS) host antigens. In other embodiments, a gonococcal protein gene and a promoter to ensure high rates of transcription may be inserted into the fhbp locus (Factor H binding protein). In other embodiments, a gonococcal protein gene and a promoter to ensure high rates of transcription may be inserted into the porA locus.

Other promoter sequences well known in the art may be used in accordance with the disclosure. These include, but are not limited to, e.g., CMV promoter, elongation factor-I short (EFS) promoter, chicken-actin (CBA) promoter, EF-la promoter, human desmin (DES) promoter, Mini TK promoter, and human thyroxine binding globulin (TBG) promoter. Additionally, an expression vector of the disclosure may include a number of regulatory elements to achieve optimal expression of the gonococcal protein. For example, a 5′-enhancer element and/or a 5′-WPRE element may be included to elevate expression of the recombinant polypeptide. WPRE is a post-transcriptional response element that has 100% homology with base pairs 1093 to 1684 of the Woodchuck hepatitis B virus (WHYS) genome. When used in the 3′ UTR of a mammalian expression cassette, it can significantly increase mRNA stability and protein yield. As used herein, an “expression cassette” refers to a polynucleotide sequence comprising at least a first polynucleotide sequence capable of initiating transcription of an operably linked second polynucleotide sequence and optionally a transcription termination sequence operably linked to the second polynucleotide sequence. As used herein, an expression cassette may comprise an exogenous nucleic acid encoding a gonococcal protein as described herein operably linked to a promoter as described herein.

By expressing a recombinant polypeptide as described herein in a subject or patient, effective and long-term in vivo protection against and/or treatment of gonococcal and/or meningococcal infection in subjects such as humans. For such a method, a subject may be administered a pharmaceutical composition that contains a therapeutically or pharmaceutically effective amount of a recombinant polypeptide or therapeutic composition or expression system of the disclosure, i.e., encoding a gonococcal protein described herein, such as GNA1220, MetQ, MetQSM, and/or NHBA. In some related embodiments, the disclosure provides therapeutic compositions that contain expression systems for optimally expressing a gonococcal protein as described herein in the subject. The expression systems may be polynucleotide sequences or expression vectors, as well as NOMV, liposomes, or other lipid-containing complexes, and other macromolecular complexes capable of mediating delivery of a polynucleotide sequence to a host cell or subject. Various expression vectors or systems can be employed for expressing a recombinant polypeptide of the disclosure upon administration to a subject. In some embodiments, the expression vectors or expression systems may be based on bacterial vectors. In some embodiments, the expression vectors or expression systems may be based on viral vectors. In some other embodiments, the expression systems are comprised of polynucleotide sequences harboring coding sequences for a recombinant polypeptide as described herein, including deoxyribonucleic acid and ribonucleic acid sequences. In some embodiments, the expression vectors or systems are administered to subjects in the form of a recombinant bacterial strain expressing a gonococcal protein or NOMV vaccine thereof as described herein. The NOMV may be isolated and purified prior to administration to a patient or subject according to methods known in the art. In some embodiments, the expression vectors or systems are administered to subjects in the form of a recombinant virus. For example, the recombinant virus can be a recombinant adeno-associated virus (AAV), e.g., a self-complementary adeno-associated virus (scAAV) vector. Such viral delivery methods allow safe, unobtrusive, and sustained expression of high levels of therapeutics as described herein.

As described above, when using the therapeutic compositions of the disclosure for preventing or treating gonococcal infection in a subject, expression levels of the recombinant polypeptide may be examined during the treatment process. In some embodiments, the administered recombinant polypeptides or compositions result in expression of the recombinant polypeptide in the subject in an amount that is sufficient to reduce the number of bacteria detectable in the subject by at least 2-, 3-, 4-, 5-, 6-, 7-, 8-, 9-, 10-, 15-, 20-, 25-, 30-, 35-, 40-, 45-, 50-, 55-, 60-, 65-, 70-, 75-, 80-, 85-, 90-, 95-, 100-, 150-, 200-, 250-, 300-, 350-, 400-, 450-, 500-, 750-, 1000-fold, or more. In some preferred embodiments, treatment of a subject or patient with a NOMV vaccine as described herein to express a gonococcal protein or a therapeutic or pharmaceutical composition of the disclosure for treatment or prevention of gonococcal and/or meningococcal infection results in a reduction of bacteria or bacterial nucleic acid or proteins, to undetectable levels in the blood or plasma of the treated subject.

An expression vector as described herein may contain the coding sequences and other components or functionalities that further modulate gene delivery and/or gene expression, or that otherwise provide beneficial properties. Such other components include, for example, components that influence binding or targeting to cells (including components that mediate cell-type or tissue-specific binding); components that influence uptake of the vector by the cell; components that influence localization of the transferred gene within the cell after uptake (such as agents mediating nuclear localization); and components that influence expression of the gene. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors may be modified to provide such functionalities. Selectable markers can be positive, negative, or bifunctional. Positive selectable markers allow selection for cells carrying the marker, whereas negative selectable markers allow cells carrying the marker to be selectively eliminated. A variety of such marker genes have been described, including bifunctional (i.e., positive/negative) markers (see, e.g., WO 92/08796; and WO 94/28143). Such marker genes can provide an added measure of control that can be advantageous in gene therapy contexts. A large variety of such vectors are known in the art and are generally available. In some embodiments, insertion of a gonococcal protein either alone, or with a suitable promoter to provide high levels of transcription, into a specific bacterial or viral host gene may provide a screenable or selectable characteristic, e.g., one or more of the lpxL1 locus, which disrupts expression of the acyltransferase gene such that the lipooligosaccharide produced is penta-acylated instead of hexa-acylated, or the siaD-galE locus (also siaA) to disrupt expression of the capsular polysaccharide and sialylation of lipooligosaccharide host antigens, or the fhbp locus (Factor H binding protein), or the porA locus.

Expression vectors or systems suitable for the disclosure include, but are not limited to, isolated polynucleotide sequences, e.g., plasmid-based vectors which may be extra-chromosomally maintained, and viral vectors, e.g., recombinant adenovirus, retrovirus, lentivirus, herpesvirus, poxvirus, papilloma virus, or adeno-associated virus, including viral and non-viral vectors which are present in liposomes, e.g., neutral or cationic liposomes, such as DOSPA/DOPE, DOGS/DOPE, or DMRIE/DOPE liposomes, and/or associated with other molecules, such as DNA-anti-DNA antibody-cationic lipid (DOTMA/DOPE) complexes. Exemplary gene viral or bacterial vectors are known in the art and described below. Vectors may be administered via any route including, but not limited to, intramuscular, buccal, rectal, intracoronary, intravenous, intranasal, trans-vaginal, subcutaneous, intra-arterial, intra-articular, intraperitoneal, parenteral, and transfer to cells may be enhanced using electroporation and/or iontophoresis.

In some embodiments, primers useful for construction of a plasmid as described herein may include any primer described herein. One of skill in the art will understand that other primers or vectors may be used without deviation from the scope of the present disclosure. Some examples of primers useful as described herein are as follows:

Primers useful for construction of pUC18 Lpxl1 and pBS FHbp plasmids:

MetQ WT and N238A mutant:
MetQ_neisseria forward primer,
(SEQ ID NO: 9)
5′-atacaattgCCTCAGCGCATGCATC-3′
MetQ_SbfI reverse primer,
(SEQ ID NO: 10)
5′-tatCCTGCAGGTTATACGACTGCCTTATTTG-3′.
GNA1220:
MetQ_neisseria forward primer,
(SEQ ID NO: 9)
5′-atacaattgCCTCAGCGCATGCATC-3′
GNA1220_SbfI reverse primer,
(SEQ ID NO: 10)
5′-tatCCTGCAGGTTATACGACTGCCTTATTTG-3′.
Primers useful for construction of pGEM SiaD/
GalE plasmid:
MetQ WT, N238A mutant, and GNA1220:
MetQ_neisseria forward primer,
(SEQ ID NO: 9)
5′-atacaattgCCTCAGCGCATGCATC-3′
MetQ_neisseria reverse primer,
(SEQ ID NO: 11)
5′-tattctagaTTATACGACTGCCTTATTTGGC-3′.
MetQ WT and N238A mutant:
MetQ_neisseria forward primer,
(SEQ ID NO: 9)
5′-atacaattgCCTCAGCGCATGCATC-3′
MetQ_SpeI reverse primer,
(SEQ ID NO: 12)
5′-tatACTAGTTTATACGACTGCCTTATTTGGCTG-3′.
Primers useful for construction of pFP12 plasmid:
GNA1220:
MetQ_neisseria forward primer,
(SEQ ID NO: 9)
5′-atacaattgCCTCAGCGCATGCATC-3′
GNA1220_StuI reverse primer,
(SEQ ID NO: 13)
5′-tatAGGCCTTATACGACTGCCTTATTTGGC-3′.

In some embodiments, specific primers may be useful for confirming the presence or absence of genes in Neisseria, for example, the MetQ pBS downstream forward primer (SEQ ID NO:15) and RBD pBS downstream reverse primer (SEQ ID NO:16), which produce a fragment of 800 bp.

In other embodiments, FHbp upstream forward primer (SEQ ID NO:17) and upstream reverse primer (SEQ ID NO:18) may be used, which produce a fragment of 800 bp. In some embodiments, these primers may be used for RBD, as well.

In some embodiments, an upstream 900-bp fragment may be produced with Capsule KO GalE Forward primer (SEQ ID NO:20) and Capsule KO upstream metQ reverse primer (SEQ ID NO:21).

In some embodiments, a downstream 850-bp fragment may be produced with Capsule KO Spc downstream forward primer (SEQ ID NO:22) and Capsule KO SiaD reverse primer (SEQ ID NO:23).

In some embodiments, an approximately 770-bp fragment may be produced with Lpxl1 upstream forward primer (SEQ ID NO:25) and Lpxl1 upstream reverse primer (SEQ ID NO:26).

In some embodiments, metQ pBS downstream forward primer (SEQ ID NO:15) may be used to detect MetQ in the FHbp locus, along with Lpxl1 downstream reverse primer (SEQ ID NO:27).

In some embodiments, an 800-bp fragment may be produced with GNA1220 pBS downstream forward primer (SEQ ID NO:29) and RBD pBS downstream reverse primer (SEQ ID NO:16).

In some embodiments, an 800-bp fragment may be produced with FHbp upstream forward (SEQ ID NO:17) and FHbp upstream reverse (SEQ ID NO:18). In some embodiments, these primers may also be used for RBD.

In some embodiments, Capsule KO GalE Forward primer (SEQ ID NO:20) and Capsule KO upstream GNA1220 reverse primer (SEQ ID NO:31) may be used together.

In some embodiments, a downstream 850-bp fragment may be produced with Capsule KO Spc downstream forward primer (SEQ ID NO:22) and Capsule KO SiaD reverse primer (SEQ ID NO:23).

In some embodiments, an approximately 770-bp fragment may be produced with Lpxl1 upstream forward primer (SEQ ID NO:25) and Lpxl1 upstream reverse primer (SEQ ID NO:26).

In some embodiments, a 650-bp fragment may be produced with GNA1220 pBS downstream forward primer (SEQ ID NO:29) and Lpxl1 downstream reverse primer (SEQ ID NO:27). In some embodiments, the GNA1220 downstream forward primer may be used to detect GNA1220 in the FHbp locus.

In some embodiments, a protein sequence useful for the present disclosure may include, but is not limited to, MetQ (SEQ ID NO:1), MetQSM (SEQ ID NO:3), GNA1220 (SEQ ID NOs:5 and 7), and NHBA (SEQ ID NO:37).

In some embodiments, certain plasmid sequences may be useful in accordance with the present disclosure, such as a Bluescript plasmid (FHbp KO+MetQ, SEQ ID NO:14), or a pGEM Plasmid (Capsule KO+MetQ), SEQ ID NO:19), or a pUC18 Plasmid (lpxL1 KO+MetQ, SEQ ID NO:24), or a Bluescript plasmid (FHbp KO+GNA1220, SEQ ID NO:28), or a pGEM Plasmid (Capsule KO+GNA1220, SEQ ID NO:30), or a pUC18 Plasmid (lpxL1 KO+GNA1220, SEQ ID NO:32), or a pFP12-MetQ plasmid (SEQ ID NO:33), or a pFP12-MetQSM plasmid, SEQ ID NO:34), or a pFP12-GNA1220 plasmid (SEQ ID NO:35), or a pFP12-GNA1220_helix-αβα plasmid (SEQ ID NO:36), or a pFP12-NHBA plasmid (SEQ ID NO:38), or a pFP12-NHBA plasmid (SEQ ID NO:39), or a pBS-FHbpKO-MetQ plasmid (SEQ ID NO:40), or a pBS-FHbpKO-MetQSM plasmid (SEQ ID NO:41), or a pBS-FHbpKO-GNA1220 plasmid (SEQ ID NO:42), or a pBS-FHbpKO-NHba plasmid (SEQ ID NO:43), or a pUC18-LpxL1KO-MetQ plasmid (SEQ ID NO:44), or a pUC18-LpxL1KO-MetQSM plasmid (SEQ ID NO:45), or a pUC18-LpxL1KO-GNA1220 plasmid (SEQ ID NO:46), or a pUC18-LpxL1KO-NHba plasmid (SEQ ID NO:47), or a pGEM-SiaD-GalEKO-MetQ plasmid (SEQ ID NO:48), or a pGEM-SiaD-GalEKO-MetQSM plasmid (SEQ ID NO:49), or a pGEM-SiaD-GalEKO-GNA1220 plasmid (SEQ ID NO:50), or a pGEM-SiaD-GalEKO-NHba plasmid (SEQ ID NO:51), or a pFP12-MetQ plasmid (SEQ ID NO:52), or a pFP12-MetQSM plasmid (SEQ ID NO:53), or a pFP12-GNA1220 plasmid (SEQ ID NO:54), or a pFP12-GNA1220αβα plasmid (SEQ ID NO:55), or a pFP12-NHba plasmid (SEQ ID NO:56).

### Pharmaceutical or Therapeutic Compositions for Preventing Bacterial Infection

In some embodiments, the disclosure provides a therapeutic or pharmaceutical composition comprising a NOMV vaccine expressing a gonococcal protein, such as GNA1220, MetQ, and/or NHBA, or mutants thereof, such as MetQSM, as described herein. Vectors are described in detail above and would be known to one of skill in the art.

In some embodiments, a NOMV expressing a gonococcal protein as described herein may be provided as a pharmaceutical or therapeutic composition to be administered to a subject or patient for treatment of gonococcal or meningococcal infection. A composition of the present disclosure may comprise a NOMV expressing a gonococcal protein as described herein in a single unit, or alternatively, in some embodiments a NOMV expressing a gonococcal protein as described herein may comprise a plurality of NOMV. In some embodiments, NOMV may express the full gonococcal protein, or may express a portion of the gonococcal protein sufficient to provide the desired immunological effect.

In some embodiments, a gonococcal protein as described herein may be provided or administered to a subject or patient as NOMV expressing the gonococcal protein. The disclosure provides a NOMV vaccine, pharmaceutical compositions and related methods of using these vaccines, compositions, or expression systems for inhibiting, preventing, or treating gonococcal and/or meningococcal infections. Also provided is a use of the polynucleotides, polypeptides, and expression vectors or systems described herein for the manufacture of a medicament to prevent or treat gonococcal and/or meningococcal infections. The pharmaceutical composition can be either a therapeutic formulation or a prophylactic formulation. Typically, a pharmaceutical composition may contain one or more active ingredients and, optionally, some inactive ingredients. In some embodiments, the active ingredient may be a NOMV vaccine, recombinant polypeptide, an expression vector, or an expression system as described herein. In some other embodiments, the active ingredient may include other antibacterial agents in addition to the expression system of the disclosure. The composition may additionally include one or more pharmaceutically acceptable vehicles and, optionally, other therapeutic ingredients (for example, antibiotics). Various pharmaceutically acceptable additives may also be used in such compositions.

In some embodiments, a NOMV vaccine for treatment of gonococcal and/or meningococcal infection as described herein, along with recombinant bacteria comprising a construct or plasmid encoding a gonococcal protein, and pharmaceutical compositions thereof, as described herein, may be administered in any appropriate dosage to obtain a therapeutic result. As would be understood by one of skill in the art, a dosage of NOMV appropriate for treatment or prevention of gonococcal and/or meningococcal infection or to achieve a particular outcome will vary depending on various factors including, but not limited to, the gene and promoter chosen, the condition, patient-specific parameters, e.g., height, weight, and age, and whether prevention or treatment is to be achieved. A NOMV vaccine of the disclosure may conveniently be provided in the form of formulations suitable for administration, e.g., into the blood stream (e.g., in an intracoronary artery). A suitable administration format may best be determined by a medical practitioner or clinician for each patient individually, according to standard procedures and may include, but is not limited to, intramuscular, buccal, rectal, intracoronary, intravenous, intranasal, trans-vaginal, subcutaneous, intra-arterial, intra-articular, intraperitoneal, parenteral or any other suitable mode of administration known in the art.

A vaccine or pharmaceutical composition of the disclosure may be prepared in accordance with standard procedures well known in the art. See, e.g., Remingtons Pharmaceutical Sciences, 19th Ed., Mack Publishing Company, Easton, Pa., 1995; Sustained and Controlled Release Drug Delivery Systems, J. R. Robinson, ed., Marcel Dekker, Inc., New York, 1978; U.S. Pat. Nos. 4,652,441; 4,917,893; 4,677,191; 4,728,721; and 4,675,189. Pharmaceutical compositions of the disclosure may be readily employed in a variety of therapeutic or prophylactic applications for preventing or treating gonococcal and/or meningococcal infections. For subjects at risk of developing a gonococcal and/or meningococcal infection, a vaccine composition of the disclosure may be administered to provide prophylactic protection against gonococcal and/or meningococcal infection. Depending on the specific subject and conditions, a composition of the disclosure may be administered to a subject or patient by a variety of administration modes known to the person of ordinary skill in the art, for example, intramuscular, subcutaneous, intravenous, intra-arterial, intra-articular, intraperitoneal, or parenteral routes. In some embodiments, a composition as described herein may be administered to a subject in need of such treatment for a time and under conditions sufficient to prevent, inhibit, and/or ameliorate a selected disease or condition or one or more symptom(s) thereof. For therapeutic applications, a composition may contain a therapeutically effective amount of the expression system described herein. For prophylactic applications, a composition as described herein may contain a prophylactically effective amount of an expression system as described herein. The appropriate amount of the expression system (e.g., expression vectors) may be determined based on the specific disease or condition to be treated or prevented, severity, age of the subject, and other personal attributes of the specific subject (e.g., the general state of the subject's health and the robustness of the subject's immune system). Determination of effective dosages may additionally be guided with animal model studies (i.e., primate, canine, or the like), followed by human clinical trials, and by administration protocols that significantly reduce the occurrence or severity of targeted disease symptoms or conditions in the subject.

For prophylactic applications, a NOMV vaccine as described herein may be provided in advance of any symptom, for example in advance of infection. A prophylactic administration of the immunogenic compositions may serve to prevent or ameliorate any subsequent infection. Thus, in some embodiments, a subject to be treated is one who has, or is at risk for developing, a gonococcal and/or meningococcal infection, for example because of exposure or the possibility of exposure to the bacterium. Following administration of a therapeutically effective amount of the disclosed therapeutic compositions, a subject or patient may be monitored for gonococcal and/or meningococcal infection, symptoms associated with gonococcal and/or meningococcal infection, or both.

For therapeutic applications, a composition as described herein may be provided at or after the onset of a symptom of disease or infection, for example after development of a symptom of gonococcal and/or meningococcal infection, or after diagnosis of infection. A composition as described herein may thus be provided prior to the anticipated exposure to a gonococcal bacterial strain or a meningococcal bacterial strain, so as to attenuate the anticipated severity, duration or extent of an infection and/or associated disease symptoms, after exposure or suspected exposure to the bacterium, or after the actual initiation of an infection.

In some embodiments, a NOMV vaccine of the disclosure may be provided in a dosage form containing an amount of NOMV expressing or comprising a gonococcal protein that is effective in one or multiple doses. An effective dose may be any range deemed appropriate by a clinician or practitioner. Administration of a NOMV vaccine with the gonococcal protein, a recombinant bacterial strain expressing a gonococcal protein, or a composition comprising any of these may be in a buffer, such as phosphate-buffered saline, or other appropriate buffer or diluent. The amount of buffer or diluent may vary and would be determined by a clinician or practitioner. For delivery to a cell of plasmid DNA alone, or plasmid DNA in a complex with other macromolecules, the amount of DNA to be administered would be an amount that results in a beneficial effect to the recipient. For example, from 0.0001 to 1 mg or more, e.g., up to 1 g, in individual or divided doses, e.g., from 0.001 to 0.5 mg, or 0.01 to 0.1 mg, of DNA can be administered. For delivery of a recombinant polypeptide, such as the gonococcal protein (e.g., GNA1220, MetQ, MetQSM, and/or NHBA) or derivatives thereof, as described herein, an amount administered would be an amount that results in a beneficial effect to the recipient. For example, from 0.0001 to 100 g or more, e.g., up to 1 g, in individual or divided doses, e.g., from 0.001 to 0.5 g, or 0.01 to 0.1 g, of recombinant polypeptide can be administered. For delivery of a NOMV vaccine as described herein, an amount administered would be an amount that results in a beneficial effect to the recipient, whether therapeutic or prophylactic. Such amounts or volumes would be determined by a clinician or practitioner.

In some embodiments, a composition of the disclosure may be combined with other agents known in the art for treating or preventing gonococcal and/or meningococcal infections. These may include any drug known or available in the art for treating a bacterial infection, e.g., antibodies or other antibacterial agents such as antibacterial compounds or drugs, protease inhibitors, fusion protein inhibitors, or the like. In some embodiments, a composition as described herein for treatment or prevention of gonococcal and/or meningococcal infection may be advantageous in situations where a patient or subject is unresponsive to antibiotic treatment due to an increase in antibiotic resistance in the bacteria. Administration of a composition and one or more known anti-bacterial agent may be either concurrently or sequentially.

As described herein, NOMV-based vaccines elicit higher titers of antibodies with broader reactivity than the corresponding recombinant proteins and may be more tolerable since less protein may be required to provide an effective protective antibody response. Thus, in some embodiments, NOMV may be administered with an adjuvant in order to enhance antibody responses. Suitable adjuvants are known in the art and can include, but are not limited to, aluminum compounds [e.g., amorphous aluminum hydroxyphosphate sulfate (AAHS), aluminum hydroxide, aluminum phosphate, potassium aluminum sulfate (Alum), aluminum hydroxide adjuvant (2% ALHYDROGEL)], cytosine phosphoguanine (CpG) nucleotides (e.g., CpG 1018), AS01, AS04, QS-21, RIBI, MF59, or the like.

### Expression of Nucleic Acids

Polynucleotides useful in the present disclosure can be provided in an expression construct. Expression constructs of the disclosure generally include regulatory elements that are functional in the intended host cell in which the expression construct is to be expressed. Thus, a person of ordinary skill in the art can select regulatory elements for use in, for example, bacterial host cells, yeast host cells, mammalian host cells, and human host cells. Regulatory elements used for expression of nuclear genes include promoters, transcription termination sequences, translation termination sequences, enhancers, and polyadenylation elements. As used herein, the term “expression construct” refers to a combination of nucleic acid sequences that provides for transcription of an operably linked nucleic acid sequence. As used herein, the term “operably linked” refers to a juxtaposition of the components described wherein the components are in a relationship that permits them to function in their intended manner. In general, operably linked components are in contiguous relation.

An expression construct of the disclosure can comprise a promoter sequence operably linked to a polynucleotide sequence encoding a polypeptide of the disclosure. Promoters can be incorporated into a polynucleotide using standard techniques known in the art. Multiple copies of promoters or multiple promoters can be used in an expression construct of the disclosure. In a preferred embodiment, a promoter can be positioned about the same distance from the transcription start site in the expression construct as it is from the transcription start site in its natural genetic environment. Some variation in this distance is permitted without substantial decrease in promoter activity. A transcription start site is typically included in the expression construct.

Nuclear Expression constructs of the disclosure may optionally contain a transcription termination sequence, a translation termination sequence, a sequence encoding a signal peptide, and/or enhancer elements. Transcription termination regions can typically be obtained from the 3′ untranslated region of a eukaryotic or viral gene sequence. Transcription termination sequences can be positioned downstream of a coding sequence to provide for efficient termination. A signal peptide sequence is a short amino acid sequence typically present at the amino terminus of a protein that is responsible for the relocation of an operably linked mature polypeptide to a wide range of post-translational cellular destinations, ranging from a specific organelle compartment to sites of protein action and the extracellular environment. Targeting gene products to an intended cellular and/or extracellular destination through the use of an operably linked signal peptide sequence is contemplated for use with the polypeptides of the disclosure. Classical enhancers are cis-acting elements that increase gene transcription and can also be included in the expression construct. Classical enhancer elements are known in the art, and include, but are not limited to, the cytomegalovirus (CMV) early promoter enhancer element, and the SV40 enhancer element. Intron-mediated enhancer elements that enhance gene expression are also known in the art. These elements must be present within the transcribed region and are orientation dependent.

DNA sequences that direct polyadenylation of mRNA transcribed from the expression construct can also be included in the expression construct, such as an SV40 poly A signal, and include, but are not limited to, an octopine synthase or nopaline synthase signal.

Polynucleotides of the present disclosure can be composed of either RNA or DNA, or hybrids thereof. The present disclosure also encompasses those polynucleotides that are complementary in sequence to the polynucleotides disclosed herein. Polynucleotides and polypeptides of the disclosure can be provided in purified or isolated form.

### Nucleic Acids

Any number of methods well known to those skilled in the art can be used to isolate and manipulate a DNA molecule. For example, as previously described, PCR technology may be used to amplify a particular starting DNA molecule and/or to produce variants of the starting DNA molecule. DNA molecules, or fragments thereof, can also be obtained by any techniques known in the art, including directly synthesizing a fragment by chemical means. Thus, all or a portion of a nucleic acid as described herein may be synthesized.

As used herein, the terms “nucleic acid” and “polynucleotide” refer to a deoxyribonucleotide, ribonucleotide, or a mixed deoxyribonucleotide and ribonucleotide polymer in either single- or double-stranded form, and unless otherwise limited, would encompass known analogs of natural nucleotides that can function in a similar manner as naturally occurring nucleotides. The polynucleotide sequences include the DNA strand sequence that is transcribed into RNA and the strand sequence that is complementary to the DNA strand that is transcribed. The polynucleotide sequences also include both full-length sequences as well as shorter sequences derived from the full-length sequences. The polynucleotide sequence includes both the sense and antisense strands either as individual strands or in the duplex.

### Kits

The disclosure further provides a kit comprising one or more single-use containers comprising a NOMV vaccine as described herein. In some embodiments, a kit of the disclosure may provide a composition comprising a NOMV vaccine for treatment or prevention of gonococcal and/or meningococcal infection as described herein. In other embodiments, a kit as described herein may provide a bacterial strain as described herein, for example, in culture or as a frozen stock combined with, e.g., glycerol. In some embodiments, a kit may provide a pharmaceutical composition comprising a NOMV vaccine or purified preparation of a gonococcal protein, such as GNA1220, MetQ, MetQSM, and/or NHBA, as a polypeptide (e.g., mixed with an adjuvant) as described herein, for administration to a subject or patient. In other embodiments, sterile reagents and/or supplies for administration of a NOMV vaccine, purified gonococcal protein, RNA, vectors, and/or pharmaceutical composition as described herein, may be provided as appropriate. A kit may further comprise reagents for cell transformation and/or transfection, bacterial or viral culture, or the like.

Components provided in a kit of the disclosure may include, for example, any starting materials useful for performing a method as described herein. Such a kit may comprise one or more such reagents or components for use in a variety of assays, including for example, nucleic acid assays, e.g., PCR or RT-PCR assays, luciferase (Luc) assays, cell transformation/transfection, viral/cell culture, blood assays, i.e., complete blood count (CBC), viral titer/viral load assays, antibody assays, viral antigen detection assays, DNA or RNA detection assays, bacterial titer assays, virus neutralization assays, genetic complementation assays, or any assay useful in accordance with the disclosure. Components may be provided in lyophilized, desiccated, or dried form as appropriate, or may be provided in an aqueous solution or other liquid media appropriate for use in accordance with the disclosure.

Kits useful for the present disclosure may also include additional reagents, e.g., buffers, substrates, antibodies, ligands, detection reagents, media components, such as salts including MgCl₂, a polymerase enzyme, deoxyribonucleotides, ribonucleotides, expression vectors, and the like, reagents for DNA isolation, DNA/RNA transfection, or the like, as described herein. Such reagents or components are well known in the art. In some embodiments, one or more adjuvants described herein may be included with a kit of the present disclosure. Where appropriate, reagents included with such a kit may be provided either in the same container or media as a primer pair or multiple primer pairs. In some embodiments, such reagents may be placed in a second or additional distinct container into which an additional composition or reagents may be placed and suitably aliquoted. Alternatively, reagents may be provided in a single container means. A kit of the disclosure may also include packaging components, instructions for use, including storage requirements for individual components as appropriate. Such a kit as described herein may be formulated for use in a clinical setting, such as a hospital, treatment center, or clinical setting, or may be formulated for personal use as appropriate.

### Definitions

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

Unless defined otherwise, all technical and scientific terms used herein have the meaning commonly understood by one of ordinary skill in the art to which the disclosure pertains. Specific terminology of particular importance to the description of the present disclosure is defined below.

As used in this specification and the appended claims, the singular forms “a,” “an,” and “the,” along with similar references used in the context of describing a particular embodiment (especially in the context of certain of the following claims), can be construed to cover both the singular and the plural, unless specifically noted otherwise. Thus, for example, “an active agent” refers not only to a single active agent, but also to a combination of two or more different active agents, “a dosage form” refers to a combination of dosage forms, as well as to a single dosage form, and the like. In some embodiments, the term “or” as used herein, including the claims, is used to mean “and/or” unless explicitly indicated to refer to alternatives only or the alternatives are mutually exclusive.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. In some embodiments, “about” refers to a specified value +/−10%, or 9%, or 8%, or 7%, or 6%, or 5%, or 4%, or 3%, or 2%, or 1%.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and can also cover other unlisted steps. Similarly, any composition or device that “comprises,” “has,” or “includes” one or more features is not limited to possessing only those one or more features and can cover other unlisted features.

As used herein, an “adverse event” refers to any untoward medical occurrence associated with the use of a drug or vaccine as described herein in humans, whether or not considered drug related. An AE or suspected adverse reaction may be considered a “serious adverse event” if it results in any of the following outcomes: death, or immediate risk of death, inpatient hospitalization or prolongation of existing hospitalization, persistent or significant incapacity or substantial disruption of the ability to conduct normal life functions, congenital anomaly/birth defect. An adverse event may also be an important medical event that may not result in death, be life-threatening, or require hospitalization, but may jeopardize the patient or subject and may require medical or surgical intervention to prevent one of the above outcomes. In some embodiments, an adverse event refers to an infusion reaction as a result of administration of a drug or vaccine as described herein.

As used herein, “anaphylaxis” refers to a severe, acute onset allergic reaction that may occur over minutes to several hours. Anaphylaxis may involve the skin, mucosal tissue, or both, and may have one or more symptoms including, but not limited to, generalized hives, pruritus (itching), flushing, swelling of the lips, tongue, throat or uvula, shortness of breath, vomiting, lightheadedness, wheezing, hemodynamic instability, and rash or urticaria. In addition, anaphylaxis may be accompanied by at least one of the following: respiratory compromise (e.g., dyspnea, wheeze-bronchospasm, stridor, reduced peak expiratory flow, hypoxemia), and reduced blood pressure (i.e., systolic blood pressure <90 mm Hg or greater than 30% decrease from that person's baseline) or associated symptoms of end-organ failure (e.g., hypotonia [collapse], syncope, incontinence). Anaphylaxis in accordance with the disclosure is defined by the National Institute of Allergy and Infectious Disease/Food Allergy and Anaphylaxis Network (NIAID/FAAN) clinical criteria for diagnosing anaphylaxis.

As used herein, the terms “antigen” or “immunogen” are used interchangeably to refer to a substance, typically a protein, which is capable of inducing an immune response in a subject. The term also refers to proteins that are immunologically active in the sense that once administered to a subject (either directly or by administering to the subject a nucleotide sequence or vector that encodes the protein) is able to evoke an immune response of the humoral and/or cellular type directed against that protein.

As used herein, “co-administration” refers to the simultaneous administration of one or more drugs with another. In other embodiments, both drugs are administered at the same time. As described herein elsewhere, co-administration may also refer to any particular time period of administration of either drug, or both drugs. For example, as described herein, a drug may be administered hours, days, or weeks before administration of another drug and still be considered to have been co-administered. In some embodiments, co-administration may refer to any time of administration of either drug such that both drugs are present in the body of a patient at the same. In some embodiments, either drug may be administered before or after the other, so long as they are both present within the patient for a sufficient amount of time that the patient received the intended clinical or pharmacological benefits.

As used herein, the terms “effective amount” and “therapeutically effective amount” refer to an amount of an agent, vaccine, compound, drug, composition, or combination which is nontoxic and effective for producing some desired therapeutic effect upon administration to a subject or patient (e.g., a human subject or patient), such as reduce or eliminate a sign or symptom of a condition or disease. For instance, as described herein, an effective amount may be an amount necessary to treat or prevent gonococcal infection, or to measurably alter outward symptoms of gonococcal infection. In general, this amount will be sufficient to measurably inhibit bacterial replication or infectivity, or to alleviate symptoms of infection. In some examples, an “effective amount” is one that treats (including prophylaxis) one or more symptoms and/or underlying causes of any of a disorder or disease. In one example, an effective amount is a therapeutically effective amount. In one example, an effective amount is an amount that prevents one or more signs or symptoms of a particular disease or condition from developing.

As used herein, “epitope” refers to an antigenic determinant. Epitopes are particular chemical groups or peptide sequences on a molecule that are antigenic, such that they elicit a specific immune response, for example, an epitope is the region of an antigen to which B and/or T cells respond. Epitopes may be formed both from contiguous amino acids or noncontiguous amino acids juxtaposed by tertiary folding of a protein.

As used herein, “expression construct” refers to a nucleic acid construct that includes an encoded exogenous nucleic acid protein that can be transcribed and translated for functioning in the recipient to which it was administered. In some embodiments, such an expression construct may comprise DNA sequences, RNA sequences, or combinations thereof. In some embodiments, such a construct may be genetically engineered into a plasmid or vector appropriate for administration in a subject or patient, such as a particular bacterial strain or a human patient. For example, as described herein, a construct of the present disclosure may comprise a nucleic acid sequence encoding a gonococcal protein.

As used herein, “exogenous sequence” refers to a nucleic acid sequence that originates outside the host cell. An exogenous sequence may be a DNA sequence, an RNA sequence, or a combination thereof. Any type of nucleic acid available in the art may be used in accordance with the disclosure, as would be understood by one of skill in the art. Such a nucleic acid sequence can be obtained from a different species, or the same species, as that of the cell into which it is being delivered. In some embodiments, an exogenous nucleic acid sequence in accordance with the disclosure may encode a gonococcal protein as described herein, suitable for administration to a subject or patient. Such a recombinant polypeptide may be administered to a subject or patient in order to treat or prevent gonococcal and/or meningococcal infection.

As used herein, “gene delivery” refers to the introduction of an exogenous polynucleotide into a cell for gene transfer, and may encompass targeting, binding, uptake, transport, localization, replicon integration and expression.

As used herein, “gene transfer” refers to the introduction of an exogenous polynucleotide into a cell which may encompass targeting, binding, uptake, transport, localization and replicon integration, but is distinct from and does not imply subsequent expression of the gene.

As used herein, “gene expression” or “expression” refers to the process of gene transcription, translation, and post-translational modification.

As used herein, “native outer membrane vesicle” or “NOMV” refers to the outer membrane of N. meningitidis, which is composed primarily of lipooligosaccharides (LOSs), outer membrane proteins (OMPs), and phospholipids, and is normally very loosely attached to the cell wall. During stationary growth of the bacteria, vesicles or blebs of outer membrane are released into the surrounding medium. These native outer membrane vesicles (NOMV) consist of intact outer membrane, including all of the associated proteins and LOS but lacking the periplasmic and cytoplasmic components. As used herein, NOMV refers to OMV that are not treated with a detergent, i.e., “native.”

As used herein, “Neisseria gonorrhoeae” or “Ng” refers to a gonococcal bacterial strain used as described herein, which is causative for gonococcal infection.

As used herein, “Neisseria meningitidis” or “Nm” refers to a meningococcal bacterial strain used as described herein to express a gonococcal protein as described herein. Nm is causative for meningococcal infection.

By “pharmaceutically acceptable” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration. “Pharmacologically active” (or simply “active”) as in a “pharmacologically active” (or “active”) derivative or analog, refers to a derivative or analog having the same type of pharmacological activity as the parent compound and approximately equivalent in degree. The term “pharmaceutically acceptable salts” include acid addition salts which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

As used herein, “reducing” refers to a lowering or lessening, such as reducing symptoms of gonococcal infection. In some embodiments, administration of a vaccine as described herein, such as a NOMV vaccine, may result in “reduced” or lessened symptoms in the patient compared to a patient not been administered such a vaccine. “Reducing” may also refer to a reduction in disease symptoms as a result of a treatment as described herein, either alone, or co-administered with another drug.

As used herein, “subject” or “individual” or “patient” refers to any patient for whom or which therapy or treatment for gonococcal and/or meningococcal infection is desired, and generally refers to the recipient of the therapy. A “subject” or “patient” refers to any animal classified as a mammal, e.g., human and non-human mammals. Examples of non-human animals include dogs, cats, cattle, horses, sheep, pigs, goats, rabbits, etc. Unless otherwise noted, the terms “patient” or “subject” are used herein interchangeably. In some embodiments, a subject amenable for therapeutic applications of the disclosure may be a primate, e.g., human and non-human primates.

As used herein, administration of a polynucleotide or vector into a host cell or a subject refers to introduction into the cell or the subject via any routinely practiced methods. This includes “transduction,” “transfection,” “transformation,” or “transducing,” as well known in the art. These terms all refer to standard processes for the introduction of an exogenous polynucleotide, e.g., a gonococcal protein, into a host cell (e.g., N. meningitidis) leading to expression of the polynucleotide, e.g., the transgene in the cell, and includes the use of plasmids and/or recombinant viruses to introduce the exogenous polynucleotide to the host cell. Transduction, transfection, or transformation of a polynucleotide in a cell may be determined by methods well known to the art including, but not limited to, protein expression (including steady state levels), e.g., by ELISA, flow cytometry and western blot, measurement of DNA and RNA by assays, e.g., northern blots, Southern blots, reporter function (Luc) assays, and/or gel shift mobility assays. Methods used for the introduction of the exogenous polynucleotide include well-known techniques such as bacterial and/or viral infection or transfection, lipofection, transformation, and electroporation, as well as other non-viral gene delivery techniques. The introduced polynucleotide may be stably or transiently maintained in the host cell.

“Transcriptional regulatory sequences” or “TRS” of use in the present disclosure generally include at least one transcriptional promoter and may also include one or more enhancers and/or terminators of transcription. “Operably linked” refers to an arrangement of two or more components, wherein the components so described are in a relationship permitting them to function in a coordinated manner. By way of illustration, a transcriptional regulatory sequence or a promoter is operably linked to a coding sequence if the TRS or promoter promotes transcription of the coding sequence. An operably linked TRS is generally joined in cis with the coding sequence, but it is not necessarily directly adjacent to it.

The terms “treating” and “treatment” or “alleviating” or “reducing” as used herein refer to reduction or lessening in severity and/or frequency of symptoms, elimination of symptoms and/or underlying cause, and improvement or remediation of damage of, e.g., gonococcal and/or meningococcal infection. The phrase “administering to a patient” refers to the process of introducing a composition, vaccine, or dosage form into the patient via an art-recognized means of introduction. “Treating” or “alleviating” also includes the administration of compounds or agents to a subject to prevent or delay the onset of the symptoms, complications, or biochemical indicia of a disease (e.g., a gonococcal and/or a meningococcal infection), alleviating the symptoms or arresting or inhibiting further development of the disease, condition, or disorder. Subjects in need of treatment include those already suffering from the disease or disorder, as well as those being at risk of developing the disease or disorder. Treatment may be prophylactic (to prevent or delay the onset of the disease, or to prevent the manifestation of clinical or subclinical symptoms thereof) or therapeutic suppression, or alleviation of symptoms after the manifestation of the disease.

A “vector” is a nucleic acid with or without a carrier that can be introduced into a cell. Vectors capable of directing the expression of genes encoding for one or more polypeptides are referred to as “expression vectors.” Examples of vectors suitable for the present disclosure include, e.g., viral vectors, plasmid vectors, liposomes, and other gene delivery vehicles.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

Examples of embodiments of the present disclosure are provided in the following examples. The following examples are presented only by way of illustration and to assist one of ordinary skill in using the disclosure. The examples are not intended in any way to otherwise limit the scope of the disclosure.

### Example 1—Knocking Out Fhbp, siaD-galE, and lpxL1 Genes by Insertion of a Gene Coding for N. gonorrhoeae (Ng) Antigens

Transformation of N. meningitidis.

The H44/76 strain in which the fhbp, siaD-galE, and lpxL1 genes were inactivated (H44/76ΔFHbp ΔCapsule ΔlpxL1) and copies of GNA1220, MetQ, and/or MetQSM were inserted was made by homologous recombination by transformation with plasmids pBS-FHbpKO-[GNA1220, MetQ, or MetQSM]-ERM using erythromycin selection (10 μg/ml), pGEM-SiaD/GalEKO-[GNA1220, MetQ, or MetQSM]-SPC using spectinomycin selection (50 μg/ml), pUC18-lpxL1KO-[GNA1220, MetQ, or MetQSM]-KAN using kanamycin selection (50 μg/ml), and pFP12-[GNA1220, MetQ, or MetQSM]-CAT. Transformations starting from the wild-type strain were carried in the following order:

(1) The capsule genes were knocked out and the first copy of [GNA1220, MetQ, or MetQSM] was added (pGEM-SiaD/GalEKO-[GNA1220, MetQ, or MetQSM]-SPC plasmid);

(2) The lpxL1 gene was knocked out and a second copy of [GNA1220, MetQ, or MetQSM] was added (pUC18-lpxL1KO-[GNA1220, MetQ, or MetQSM]-KAN plasmid);

(3) The FHbp gene was knocked out and a third copy of [GNA1220, MetQ, or MetQSM] was added (pBS-FHbpKO-[GNA1220, MetQ, or MetQSM]-ERM plasmid).

(4) Overexpression of [GNA1220, MetQ, MetQSM, or NHBA] (pFP12-[GNA1220, MetQ, MetQSM, or NHBA]-CAT plasmid).

Of note, NHBA was expressed in a parent strain from pFP12 that had siaD-galE, LpxLa, and FHbp knocked out but not replaced with copies of NHBA.

Ten to 15 colonies of the H44/76 strain were selected from a TSB (Tryptic Soy Broth, non-animal origin) agar plate that had been grown overnight. The colonies of bacteria were mixed with 3 μg of the plasmid, plated onto a TSB agar plate, and incubated for 6 hrs at 37° C. Serial dilutions of the bacteria were re-cultured onto TSB agar plates containing antibiotic for selection. The culture plates were incubated overnight at 37° C., and the colonies were screened for GNA1220, MetQ, MetQSM, or NHBA expression and for the lack of expression of FHbp, Capsule, and lpxL1 by a flow cytometry assay using specific antibodies, and by PCR using heat killed cells. Positive individual colonies were frozen in 10% skim milk (wt/vol) and 15% glycerol, and stored at −80° C.

Primers:

Primers to go into pUC18 Lpxl1 and pBS FHbp plasmids were as follows:

MetQ WT and N238A mutant:
MetQ_neisseria forward primer:
(SEQ ID NO: 9)
5′atacaattgCCTCAGCGCATGCATC 3′
MetQ_SbfI reverse primer:
(SEQ ID NO: 10)
5′ tatCCTGCAGGTTATACGACTGCCTTATTTG 3′
GNA1220:
MetQ_neisseria forward primer:
(SEQ ID NO: 9)
5′atacaattgCCTCAGCGCATGCATC 3′
GNA1220_SbfI reverse primer:
(SEQ ID NO: 10)
5′ tatCCTGCAGGTTATACGACTGCCTTATTTG 3′
Primers to go into pGEM SiaD/GalE plasmid:
MetQ WT, N238A mutant and GNA1220:
MetQ_neisseria forward primer:
(SEQ ID NO: 9)
5′atacaattgCCTCAGCGCATGCATC 3′
MetQ_neisseria reverse primer:
(SEQ ID NO: 11)
5′tattctagaTTATACGACTGCCTTATTTGGC 3′
Primers to go into pFP12 plasmid:
MetQ WT and N238A mutant:
MetQ_neisseria forward primer:
(SEQ ID NO: 9)
5′atacaattgCCTCAGCGCATGCATC 3′
MetQ_SpeI reverse primer:
(SEQ ID NO: 12)
5′tatACTAGTTTATACGACTGCCTTATTTGGCTG 3
GNA1220:
MetQ_neisseria forward primer:
(SEQ ID NO: 9)
5′atacaattgCCTCAGCGCATGCATC 3′
GNA1220_StuI reverse primer:
(SEQ ID NO: 13)
5′tatAGGCCTTATACGACTGCCTTATTTGGC 3′
metQ pBS Downstream Forward primer:
(SEQ ID NO: 15)
5′ CCCTGTTCCAAGAGCCGAGC 3′
RBD pBS Downstream Reverse primer:
(SEQ ID NO: 16)
5′ AGCTTCTTCCAGCGCGAACG 3′, producing an 800-bp
fragment.
FHbp Upstream Forward primer (use these set of
primers for RBD as well):
(SEQ ID NO: 17)
5′GGCGAAATCGGCGTATTGGG 3′
FHbp Upstream Reverse primer:
(SEQ ID NO: 18)
5′ CTACATTACGCATTTGGAATACC 3′, producing an 800-
bp fragment.

Construction of pFP12 Shuttle Vector Containing GNA1220, MetQ, MetQSM, or NHBA with an Nm Lipoprotein Signal Sequence, or Construction of the Same with an E. coli Origin of Replication.

Characterization of mutant of Nm strain H44/76 containing 3 chromosomal copies coding for GNA1220, MetQ, MetQSM, or NHBA and a multi-copy plasmid coding for GNA1220, MetQ, MetQSM, or NHBA, each with an Nm lipoprotein signal sequence.

PCR

PCR primers were designed in order to amplify upstream and downstream the constructs inserted in Neisseria meningitidis strain H44/76 carrying the flanking region for the fhbp, siaD-galE, or lpxL1 genes, the GNA1220, MetQ, or MetQSM gene, and the antibiotic resistant cassette. PCR was performed on heat killed cells. The heat killed cells from the wild-type H44/76 were used as negative control.

Flow Cytometry

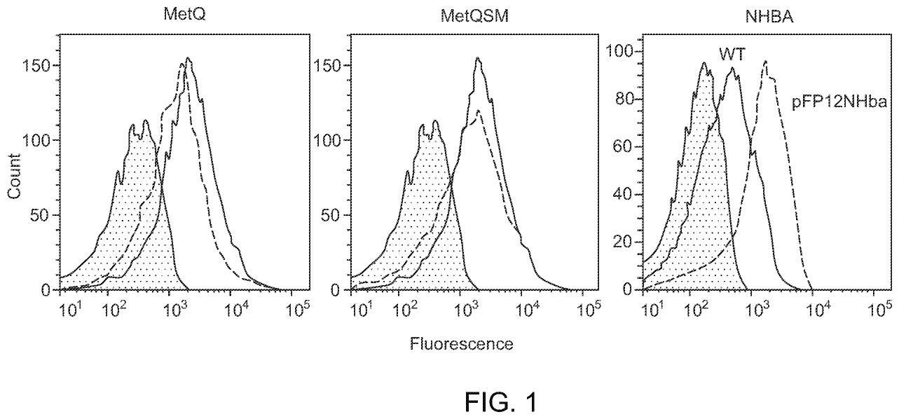

Binding of purified monoclonal and polyclonal antibodies against GNA1220, MetQ, MetQSM, or NHBA to the surface of live N. meningitidis or N. gonorrhoeae bacteria was measured by flow cytometry as described previously (Giuntini et al., Glin Vaccine Immunol 23:698-706, 2016). H44/76, engineered to expresses the target antigens, was used as the test strain. Briefly, bacteria were grown in Frantz+lactate or chemically defined medium (CDM) (Müller et al 2015, Infect Immun 83:1257-1264) containing 20 mM instead of 4 mM lactate, up to an OD_{620 nm} of 0.6-0.7. To measure anti-MetQ or anti-NHBA antibody binding, a fixed concentration of anti-MetQ or anti-NHBA antibodies or, as a negative control, 10 μg/mL of an irrelevant antibody, was incubated with 107 bacteria/mL. Bound antibody was detected using AlexaFluor 488-conjugated goat anti-mouse or rabbit IgG secondary antibody (Jackson Immuno Research Laboratories) (FIG. 1). FIG. 1 depicts enhanced binding by flow cytometry of anti-MetQ polyclonal antibodies to the lab-passaged strain of H44/76 lacking PorA in which siaD-galE, lpxL1 and fhbp loci have been disrupted with copies of genes coding for MetQ and MetQSM, respectively, and, in addition, carrying a multi-copy plasmid (example plasmid maps depicted in FIGS. 2-4) with each respective gene. Also, shown in FIG. 1, is enhanced binding of anti-NHBA polyclonal antibodies to the same parent strain in which siaD-galE, lpxL1, and fhbp loci have been disrupted, but the recombinant NHBA gene is provided only by the multi-copy pFP12-NHBA plasmid compared to expression of wild-type meningococcal NHBA naturally expressed by the strain.

Preparation and characterization of NOMV vaccine containing GNA1220, MetQ, MetQSM, or NHBA.

NOMV Preparation

Outer membrane vesicles (OMV) are prepared from a cultured strain of Neisseria meningitidis spp. genetically modified to express GNA1220, MetQ, MetQSM, or NHBA full-length proteins and derivatives. OMVs may be obtained from Neisseria meningitidis grown in broth or solid medium culture, preferably by separating the bacterial cells from the culture medium (e.g., by filtration or by a low-speed centrifugation that pellets the cells, or the like), lysing the cells (e.g., by addition of detergent, osmotic shock, sonication, cavitation, homogenization, or the like) and separating an outer membrane fraction from cytoplasmic molecules (e.g., by filtration; or by differential precipitation or aggregation of outer membranes and/or outer membrane vesicles, or by affinity separation methods using ligands that specifically recognize outer membrane molecules; or by a high-speed centrifugation that pellets outer membranes and/or outer membrane vesicles, or the like); outer membrane fractions may be used to produce OMVs.

OMVs were obtained from Neisseria meningitidis grown in Frantz+lactate or chemically defined medium (CDM) (Müller et al 2015, Infect Immun 83:1257-1264) containing 20 mM instead of 4 mM lactate, inoculated with bacteria to an OD_{620 nm} of 0.15-0.2 from overnight colonies of bacteria on TSB (Tryptic Soy Broth, non-animal origin) agar plates. The culture was incubated at 37° C. in 5% CO₂, and the volume of medium was sequentially increased, starting from individual colonies inoculated into 24 mL of medium at OD_{620 nm}=˜0.15 to 1 L by transferring the culture to the next larger volume as the OD_{620 nm} reached 0.6-0.7 (i.e., 24 mL to 90 mL to 300 mL to 1 L). When the final volume was reached, the culture was left to grow for an additional 15 hours in a shake flask with vented enclosure. The bacteria were then centrifuged (10,000×g, 20 minutes), the supernatant filtered through a glass fiber filter to remove debris, then sterile-filtered (0.22 μm filter), and concentrated by ultrafiltration (100 k or 30 k cutoff filter, Amicon) and benzonase added (1000 U/L). Benzonase treatment was continued for at least 1 hr at ambient temperature. The concentrated filtrate was centrifuged (202,601×g, 1.5 hrs, 4° C.) to collect the NOMV. The NOMV were suspended in 10 mM Tris·HCl, pH 7.4, 3% (w/v) sucrose, centrifuged again as described in the previous step, and finally suspended in the Tris/sucrose solution to a concentration between 1 to 3 mg/ml protein as determined by DC Protein Assay (Bio-Rad). The NOMV preparation was stored frozen at −70° C. until used.

ELISA Assay

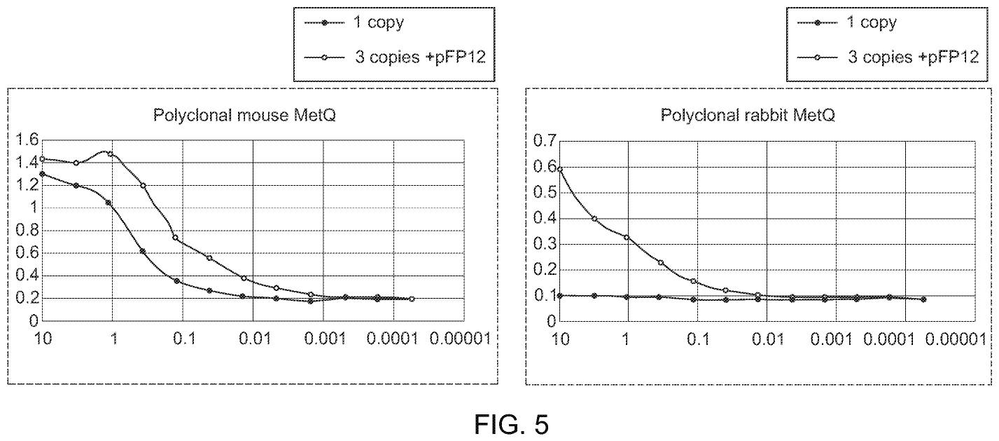

To determine expression of MetQ or MetQSM in NOMV vaccine (NOMV-designated protein), 96-well plates (Nunc) were coated overnight at 4° C. with a titration of purified NOMV-MetQ or NOMV-MetQSM. Plates were blocked with 1% BSA+0.05% Tween 20 in PBS. A fixed concentration (1 μg/ml) of MetQ polyclonal antibodies were diluted in PBS+0.1% Tween 20 and added to plates for 2 hours. Plates were stained with alkaline phosphatase-conjugated goat anti-mouse IgG (Jackson Immuno Research Laboratories) (1:2,000) or goat anti-rabbit IgG (Jackson Immuno Research Laboratories) (1:2,000) for 1 hour and developed using p-nitrophenyl phosphate (Thermo Fisher Scientific). Results for MetQ and MetQSM are depicted in FIG. 5.

### Example 2—Immunization

The NOMV-GNA1220, NOMV-MetQ, NOMV-MetQSM, or NOMV-NHBA preparation or recombinant protein was diluted in 10 mM Tris·HCl, pH 7.4, 3% (w/v) sucrose and adsorbed with an equal volume of aluminum hydroxide adjuvant (2% ALHYDROGEL, Invivogen). Vaccines were prepared the evening before the immunization and incubated overnight at 4° C. Groups of 4-6-week-old female CD1 mice (Charles River Breeding Laboratories) (N=10 per group) were immunized intraperitoneally (IP). Each mouse received a dose containing 10-2.5 μg of total protein of NOMV or 10 ug of recombinant GNA1220, MetQ, MetQSM, or NHBA pre-mixed with 600 μg of adjuvant. A total of three injections were given, each separated by 3-week intervals. Two weeks after the third dose, mice were bled by cardiac puncture and sacrificed. The sera were separated and stored frozen at −80° C.

### Example 3—Intranasal Immunization with NOMV-GNA1220 or NOMV-MetQ

To determine whether protective mucosal antibody response can be produced by intranasal vaccination, CD1 mice will be vaccinated with 50 μg of NOMV-GNA1220, NOMV-MetQ, or NOMV-MetQSM vaccine intranasally. With the mice under isoflurane anesthesia, 10 μl of vaccine preparation will be applied to each nare, which is inhaled. Mice will be immunized 2 to 3 times separated by 3-4 weeks. One intraperitoneal injection for 5 μg of vaccine will also be combined with 1 to 2 intranasal treatments.

### Example 4—Characterization of Antibody Responses to the NOMV Vaccine Containing the Protein in CD1 Mice

For binding activity of polyclonal antibodies raised in mice immunized with recombinant MetQ (rMetQ), NOMV-MetQ, NOMV-MetQSM vaccine, 96-well plates (Nunc) were coated overnight at 4° C. with 2 μg/m1rMetQ protein. Plates were blocked with 1% BSA+0.05% Tween 20 in PBS. Sera from immunized mice were diluted in PBS+0.1% Tween 20 and added to plates for 2 hours. Plates were stained with alkaline phosphatase-conjugated goat anti-mouse IgG (Jackson Immuno Research Laboratories) (1:3,000) secondary antibody for 1 hour and developed using p-nitrophenyl phosphate (Thermo Fisher Scientific). Absorbance at an OD of 405 nm was measured on an Emax precision plate reader (Molecular Devices).

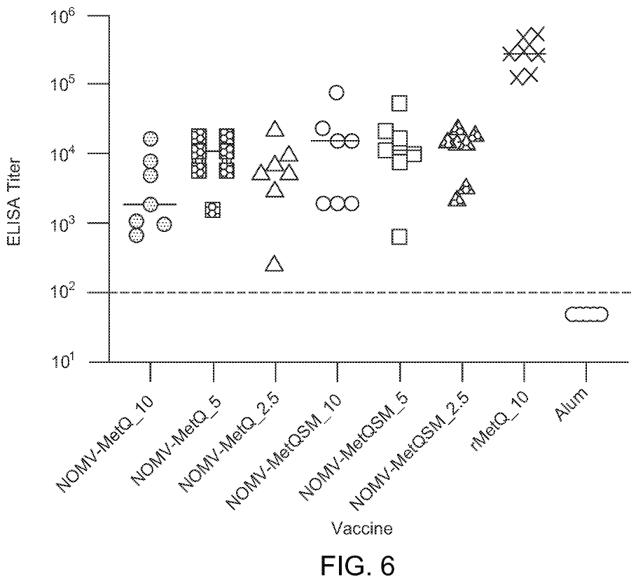

FIG. 6 shows that the IgG titers were similar for mice immunized with 2.5-10 μg of NOMV vaccines except for mice given 10 μg doses of NOMV-MetQ, where the mean titer was significantly lower when compared to all other groups. The mean IgG titer for mice immunized with rMetQ was significantly higher (p<0.01) than all other groups.

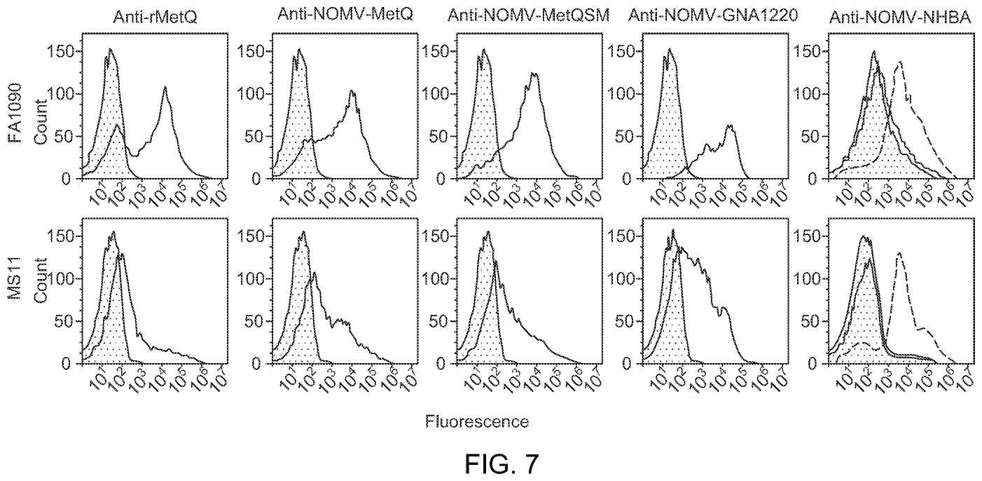

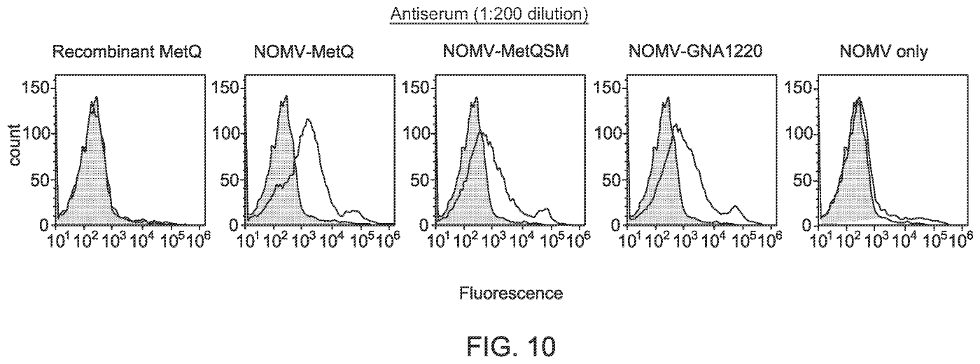

Flow cytometry was used to compare binding of polyclonal antibodies from mice immunized with rMetQ, rNHBA, NOMV-MetQ, NOMV-MetQSM, NOMV-GNA1220, and NOMV-NHBA to live bacteria. The binding assay to gonococcal strains FA1090 and MS11 and meningococcal serogroup B strain MD1224 was performed as described above. FIG. 7 shows that all of the vaccines elicited antibodies that bind to both gonococcal strains tested. FIG. 10 shows that only antibodies produced by immunization with NOMV-MetQ, NOMV-MetQSM and NOMV-GNA1220 bind to Nm strain MD1224 at a 1:200 dilution of antiserum.

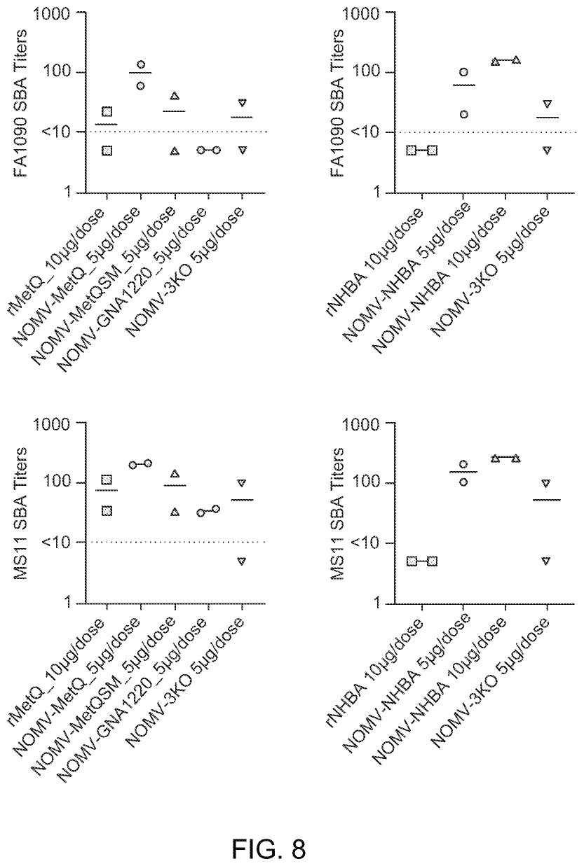

Serum bactericidal activity (SBA) of polyclonal antibodies from mice immunized with rMetQ, rNHBA, NOMV-MetQ, NOMV-MetQSM, NOMV-GNA1220, and NOMV-NHBA against Ng strains FA1090 and MS11. Bacteria were grown overnight on chocolate agar supplemented with IsoVitaleX™ (Fischer Scientific) or equivalent plate at 37° C. with 5% CO₂ and passaged the next day to a pre-warmed chocolate agar IsoVitaleX™ or equivalent plate. The plate was incubated for 5 hours at 37° C. with 5% CO₂. The bacteria were suspended in Hanks Balanced Salt Solution (HBSS) containing 0.15 mM CaCl₂ and 1 mM MgCl₂ (HBSS++) with 0.1% BSA to a OD_{620 nm} of 0.6. A 1:12500 final dilution in HBSS++ with 0.1% BSA (to obtain ˜5×10⁴ cfu/ml) was achieved on the bactericidal 96 well-plate. Sera from immunized mice were depleted from IgM before the assay using Goat anti-mouse IgM (μ-chain specific)-agarose antibody (Millipore) and serially diluted in HBSS++. Twenty percent (volume/volume) of IgG and IgM depleted human serum was used as complement source and added to each well of the 96 well-plate. The plate was incubated for 30 min at 37° C. with 5% CO₂ and serial dilutions were plated to determine colony-forming units (cfu). Though immunization with recombinant proteins produced equal or higher antibody titers with similar binding to both gonococcal strains, NOMV-MetQ and NOMV-NHBA had greater SBA activity against gonococcal strains FA1090 and MS11 than the antibodies elicited by the corresponding recombinant proteins as depicted in FIG. 8.

The effect of polyclonal antibodies from mice immunized with rMetQ, rNHBA, NOMV-MetQ, NOMV-MetQSM, NOMV-GNA1220 and NOMV-NHBA on colonization was tested with gonococcal strains FA1090 and MS11. ME180 cells (ATCC HTB33), an epithelial cell-like cell line derived from a human cervical carcinoma, was maintained in McCoy's 5A medium supplemented with 10% (volume/volume) of fetal calf serum and penicillin (100 U/ml)-streptomycin (1 mg/ml). For adherence assays, the cells were seeded into 96-well plates at 2.5×10⁵ cells/well and incubated in 5% CO₂ at 37° C. for 24 hours. Nonconfluent monolayers (70-80% confluence) were overlaid with 100 μl of bacteria (107 bacteria/m1), incubated for 1 h in 5% CO₂ at 37° C., and washed three times for 5 min each time in phosphate-buffered saline (PBS, pH 7.4). Acutase (100 μl/well) was added for 15 min at 37° C. and serial dilutions were plated to determine colony-forming units (cfu). Gonococci colonize different biological niches that pose different nutritional stresses on the bacteria. The variable nutritional circumstances result in expression of different proteins on the surface of the bacteria. The polyclonal antibodies were tested for the effect on FA1090 and MS11 colonization in two different conditions. As depicted in FIG. 10, colonization was most strongly inhibited by anti-NOMV-GNA1220 and anti-NOMV-NHBA when bacteria were grown on chocolate agar plates, while anti-NOMV-MetQSM and anti-NOMV-NHBA had the greatest effect on bacteria grown in liquid culture in CDM containing 10% (volume/volume) IgG/IgM-depleted human serum.

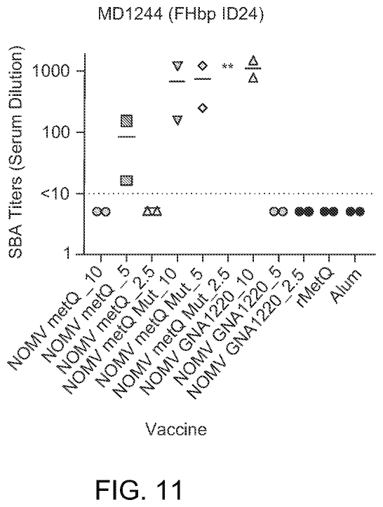

Serum bactericidal activity (SBA) of polyclonal antibodies from mice immunized with 2 doses of 2.5 μg, 5 μg, or 10 μg of NOMV-MetQ, NOMV-MetQSM, NOMV-GNA1220, adjuvant alone or 10 μg of recombinant MetQ (rMetQ) was determined as described in Beernink et al. J Infect Disease 219:1131, 2019. As depicted in FIG. 11, mice with NOMV-MetQ but particularly NOMV-MetQSM were effective in mediating SBA with human complement, which is a correlate of protection in humans.

Overall, the data depicted provide evidence that antibodies produced by immunization with conserved gonococcal antigens in meningococcal NOMV have greater binding to gonococcal and meningococcal strains, SBA and inhibition of colonization functional activity than antibodies produced by immunization with the corresponding recombinant proteins.

### Example 5—Conditionally Reprogrammed Human Epithelial Cell Culture (CRC) Models for Evaluating the Protection by Vaccine-Elicited Antibodies on the Early Stages of Nm and Ng Infection

The ability to produce human primary epithelial cell cultures by reprogramming that have characteristics of tissues is relatively new and the use of them to evaluate the effect of vaccine elicited antibodies on Nm colonization, pathogenesis, and protection against both is novel. A particular interest is in protection by vaccine-elicited antibodies at the earliest stages of infection since this has been historically less well studied yet critical for the control of disease in large populations. Immortalized human cell lines (e.g., 16HBE14o− and ME180) are available but do not replicate the variety of cell types and characteristics of CRC cells. Human tissue explants are also important but are meagerly available, making it difficult to perform the number of experiments needed to compare the effects of antibodies elicited by the relatively large number of vaccines proposed to be tested. A primary nasal epithelial (pNE) model of meningococcal colonization was established and similar methods will be used (Suprynowicz et al., Proc Natl Acad Sci USA 2012; 109(49):20035-40) to establish a primary cervical model of Ng colonization. Together, the primary human cell culture models provide an important and innovative approach for evaluating the potential of vaccine-elicited antibodies to affect the course of Nm and Ng colonization and invasion. ME180 human cervical cells were used for the adhesion studies described herein, and adhesion to CRC cells will also be studied.

### Example 6—Human CEACAM1/FH Transgenic (Tg) Mouse Model for Evaluating the Protection by Vaccine-Elicited Antibodies on the Early Stages of Nm and Ng Infection

A unique human transgenic (Tg) mouse model was produced with a functional complement system expressing human genes that facilitate colonization (CEACAM1) and immune protection (FH) that is colonized by Nm through intranasal infection and rapidly develops meningitis-like symptoms with migration of the bacteria from the nasopharynx to the meninges surrounding the brain. In addition, technical improvements to the Tg mouse model have been made that distinguish adherent from non-adherent bacteria isolated from the nasopharynx. The human CEACAM1/FH Tg mice may also be useful for measuring protection by vaccine-elicited antibodies against Ng colonization in the estradiol-treated female mouse model (Jerse et al., Front Microbiol 2011; 2:107) since CEACAM1 is expressed in these mice in columnar epithelial cells that line the surface of the uterus and endocervix where Opa binding to CEACAM1 enhances Ng association and penetration into these tissues (Islam et al., Infect Immun 2018; 86(8)). In addition, human FH binding by NspA, PorB2, and LOS derivatives provides immune shielding and, possibly, additional mechanisms for epithelial cell adhesion.

### Example 7—Electroporation to Increase the Amount of DNA in NOMV

Electroporation to increase the amount of plasmid in NOMV was evaluated. In order to increase electroporation efficiency, different ratios of NOMV:plasmid DNA, different electroporation voltages, and number of pulses was tested. As a result of electroporation, the content of dsDNA was increased in the NOMV up to 5-fold, compared to what was present in cells without electroporation.

NOMV and plasmid DNA were electroporated at a 2:1 ratio or at a 1:1 ratio in 300 mM Sucrose buffer. See Tables 1 and 2 below for details. After electroporation, samples were incubated with Benzonase overnight in order to eliminate any residual/external DNA. After Benzonase treatment, NOMV were lysed with a 1% SDS solution and dsDNA was measured using Qubit™ 1×dsDNA HS Assay Kit.

TABLE 1
Parameters for Electroporation.

| 2:1 Ratio NOMV:pFP12 | dsDNA ng/ug of NOMV | Fold increase |
|---|---|---|
| No electroporation | 0.94 | |
| 800 V 2X pulse | 1.9 | 2 |
| 900 V 2X pulse | 2.9 | 3 |
| 1000 V 2X pulse | 3.12 | 3.3 |

TABLE 2
Parameters for 900 V 2X pulse Electroporation.

| 2X Pulse | dsDNA ng/ug of NOMV | Fold increase |
|---|---|---|
| No electroporation | 0.76 | |
| 2:1 ratio NOMV:0FP12 | 2.28 | 3 |
| 1:1 ratio NOMV:pFP12 | 3.8 | 5 |

What is claimed is:

1. A pharmaceutical vaccine composition comprising a plurality of bacterial native outer-membrane vesicles (NOMVs) comprising at least one recombinant protein from Neisseria gonorrhoeae, wherein the gonococcal recombinant protein is a lipoprotein or is modified to be a lipoprotein.

2. The pharmaceutical vaccine composition of claim 1, wherein the gonococcal recombinant protein is modified by eliminating portions of the protein that are not surface exposed and adding a lipoprotein signal sequence to the remaining C-terminal portion, wherein the gonococcal recombinant protein is displayed on the surface of the bacteria and NOMV are produced by the bacteria as a lipoprotein.

3. The pharmaceutical vaccine composition of claim 1 or 2, wherein the at least one gonococcal recombinant protein is GNA1220, MetQ, MetQSM, or NHBA, or derivatives or fragments thereof, or combinations thereof.

4. The pharmaceutical vaccine composition of claim 1, wherein the NOMVs are derived from Neisseria meningitidis.

5. The pharmaceutical vaccine composition of claim 4, wherein the meningococcal strain is H44/76.

6. A strain of Neisseria meningitidis comprising at least one gene encoding at least one recombinant protein from Neisseria gonorrhoeae, wherein the at least one gonococcal recombinant protein is a lipoprotein or is modified to be a lipoprotein.

7. The meningococcal strain of claim 6, wherein the at least one gonococcal recombinant protein is GNA1220, MetQ, MetQSM, or NHBA, or derivatives or fragments thereof, or combinations thereof.

8. The meningococcal strain of claim 7, wherein the at least one gonococcal recombinant protein is expressed from a transgene in a plasmid.

9. The meningococcal strain of claim 7, wherein the at least one gonococcal recombinant protein is expressed from a transgene inserted in the bacterial genome.

10. The meningococcal strain of any of claims 6-9, wherein the meningococcal strain is H44/76.

11. The meningococcal strain of claim 10, wherein the meningococcal strain H44/76 does not express porin PorA.

12. The meningococcal strain of any of claims 6-11, wherein expression of the transgene encoding the at least one gonococcal recombinant protein is driven by a strong promoter sequence that produces high rates of gene transcription in Neisseria meningitidis.

13. The meningococcal strain of claim 12, wherein the strong promoter comprises a PorA promoter or a derivative thereof.

14. The meningococcal strain of any of claim 13, wherein the promoter comprises a sequence set forth in FIGS. 2-4.

15. The meningococcal strain of claims 6-14, wherein the transgene encoding the at least one gonococcal recombinant protein is inserted into the lpxL1 locus of the bacterial genome, wherein the insertion disrupts expression of the acyltransferase gene, and wherein the disruption causes the bacteria to produce a lipooligosaccharide that is penta-acylated and not hexa-acylated.

16. The meningococcal strain of any of claims 6-14, wherein the transgene encoding the at least one gonococcal recombinant protein is inserted into the siaD-galE locus of the bacterial genome, and wherein the insertion disrupts expression of the capsular polysaccharide and sialylation of the lipooligosaccharide host antigens.

17. The meningococcal strain of any of claims 6-14, wherein the transgene encoding the at least one gonococcal recombinant protein is inserted into the siaA locus.

18. The meningococcal strain of any of claims 6-17, wherein the transgene encoding the at least one gonococcal recombinant protein is inserted into the fhbp locus (Factor H binding protein).

19. The meningococcal strain of any of claims 6-17, wherein the transgene encoding the at least one gonococcal recombinant protein is inserted into the porA locus.
